SEQUENCE LISTING (28 sequences)

SEQ ID NO: 1 - RNA, 107 nt, Artificial Sequence
  sgRNA-198
  misc_feature: 2′-O-methyl modification and phosphorothioate inter-nucleotide linkage
  misc_feature (2)..(2): 2′-O-methyl modification and phosphorothioate inter-nucleotide linkage
  misc_feature (4)..(4): 2′-O-methyl modification and phosphorothioate inter-nucleotide linkage
  misc_feature (6)..(6): 2′-O-methyl modification and phosphorothioate inter-nucleotide linkage
  misc_feature (102)..(102): 2′-O-methyl modification and phosphorothioate inter-nucleotide linkage
  misc_feature (104)..(104): 2′-O-methyl modification and phosphorothioate inter-nucleotide linkage
  misc_feature (106)..(106): 2′-O-methyl modification and phosphorothioate inter-nucleotide linkage
gngnunggggaaggggcccccaagguuuuagagcuagaaauagcaaguuaaaauaaggcuaguccguuaucaacuugaaaaaguggcaccgagucggugcunununu

SEQ ID NO: 2 - RNA, 107 nt, Artificial Sequence
  sgRNA-175
  misc_feature: 2′-O-methyl modification and phosphorothioate inter-nucleotide linkage
  misc_feature (2)..(2): 2′-O-methyl modification and phosphorothioate inter-nucleotide linkage
  misc_feature (4)..(4): 2′-O-methyl modification and phosphorothioate inter-nucleotide linkage
  misc_feature (6)..(6): 2′-O-methyl modification and phosphorothioate inter-nucleotide linkage
  misc_feature (102)..(102): 2′-O-methyl modification and phosphorothioate inter-nucleotide linkage
  misc_feature (104)..(104): 2′-O-methyl modification and phosphorothioate inter-nucleotide linkage
  misc_feature (106)..(106): 2′-O-methyl modification and phosphorothioate inter-nucleotide linkage
gnangnauauuugcauugagauagguuuuagagcuagaaauagcaaguuaaaauaaggcuaguccguuaucaacuugaaaaaguggcaccgagucggugcunununu

SEQ ID NO: 3 - RNA, 107 nt, Artificial Sequence
  sgRNA-113
  misc_feature (2)..(2): 2′-O-methyl modification and phosphorothioate inter-nucleotide linkage
  misc_feature (4)..(4): 2′-O-methyl modification and phosphorothioate inter-nucleotide linkage
  misc_feature (6)..(6): 2′-O-methyl modification and phosphorothioate inter-nucleotide linkage
  misc_feature (102)..(102): 2′-O-methyl modification and phosphorothioate inter-nucleotide linkage
  misc_feature (104)..(104): 2′-O-methyl modification and phosphorothioate inter-nucleotide linkage
  misc_feature (106)..(106): 2′-O-methyl modification and phosphorothioate inter-nucleotide linkage
gncnunugaccaauagccuugacaguuuuagagcuagaaauagcaaguuaaaauaaggcuaguccguuaucaacuugaaaaaguggcaccgagucggugcunununu

SEQ ID NO: 4 - DNA, 5412 bp, Artificial Sequence
  ABE-max NG SpCas9 plasmid ORF
atgaaacggacagccgacggaagcgagttcgagtcaccaaagaagaagcggaaagtctctgaagtcgagtttagccacgagtattggatgaggcacgcactgaccctggcaaagcgagcatgggatgaaagagaagtccccgtgggcgccgtgctggtgcacaacaatagagtgatcggagagggatggaacaggccaatcggccgccacgaccctaccgcacacgcagagatcatggcactgaggcagggaggcctggtcatgcagaattaccgcctgatcgatgccaccctgtatgtgacactggagccatgcgtgatgtgcgcaggagcaatgatccacagcaggatcggaagagtggtgttcggagcacgggacgccaagaccggcgcagcaggctccctgatggatgtgctgcaccaccccggcatgaaccaccgggtggagatcacagagggaatcctggcagacgagtgcgccgccctgctgagcgatttctttagaatgcggagacaggagatcaaggcccagaagaaggcacagagctccaccgactctggaggatctagcggaggatcctctggaagcgagacaccaggcacaagcgagtccgccacaccagagagctccggcggctcctccggaggatcctctgaggtggagttttcccacgagtactggatgagacatgccctgaccctggccaagagggcacgcgatgagagggaggtgcctgtgggagccgtgctggtgctgaacaatagagtgatcggcgagggctggaacagagccatcggcctgcacgacccaacagcccatgccgaaattatggccctgagacagggcggcctggtcatgcagaactacagactgattgacgccaccctgtacgtgacattcgagccttgcgtgatgtgcgccggcgccatgatccactctaggatcggccgcgtggtgtttggcgtgaggaacgcaaaaaccggcgccgcaggctccctgatggacgtgctgcactaccccggcatgaatcaccgcgtcgaaattaccgagggaatcctggcagatgaatgtgccgccctgctgtgctatttctttcggatgcctagacaggtgttcaatgctcagaagaaggcccagagctccaccgactccggaggatctagcggaggctcctctggctctgagacacctggcacaagcgagagcgcaacacctgaaagcagcgggggcagcagcggggggtcagacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatccggcccaagaggaacagcgataagctgatcgccagaaagaaggactgggaccctaagaagtacggcggcttcgtgagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgcccggttcctgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctcgggccttcaagtactttgacaccaccatcgaccggaaggtgtaccggagcaccaaagaggtgctggacgccaccctgatccaccagagcatcaccggcctgtacgagacacggatcgacctgtctcagctgggaggtgactctggcggctcaaaaagaaccgccgacggcagcgaattcgagcccaagaagaagaggaaagtctaa

SEQ ID NO: 5 - DNA, 25 bp, Artificial Sequence
  hHBG.F primer
tgactgaatcggaacaaggcaaagg

SEQ ID NO: 6 - DNA, 23 bp, Artificial Sequence
  hHBG.R primer
attcttcatccctagccagccgc

SEQ ID NO: 7 - DNA, 20 bp, Artificial Sequence
  sgRNA-1
acccggaactccctcaagca

SEQ ID NO: 8 - DNA, 20 bp, Artificial Sequence
  sgRNA-2
atttccattagattcattag

SEQ ID NO: 9 - DNA, 26 bp, Artificial Sequence
  4.9kb Forward primer
acggataagtagatattgaggtaagc

SEQ ID NO: 10 - DNA, 21 bp, Artificial Sequence
  4.9kb Reverse primer
gtctctttcagttagcagtgg

SEQ ID NO: 11 - DNA, 27 bp, Artificial Sequence
  Taqman probe
actgcgctgaaactgtggtctttatga

SEQ ID NO: 12 - DNA, 20 bp, Artificial Sequence
  Proximal promoter
ctatctcaatgcaaatatct

SEQ ID NO: 13 - DNA, 20 bp, Artificial Sequence
  g22#1 (A)
agatatttgcattgagatag

SEQ ID NO: 14 - DNA, 20 bp, Artificial Sequence
  g22#2 (C)
gatatttgcattgagatagt

SEQ ID NO: 15 - DNA, 20 bp, Artificial Sequence
  g22#3 (D)
cagatatttgcattgagata

SEQ ID NO: 16 - DNA, 20 bp, Artificial Sequence
  g22#4 (E)
acagatatttgcattgagat

SEQ ID NO: 17 - DNA, 20 bp, Artificial Sequence
  G22#5 (F)
gacagatatttgcattgaga

SEQ ID NO: 18 - DNA, 23 bp, Artificial Sequence
  new KLF1 binding motif
cctcttgggggccccttccccac

SEQ ID NO: 19 - DNA, 23 bp, Artificial Sequence
  complement to new KLF1 binding motif
ggagaacccccggggaaggggtg

SEQ ID NO: 20 - DNA, 22 bp, Artificial Sequence
  new TAL1 binding motif
cactatctcaatgcaaatatct

SEQ ID NO: 21 - DNA, 22 bp, Artificial Sequence
  complement to new TAL1 binding motif
gtgatagagttacgtttataga

SEQ ID NO: 22 - DNA, 23 bp, Artificial Sequence
  new GATA1 binding motif
cttgaccaatagccttgacaagg

SEQ ID NO: 23 - DNA, 23 bp, Artificial Sequence
  complement to new GATA1 binding motif
gaactggttatcggaactgttcc

SEQ ID NO: 24 - DNA, 45 bp, Artificial Sequence
  GATA proximal to TAL1
cacactatctcaatgcaaatatctgtctgaaacggtccctggcta

SEQ ID NO: 25 - DNA, 45 bp, Artificial Sequence
  complement to GATA proximal to TAL1
gtgtgatagagttacgtttatacacagactttgccagggaccgat

SEQ ID NO: 26 - DNA, 35 bp, Artificial Sequence
  GATA motif proximal to TAL1 binding site
ccccttccccacactatctcaatgcaaacatctgt

SEQ ID NO: 27 - DNA, 35 bp, Artificial Sequence
  complement to GATA motif proximal to TAL1 binding site
ggggaaggggtgtgatagagttacgtttgtagaca

SEQ ID NO: 28 - DNA, 35 bp, Artificial Sequence
  HUDEP2 -175/-187&-189
ggggaaggggtgtggtggagttacgtttgtagaca

(19) United States
(12) Patent Application Publication
(10) Pub. No.: US 2024/0360414 A1
(43) Pub. Date: Oct. 31, 2024

(54) Systems and Methods for Base Editing Of HBG1/2 Gene Promoter and Fetal Hemoglobin Induction

(71) Applicant: St. Jude Children's Research Hospital, Inc., Memphis, TN (US)

(72) Inventors: Thiyagaraj MAYURANATHAN, Memphis, TN (US); Jonathan YEN, Memphis, TN (US); Mitchell J. WEISS, Memphis, TN (US)

(21) Appl. No.: 18/272,536

(22) PCT Filed: Jan. 14, 2022
(86) PCT No.: PCT/US22/12497

§ 371 Date: Jul. 14, 2023

Related U.S. Application Data
(60) Provisional application No. 63/137,919, filed on Jan. 15, 2021.

(51) Int. Cl. C12N 5/0789

(52) U.S. Cl. CPC C12N 5/0647; C12N 2510/00

(57) ABSTRACT

Systems, methods, and compositions for the introduction of mutations to the proximal promoter region of the HBG1 and HBG2 genes are provided. Also included are cells and compositions of cells bearing one or more synthetic alleles formed by the disclosed systems, methods, and compositions. This disclosure also provides a method of treating a subject by introducing the disclosed synthetic allele(s) into a cell of a subject.

### CROSS-REFERENCE TO RELATED APPLICATIONS

This application is a U.S. National Stage Entry of International Patent Application No. PCT/US2022/012497, filed Jan. 14, 2022, which claims the benefit of priority to U.S. Prov. Pat. App. No. 63/137,919 filed Jan. 15, 2021, which are incorporated by reference herein in their entirety.

### GOVERNMENT SUPPORT CLAUSE

This invention was made with government support under grant no. HL053749 awarded by the National Institutes of Health. The government has certain rights in the invention.

### SEQUENCE LISTING

The instant application contains a Sequence Listing which has been submitted electronically in ASCII form and is hereby incorporated by reference in its entirety. Said ASCII copy, created on Jan. 14, 2022, is named Sequence-Listing_SJ-20-0020_41901-7.TXT and is 14,644 bytes in size.

### FIELD OF THE DISCLOSURE

The present disclosure relates to the field of genome editing and modification of gene expression thereby, and more particularly to alteration of sequences and/or expression of genes in the beta-globin gene cluster.

### BACKGROUND OF THE DISCLOSURE

Beta-hemoglobinopathies (beta-thalassemia and sickle-cell disease (SCD)) are the two most common human genetic disorders and they represent a major health problem affecting millions world-wide. These conditions are caused by the genetic lesions that affect beta-globin gene. Beta-thalassemia is a quantitative disorder where the reduced production of beta chains cause alpha-globin precipitation in red blood cells (RBCs) causing in effective erythropoiesis. Whereas, SCD is a qualitative disorder, where the single amino acid change (beta⁶Glu→Val) leads to hemoglobin polymerization resulting in RBCs sickling causing vaso-occlusion and multiorgan damage with substantial morbidity and early mortality.

Allogeneic hematopoietic stem cell transplantation (HSCT), ideally from an HLA-matched sibling donor, provides an approved cure for beta-hemoglobinopathies. However, most patients do not have optimal donors and the procedure carries numerous risks including graft rejection and graft versus host disease (GVHD) requiring prolonged immunosuppression. Allogeneic HSCT from a haploidentical or matched unrelated donor is possible, but with greater risks. Gene therapy using autologous HSCs can potentially overcome these limitations. Delivery of modified beta-globin gene via lentiviral vector demonstrated a clinical benefit and promising strategy for patients with beta-thalassemia and SCD. Thus, while this approach shows great promise. not all patients are cured. The best curative approach(es) for beta-hemoglobinopathies are not known and multiple methods must be explored simultaneously.

Co-inheritance of mutations in the gamma-globin gene (HBG2 and HBG1) promoter causes sustained fetal hemoglobin (HbF) production in adults. a condition termed as hereditary persistence of fetal hemoglobin (HPFH) ameliorates the clinically severity of beta-hemoglobinopathies. Numerous HPFH mutations have been identified at positions −200, −175 and −115 base pairs upstream of the gamma-globin gene transcription start sites. These mutations either create de-novo binding sites for transcriptional activators (FIG. 1a) or disrupt the binding of transcriptional repressors (BCL11A and ZBTB7A) to gamma-globin promoter to active HbF. All of these regions represent target sites for induction of HbF by genome editing.

Genome editing via CRISPR/Cas9 or related nucleases represents a promising approach to cure b-hemoglobinopathies. Recently. the inventors and others have demonstrated that CRIPSR/Cas9 mediated disruption of BCL11A and ZBTB7A binding sites in gamma-globin promoter has raised the HbF in vitro and in vivo. Additionally. several groups showed that genome editing-mediated disruption of an erythroid-specific BCL11A enhancer in hematopoietic stem and progenitor cells (HSPCs) induces RBC HbF to potential therapeutic levels in-vitro and in-vivo. As HBG1 and HBG2 are nearly identical and arranged in tandem, targeting the BCL11A or ZBTB7A binding site resulted in simultaneous DSBs in both gene promoters. with 9 to 20% of cells exhibiting a deletion of the intervening 4.9 kb. In addition, simultaneous DSBs can result in loss or inversion of the intervening genetic material and/or chromosomal rearrangements. These products of Cas9 nuclease cleavage can evoke cytotoxic DNA damage responses.

Base editors are precise CRISPR/Cas9-derived tools that can install desired transition mutations without generating double stranded DNA breaks (DSBs). Adenosine base editors (ABEs) cause targeted deamination of adenosine to form inosine, which pairs with C during DNA replication. repair and transcription. leading to an A-to-G edit. ABE7.10 is a particularly attractive ABE variant due to its high on-target activity and low propensity to generate spurious off-target mutations. ABEs can install several naturally occurring HPFH mutations in HBG1 and HBG2 promoters.

A recent study demonstrated that a cytosine base editor use of CBEs serves as the erythroid-specific BCL11A enhancer in SCD and beta-thalassemia patient CD34+ HSPCs, resulting in alleviation of gamma-globin repression that is otherwise caused by BCL11a. A proximal approach was employed involving the creation of HPFH point mutations directly in the gamma-globin gene promoter that induce HbF in adult erythroid cells.

### BRIEF DESCRIPTION OF THE DISCLOSURE

Embodiments of this disclosure utilize ABEs (including, without limitation. ABE7.10) to mimic naturally occurring HPFH mutation in the gamma-globin gene promoter that induced HbF in adult erythroid cells. HPFH mutation, −175 T→C induced robust HbF in erythroid progeny derived from healthy and SCD patients. Recreation of this mutation in repopulating HSPCs will be promising therapeutic strategy to treat b-hemoglobinopathies.

In one aspect. this disclosure relates to a cell derived from a subject affected by a disease of red blood cell structure or function (e.g., a hemoglobinopathy) which comprises at least one synthetic allele of an HBG1 or HBG2 gene, said synthetic allele comprising a T→C mutation at a position at one or more of the following: −175 base pairs of the promoter region of the HBG1 or HBG2 gene: HBG2: GRCh/hg19; chr11:5.276.186; and HBG1: GRCh/hg19; chr11:5.271.262.

Cells according to this aspect may have a T→C mutation at position 175 bp (−175) upstream from 5′ UTR region or 228 bp (−228) upstream from transcription start site of HBG1/2 gene. relative to the proximal promoter sequence 5′-CTATCTCAATGCAAATATCT-3′ (SEQ ID NO: 12), e.g., at position 12 thereof. A cell according to this aspect of the disclosure may be characterized in that a differentiated cell in the erythrocyte lineage derived therefrom expresses a quantity of g-globin protein greater than the quantity of g-globin protein expressed by a cell in the erythrocyte lineage derived from the same subject which lacks the at least one synthetic allele. Cells according to this aspect may have at least one modified allele.

In another aspect. this disclosure relates to a composition of cells in which at least 20%, 25%, 30%, 35%, 40%, 45%, 50%, 55%, 60%, 65%, 70%, 75%, 80%, 85%, 90%, 95%, or more alleles of HBG1 and HBG2 genes within said composition of cells comprise a T→C mutation at −175 base pairs of a 5′ UTR of the HBG1 or HBG2 gene (i.e., 228 bp (−228) upstream from transcription start site). In some cases, compositions of cells according to this aspect may be differentiated into an erythrocyte lineage, in which case at least 20%, 25%, 30%, 35%, 40%, 45%, 50%, 55%, 60%, 65%, 70%, 75%, 80%, 85%, 90%, or 95%, of cells in the composition express a gamma-globin protein. Each cell within the composition of cells according to this aspect may have at least one modified allele. Cells according to this aspect may be derived from a subject that does not carry said T→C mutation. Each cell within said composition of cells according to this aspect may have at least one modified allele and/or may have an average of 1.0 to 2.0 modified alleles per cell. Cells according to this aspect may be selected from the group consisting of a CD34+ hematopoietic stem and progenitor cells (CD34+ HSPCs) and cells in the erythroid lineage. Cells according to this aspect may be derived from the group consisting of bone marrow. peripheral blood. mobilized peripheral blood. cord blood. and induced pluripotent stem cells (iPSCs).

Another aspect of this disclosure concerns a method of treating a subject which includes administering to the subject a composition of cells, wherein a plurality of cells of the composition comprise at least one synthetic allele of an HBG1 or HBG2 gene, the synthetic allele comprising a T→C mutation at −175 base pairs upstream of a 5′ UTR (i.e., 228 bp (−228) upstream from transcription start site) of the HBG1 or HBG2 gene. Erythrocyte lineage cells derived from this plurality of cells can express a gamma-globin protein for a period of 30, 60, 90, 120, 180, 360 or more days following administration of the composition to the subject. In some cases, this plurality of cells comprises or constitutes fewer than 50%, 40% or 30% of cells of the composition.

In still another aspect, this disclosure relates to a guide RNA molecule comprising a targeting domain comprising a nucleotide sequence complementary or partially complementary with a target sequence encompassing or proximal to position −175 of a 5′ UTR and/or −228 of a transcription start site of HBG1 or HBG2 gene; the target sequence may be between −150 and −250 of the proximal promoter region −175 and/or it may be configured to guide a nucleobase modification at the −175/−228 position. The nucleobase modification can result in a T→C mutation in some embodiments.

Another aspect of this disclosure encompasses nucleic acid compositions comprising the guide RNA molecules described in a foregoing aspect of the disclosure and optionally including a sequence encoding a base editing protein or peptide. According to this aspect, the base editing protein or peptide may be an adenine base editor (ABE). In some aspects the ABE may be selected from the group consisting of ABE7.10, ABE8e, and an ABE comprising SpRY. In some aspects, the base editing protein or peptide in turn may comprise an adenosine deaminase, such as ABE7.10, ABEmax, etc. These components may be configured, individually or collectively, to introduce a T→C mutation in or near the target sequence of the guide RNA. In some embodiments, the nucleic acid composition is a viral vector genome or an mRNA.

One aspect of this disclosure relates to an isolated ribonucleoprotein complex comprising the guide RNA and base editing protein or peptide components described above.

A method of increasing expression of fetal hemoglobin in a subject in need thereof is addressed by another aspect of this disclosure. The method may include contacting a cell of the subject with a base editing system configured to induce, on a percentage basis, at least 0.75× or at least 1× fetal hemoglobin induction for each x percent base editing achieved. Alternatively or additionally, the method utilizes a base editing system configured to introduce a T→C mutation at a position 175 bp (−175) upstream from a 5′ UTR region or 228 bp (−228) upstream from a transcription start site of the HBG1 or HBG2 gene. The chromosomal coordinates of these sites are HBG2: GRCh/hg 19; chr11:5,276,186.; and HBG1: GRCh/hg19; chr11:5,271,262.

The percentage of base editing can be measured in any aspect of this disclosure as the ratio of reads comprising the T→C relative to the total number of reads achieved by deep sequencing of an amplicon comprising at least a portion of an HBG1 or HBG2 upstream promoter region. Similarly. the percentage of fetal hemoglobin induction is measured by at least partially differentiating a cell or composition of cells into an erythroid lineage and quantitatively determining at least one of:
a fraction of hemoglobin tetramers comprising a gamma-globin; or
a fraction of gamma-globin monomers relative to total non-alpha monomers present. This determination may be performed by, e.g., liquid chromatography (e.g., using ion exchange or reverse phase chromatography media), and by spectroscopic quantitation at 220 nm and 415 nm.

An additional aspect of this disclosure is addressed to a method of altering a cell, comprising creating a TAL1 binding site in a proximal upstream promoter region of one or more of an HBG1 gene and an HBG2 gene, e.g., by introducing a T→C mutation at one or more of:
HBG2: GRCh/hg 19; chr11:5,276, 186.; and
HBG1: GRCh/hg19; chr11:5,271,262.

These coordinates may be expressed. alternatively or additionally, as position 175 bp (−175) upstream from 5′ UTR region or 228 bp (−228) upstream from transcription start site of the HBG1 or HBG2 gene, relative to the proximal promoter sequence 5′-CTATCTCAATGCAAATATCT-3′ (SEQ ID NO: 12).

Another aspect of this disclosure concerns a cell derived from a subject, comprising at least one synthetic TAL1 binding site in a proximal upstream promoter region of one or more of an HBG1 gene and an HBG2 gene. The cell may be characterized in that a differentiated cell in the erythrocyte lineage derived therefrom expresses a quantity of g-globin protein greater than the quantity of g-globin protein expressed by a cell in the erythrocyte lineage derived from the same subject which lacks the at least one synthetic TAL1 binding site.

Still another aspect of the disclosure includes a composition of cells, wherein at least 20%, 25%, 30%, 35%, 40%, 45%, 50%, 55%, 60%, 65%, 70%, 75%, 80%, 85%, 90%, 95%, or more alleles of HBG1 and HBG2 genes within said composition of cells comprise a synthetic TAL1 binding site in a proximal promoter region of the HBG1 or HBG2 gene. In some embodiments, at least 20%, 25%, 30%, 35%, 40%, 45%, 50%, 55%, 60%, 65%, 70%, 75%, 80%, 85%, 90%, or 95%, of cells in the composition express a gamma-globin protein when differentiated into an erythrocyte lineage. Alternatively, or additionally, at least 20%, 25%, 30%, 35%, 40%, 45%, 50%, 55%, 60%, 65%, 70%, 75%, 80%, 85%, 90%, or 95%, of hemoglobin produced by the cells comprises a gamma-globin protein.

Yet another aspect of this disclosure is directed to a synthetic allele comprising one or more of a KLF1 transcriptional activator binding site at a T→C mutation at a position −198 base pairs of the promoter region of the HBG1 or HBG2 gene, a TAL1 transcriptional activator binding site at a T→C mutation at a position −175 base pairs of the promoter region of the HBG1 or HBG2 gene, and a GATA1 transcriptional activator binding site at a T→C mutation at a position −113 base pairs of the promoter region of the HBG1 or HBG2 gene.

Still another aspect of this disclosure is directed to a cell derived from a subject having a hemoglobinopathy, the cell comprising at least one synthetic allele of an HBG1 or HBG2 gene, said synthetic allele comprising a T→C mutation at a position selected from the group consisting of: a position −198 base pairs of the promoter region of the HBG1 or HBG2 gene, a position −175 base pairs of the promoter region of the HBG1 or HBG2 gene, and a position −113 base pairs of the promoter region of the HBG1 or HBG2 gene.

Still a further aspect of this disclosure is directed to a composition of cells, wherein at least 20%, 25%, 30%, 35%, 40%, 45%, 50%, 55%, 60%, 65%, 70%, 75%, 80%, 85%, 90%, 95%, or more alleles of HBG1 and HBG2 genes within said composition of cells comprise a T→C mutation at a position selected from the group consisting of: a position −198 base pairs of the promoter region of the HBG1 or HBG2 gene, a position −175 base pairs of the promoter region of the HBG1 or HBG2 gene, and a position −113 base pairs of the promoter region of the HBG1 or HBG2 gene.

An additional aspect of this disclosure is directed to a method of treating a subject. comprising administering to the subject a composition of cells, wherein a plurality of cells of the composition comprise at least one synthetic allele of an HBG1 or HBG2 gene, the synthetic allele comprising a T→C mutation at a position selected from the group consisting of: a position −198 base pairs of the promoter region of the HBG1 or HBG2 gene, a position −175 base pairs of the promoter region of the HBG1 or HBG2 gene, and a position −113 base pairs of the promoter region of the HBG1 or HBG2 gene.

Cells and compositions of cells, as well as guide RNAs, ribonucleoprotein complexes, and nucleic acid compositions according to the foregoing aspects of the disclosure are useful in therapy. such as the treatment of hemoglobinopathies including anemia, beta-thalassemia, and sickle cell disease, for example. Each of these aspects may be adapted into methods of treating subjects by administration of the same.

The foregoing listing is intended to be exemplary and non-limiting. Additional aspects and embodiments of this disclosure are presented in the detailed description that follows.

### BRIEF DESCRIPTION OF THE DRAWINGS

FIG. 1(a-i): The A base editor ABE7.10 generates HPFH variants and induces HbF in HUDEP-2 erythroid cells. FIG. 1a: The HPFH variants (red nucleotides) are shown as excerpts from the proximal promoter region of the gamma-globin genes (HBG1 and HBG2). Protospacers and protospacer-adjacent motifs (PAMs) are indicated below each excerpt for the installation of −198 T→C, −175 T→C and −113 A→G. Potential bystander nucleotides for ABE7.10 are indicated in blue. The position of the substrate nucleotides in the protospacer is indicated by numbered superscripts counting from the 5′ end of the protospacer. Proximal promoter region showing HPFH variants −198 T→C, −175 T→C and −113 A→G. Single guide (sg) RNAs (sgRNAs) were generated to target targeting each indicated region are shown below. Base positions within the editing window are labeled from 5′ to 3′ with the target adenosines for creating each variant shown in red and potential bystander adenosines in blue. Binding motifs for the repressor proteins BCL11A and ZBTZ7A are boxed. Cells were electroporated with ABE 7.10 mRNA (2 μg) and sgRNA (2 μg), transferred to culture medium that promotes erythroid maturation, and analyzed as indicated in each panel. Control (Ctrl) cells were processed in parallel, but not electroporated (SEQ ID NO: 18, SEQ ID NO: 19, SEQ ID NO: 20, SEQ ID NO: 21, SEQ ID NO: 22, and SEQ ID NO: 23). FIG. 1b, FIG. 1c, and FIG. 1d: Base editing frequencies measured by targeted deep sequencing at 72 hours post-electroporation. The efficiency of the target edit is indicated in red and bystander edits in blue. Bar graphs show on-target mutations in red and bystander mutations in blue. FIG. 1e: The % HbF protein determined by ion-exchange high-performance liquid chromatography (HPLC) of 10 day cell lysates extracted after 10 days of erythroid maturation. FIG. 1f: HUDEP-2 cells containing only a single beta-globin locus containing HBG1 and HBG2 (and a deletion of all beta-globin genes on the other allele) were edited as described in panel A followed by isolation of individual clones with defined mutations. In each case. these mutations were identical in both the HBG1 and HBG2 promoters. The % HbF in cell lysates of clones with the indicated mutations were determined by ion-exchange HPLC. Each datapoint represents an individual clone. FIG. 1g: Frequency of indels measured by targeted deep sequencing of PCR fragments generated around the indicated sites at 72 hours post-electroporation. FIG. 1h: Cells immunostaining immune stained for fetal hemoglobin F (F-cells) measured by flow cytometry at culture day 10 in maturation medium after culture for 10 days in erythroid maturation medium. FIG. 1i: Schematic diagram showing the hemizygous HUDEP-2 cell line harboring single copy of beta-like globin genes used for clonal analysis. Paired sgRNAs were used make a large deletion in beta-globin locus to remove beta-like globin genes (doted lines) and the clones which had single copy of globin genes were used for the study. Bar graphs show mean values standard deviation (SD). ****P≤0.0001. ***P≤0.001. **P≤0.01 and *P≤0.05 (unpaired t test).

FIG. 2(a-h): ABE7.10 generates HPFH variants in healthy donor CD34+ cells and induces HbF in their erythroid progeny generated in vitro. G-CSF-mobilized CD34+ cells were edited by electroporation with ribonucleoprotein (RNP) consisting of ABE 7.10 (100 pmol)+sgRNA (300 pmol) using the Lonza 4D Transfection System with ribonucleoprotein (RNP) consisting of ABEmax 7.10 (100 pmol)+sgRNA (300 pmol), then incubated cultured for 21 days in crythroid differentiation medium to support erythroid differentiation. Negative controls included samples that were not electroporated (NE) and samples electroporated with an editor complexed with a non-targeting guide (NT). Non-targeting (NT) sgRNAs were used as negative control and control (Ctrl) cells were processed in parallel, but not electroporated. Data show studies from 2 biological replicates from 3 different CD34+ cell donors (n=6) with each donor indicated by a different color. FIG. 2a. FIG. 2b, and FIG. 2c: Base editing frequencies at the indicated adenosines measured by targeted deep sequencing 72 hours post-electroporation. FIG. 2d: Frequency of indels measured by targeted deep sequencing of PCR fragments products generated around the indicated sites at 72 hours post-electroporation. FIG. 2e: F-cells measured by flow cytometry after 21 days of crythroid differentiation and maturation. FIG. 2f: The % HbF protein in day 21 erythroid cell lysates determined by ion-exchange HPLC. FIG. 2g: F-cells measured by flow cytometry at culture day 21. FIG. 2h: Erythroid maturation markers measured by immuno-flow cytometry at culture day 14. Representative plots show the expression of CD49d and Band3 on CD235a+ erythroid cells. Bar graphs show mean values SD. **** P≤0.0001. ***P≤0.001 and *P≤0.5 (unpaired t test). ns, not significant.

FIG. 3(a-i): ABE7.10 generates HPFH −175 T→C in SCD donor CD34+ cells and induces HbF in erythroid progeny to inhibit their hypoxic sickling. Plerixafor mobilized CD34+ cells from three different SCD donors were edited at the −175 HPFH site as described for main FIG. 2a. then incubated for 21 days in medium to support erythroid differentiation. Control (Ctrl) cells were processed in parallel. but not electroporated. FIG. 3a: Base editing frequencies at 3, 6- and 9-days post-electroporation. FIG. 3b: F-cells measured by flow cytometry at culture day 21. FIG. 3c: The % HbF protein in day 21 erythroid cell lysates measured by ion exchange HPLC. FIG. 3d: Erythroid maturation markers measured by immuno-flow cytometry at culture day 14. Representative plots show the expression of CD49d and Band3 on CD235a+ erythroid cells. FIG. 3e: Flow cytometry-purified human reticulocytes from recipient bone marrow were incubated for 8 hours in 2% oxygen and visualized by phase-contrast microscopy using the IncuCyte S3 Live-Cell Analysis System (Sartorius) with a 20× objective. FIG. 3f: Quantification of sickle cell fraction from data shown in panel e. More than 300 cells from at least 3 different fields were scored for each condition. FIG. 3g: Indel frequencies on days 3, 6- and 9-days post electroporation. FIG. 3h: F-cells measured by flow cytometry at culture day 21. FIG. 3i: Enucleated (Hoechst−) human CD235a+ erythroblasts in mouse bone marrow at culture day 21. Bar graphs show mean values SD. ***P≤0.001. **P≤0.01 (unpaired t test). ns-not significant.

FIG. 4(a-k): ABE7.10 generates HPFH mutations in healthy human hematopoietic stem cells (HSCs). G-CSF-mobilized CD34+ cells were edited electroporated with ABEmax7.10max, as described in main FIG. 2a, then transplanted into NBSGW mice via tail-vein injection (0.5 million cells/mouse). Non-targeting (NT) sgRNAs were used as negative control and control (Ctrl) cells were processed in parallel, but not electroporated. Data show studies from 9 total mice transplanted with cells from 2 different CD34+ cell donors indicated by a different color. FIG. 4a and FIG. 4b: Base editing frequencies at 72 hours post-electroporation (Input HSPCs) and 18 weeks post-transplantation. Human genomic DNA was sequenced from the in human cells within whole bone marrow (BM) extracted from mice, and this population was further sorted into bone marrow-derived CD34+ HSPCs and human CD235a+ erythroid cells. FIG. 4c: Normalized human chimerism in recipient bone marrow, shown as the percentage of human (h) CD45+ cells. FIG. 4d: Reconstitution of human B (CD19+), myeloid (CD33+) and T (CD3+) cells shown as percentages of the human CD45+ population in bone marrow at 18 weeks. FIG. 4e: Erythroid chimerism shown as the percentage of human CD235a+ cells within the total human CD45—population (mouse and human). FIG. 4f: Percentage of F-cells within the hCD235a+ crythroid population in bone marrow. FIG. 4g: The % HbF protein in human CD235a+ erythroid cells isolated from recipient bone marrow. FIG. 4h: The % HbF as a function of the base editing percentage from the values plotted in panels a, b and g. FIG. 4i: Frequency of indels measured by targeted deep sequencing for respective sites in the indicated cell populations, three days after electroporation (Input HSPCs) or in cells extracted from mice 18 weeks after transplantation. FIG. 4j: Representative flow cytometry plots showing the percentage of F-cells within the hCD235a+ erythroid population in bone marrow. FIG. 4k: Erythroid maturation markers measured by immuno-flow cytometry at culture. Mean values SD are shown from 9 total mice transplanted with cells from 2 different healthy CD34+ cell donors. ****P≤0.0001 (unpaired t test). ns, not significant.

FIG. 5(a-m): ABE7.10 generates the −175 T→C HPFH mutation in SCD-donor derived HSCs. Plerixafor-mobilized CD34+ cells an SCD donor were edited with ABE7.10 RNP targeting the −175 site, as described for FIG. 2(a-h), then transplanted into NBSGW mice via tail-vein injection (0.5 million cells/mouse). Controls (Ctrl) donor cells were processed in parallel, but not electroporated. Each dot in the graphs represents a separate mouse with individual CD34+ cell donors represented by different colors. FIG. 5a: Base editing frequencies at 72 hours post-electroporation (Input HSPCs) and 18 weeks post-transplantation in human cells within whole bone marrow (BM) and bone marrow-derived CD34+ HSPCs, CD19+ B-cells. CD33+ myeloid cells and hCD235a+ erythroid cells. FIG. 5b: Normalized human chimerism in bone marrow. shown as the percentage of hCD45+ cells. FIG. 5c: Reconstitution of human B, myeloid and T cells, shown as percentages of the human CD45+ population in bone marrow at 16 weeks. FIG. 5d: Erythroid chimerism shown as the percentage of human CD235a+ cells within the total human CD45—population (mouse and human). FIG. 5c: The % HbF protein in human CD235a+ erythroblasts isolated from recipient bone marrow. FIG. 5f: Magnetic-activated cell sorting (MACS)-purified human reticulocytes from recipient bone marrow were incubated for 8 hours in 2% oxygen and visualized by phase-contrast microscopy. FIG. 5g: Quantification of sickle cell fraction from data shown in panel f. More than 300 cells from at least 3 different fields were scored for each condition. FIG. 5h: Indel frequencies in input HSPCs and in the indicated human donor cell populations at 16 weeks post-transplantation. FIG. 5i: Erythroid maturation markers CD49a and Band3 on hCD235a+ erythroid cells in recipient bone marrow. FIG. 5j: Enucleated (Hoechst⁻) human CD235a+ erythroblasts in recipient bone marrow. FIG. 5k: Percentage of F-cells within human CD235a+ cells in recipient bone marrow. FIG. 5l: Representative flow cytometry plots showing the % F-cells cells on the hCD235a+ erythroid fraction of bone marrow. FIG. 5m: The % HbF as a function of the base editing percentage from the values plotted in FIG. 5a and FIG. 5c. Mean values SD are shown from 3 total mice. ***P≤0.001. **P≤0.01 (unpaired t test). ns-not significant.

FIG. 6(a-i): The A base editor ABE7.10 generates HPFH variants and induces HbF in HUDEP-2 erythroid cells. FIG. 6a: The HPFH variants (red nucleotides) are shown as excerpts from the proximal promoter region of the y-globin genes (HBG1 and HBG2). Protospacers and protospacer-adjacent motifs (PAMs) are indicated below each excerpt for the installation of-198 T→C, −175 T→C and −113 A→G. Potential bystander nucleotides for ABE7.10 are indicated in blue. The position of the substrate nucleotides in the protospacer is indicated by numbered superscripts counting from the 5′ end of the protospacer. Single guide RNAs (sgRNAs) were generated to target each indicated region binding motifs for the repressor proteins BCL11A and ZBTZ7A are boxed in blue. Cells were electroporated with ABE 7.10 mRNA (2 μg) and sgRNA (2 μg). transferred to culture medium that promotes erythroid maturation. and analyzed as indicated in each panel. Untreated cells were processed in parallel. but not electroporated (SEQ ID NO: 18, SEQ ID NO: 19, SEQ ID NO: 20, SEQ ID NO: 21, SEQ ID NO: 22, and SEQ ID NO: 23). FIG. 6b. FIG. 6c. and FIG. 6d: Base editing frequencies measured by targeted deep sequencing at 72 hours post-electroporation. The efficiency of the target edit is indicated in red and bystander edits in blue. FIG. 6e: The % HbF protein determined by ion-exchange high-performance liquid chromatography (HPLC) of cell lysates extracted after 10 days of erythroid maturation. FIG. 6f: Hemizygous HUDEP-2 cells containing only a single γ-globin locus containing HBG1 and HBG2 (see FIG. 6i) were edited as described in panel A followed by isolation of individual clones with defined mutations. The % HbF in cell lysates of clones with the indicated mutations were determined by ion-exchange HPLC. Each datapoint represents an individual clone. Bar graphs show mean values±standard deviation (SD). ****P≤0.0001 and *P≤0.05 (unpaired t test). FIG. 6g: Frequency of indels measured by targeted deep sequencing of PCR fragments generated around the indicated sites at 72 hours post-electroporation. Each data point represents a biological replicate experiment. Bar graphs show mean values with error bars indicating standard deviation (SD) FIG. 6h: Cells immune-stained for fetal hemoglobin (F-cells) measured by flow cytometry after culture for 10 days in erythroid maturation medium. FIG. 6i: Schematic showing the hemizygous HUDEP-2 cell line harboring single copy of b-like globin genes used for clonal analysis. Paired sgRNAs were used make a large deletion in b-globin locus to remove b-like globin genes (doted lines) and the clones which had single copy of globin genes were used for the study. Mean values±SD are shown from 3 biological replicate experiments. ****P≤0.0001 and *P≤0.05 (unpaired t test).

FIG. 7(a-h): The −175T→C mutation creates a de novo binding site for TAL1 and requires GATA1 for active binding. FIG. 7a: TAL1 CUT & RUN analysis at the b-like globin gene cluster in hemizygous HUDEP-2 clone with −175 T→C at both HBG1 and HBG2 promoter. The HBG1/2 gene along with promoters within the b-globin cluster were highlighted in blue. HUDEP-1 cells were processed along with HUDEP-2 cells as a reference control. FIG. 7b: CUT&RUN footprint analysis of TAL1 peaks at HBG1 2 promoter in hemizygous HUDEP-2 clones with −175 T→C. Consensus TAL1 binding motifs are highlighted in orange. The GATA motif proximal to TAL1 binding site were highlighted in brown. The wild type bases and −175 T→C base that creates TAL1 binding site were illustrated (SEQ ID NO: 24 and SEQ ID NO: 25). FIG. 7c: Genome-wide targeted motif footprint analysis of CUT&RUN data showing cut probability of each base surrounding and within TAL1-binding CAGATG motif FIG. 7d: CUT & RUN analysis TAL1 (red peaks) and GATA1 (blue peaks) at the b-like globin gene cluster in wild type HUDEP-2 clone and double mutants. The HBG1/2 gene along with promoters within the b-globin cluster were highlighted in blue. The HbF was measured by ion-exchange HPLC shown in brackets of each HUDEP-2 clones. FIG. 7c: Hemizygous HUDEP-2 clones with −175 T→C and −187/−189 clones were generated by electroporation of ABE7.10+sgRNA targeting GATA motif proximal to TAL1 binding site. TAL1 and GATA motif are highlighted in blue (SEQ ID NO: 26, SEQ ID NO: 27, and SEQ ID NO: 28). FIG. 7f: The % HbF in cell lysates of clones with the indicated mutations were determined by ion-exchange HPLC. FIG. 7g: Genome-wide targeted motif footprint analysis of GATA1 CUT&RUN data showing cut probability of each base surrounding and within TAL1-binding CAGATG motif and GATA motif. FIG. 7h: NFYA CUT & RUN analysis at the b-like globin gene cluster in wild type HUDEP-2, −175 T→C and double mutants clones. The HBG1/2 gene along with promoters within the b-globin cluster were highlighted in blue. The HbF was measured by ion-exchange shown in brackets of each HUDEP-2 clones. ****P≤0.0001 (unpaired t test).

FIG. 8(a-h): ABE7.10 generates HPFH variants in healthy donor CD34⁺ cells and induces HbF in their erythroid progeny generated in vitro. G-CSF-mobilized CD34⁺ cells were edited by electroporation with ribonucleoprotein (RNP) consisting of ABE 7.10 (100 pmol)+sgRNA (300 pmol) using the Lonza 4D Transfection System, then cultured for 21 days in crythroid differentiation medium. Data show studies from 2 biological replicates from 3 different CD34⁺ cell donors (n=6) with each donor indicated by a different color. FIG. 8a, FIG. 8b, and FIG. 8c: Base editing frequencies at the indicated adenosines (see FIG. 6a) measured by targeted deep sequencing 72 hours post-electroporation. FIG. 8d: F-cells measured by flow cytometry at culture day 21. Mean values±SD are shown from 6 biological replicate experiments using CD34+ cells from three different healthy donors. FIG. 8c: The % HbF protein in day 21 erythroid cell lysates determined by ion-exchange HPLC. Bar graphs show mean values±SD. ****P<0.0001 and ***P≤0.001 and *P≤0.5 (unpaired t test). ns, not significant. FIG. 8f: Frequency of indels measured by targeted deep sequencing of PCR products generated around the indicated sites at 72 hours post-electroporation. FIG. 8g: Representative flow cytometry plots showing the percentage of F-cells measured by flow cytometry at culture day 21. FIG. 8h: Erythroid maturation markers measured by immuno-flow cytometry at culture day 14. Representative plots show the expression of CD49d and Band3. Mean values±SD are shown from 6 biological replicate experiments using CD34+ cells from three different healthy donors. Bar graphs show mean values±SD. ****P≤0.0001 (unpaired t test). ns, not significant.

FIG. 9(a-m): Clonal analysis of HPFH mutants erythroid progeny edited with ABE7.10. G-CSF-mobilized CD34⁺ cells were edited by electroporation with ribonucleoprotein (RNP) consisting of ABE 7.10 (100 pmol)+sgRNA (300 pmol) using the Lonza 4D Transfection System. then edited cells were cultured in methocult media that supports erythroid differentiation and individual clones were picked for HbF and genotype analysis. FIG. 9a: Base editing frequencies at 72 hours post-electroporation in bulk edited cells. FIG. 9b: % on-target edits in bulk edited cells. FIG. 9c: The % HbF protein in day 21 erythroid cell lysates determined by ion-exchange HPLC in bulk edited cells. FIG. 9d and FIG. 9f: The % HbF protein in individual clones with number of HBG1/2 genes with HPFH variants. FIG. 9g. FIG. 9h, and FIG. 9i: The % HbF as a function of the base editing percentage for HPFH variants from the values plotted in FIG. 9(d-i). Each dot represents individual clones. FIG. 9j: Frequency of indels measured by targeted deep sequencing in the indicated locus, three days after electroporation. FIG. 9k: The % HbF protein in day 21 erythroid cell lysates determined by ion-exchange HPLC in bulk edited cells. FIG. 9l and FIG. 9m: The % HbF as a function of the base editing percentage for HPFH like variants in individual clones for indicated locus. Each dot represents individual clones.

FIG. 10(a-j): ABE7.10 generates −175 T→C HPFH mutation in healthy human hematopoietic stem cells (HSCs). G-CSF-mobilized CD34⁺ cells were electroporated with ABE7.10 then transplanted into NBSGW mice via tail-vein injection (0.5 million cells/mouse). Data show studies from 9 total mice transplanted with cells from 2 different CD34⁺ cell donors indicated by a different color. FIG. 10a: Base editing frequencies at 72 hours post-electroporation (Input HSPCs) and 18 weeks post-transplantation. Human genomic DNA was sequenced from the whole bone marrow (BM) extracted from mice. and this population was further sorted into CD34⁺ HSPCs and human CD235a⁺ erythroid cells. FIG. 10b: Normalized human chimerism in recipient bone marrow, shown as the percentage of human (h) CD45+ cells. FIG. 10c: Reconstitution of human B (CD19⁺), myeloid (CD33⁺) and T (CD3⁺) cells shown as percentages of the human CD45⁺ population in bone marrow at 18 weeks. FIG. 10d: Erythroid chimerism shown as the percentage of human CD235a⁺ cells within the total human CD45⁻ population (mouse and human). (e: Percentage of F-cells within the hCD235a⁺ erythroid population in bone marrow. FIG. 10f: The % HbF protein in human CD235a⁺ crythroid cells isolated from recipient bone marrow. FIG. 10g: The % HbF as a function of the base editing percentage from the values plotted in panels a and f. ****P≤0.0001 (unpaired t test). ns, not significant. BM-Bone Marrow. FIG. 10h: Frequency of indels measured by targeted deep sequencing in the indicated cell populations. three days after electroporation (Input HSPCs) or in cells extracted from mice 18 weeks after transplantation. FIG. 10ib: Erythroid maturation markers CD49a and Band3 on hCD235a+ erythroid cells in recipient bone marrow. FIG. 10j: Representative flow cytometry plots showing the % F-cells cells on the hCD235a+ crythroid fraction of bone marrow. ****P≤0.0001 (unpaired t test). UT-Untreated. ns, not significant. BM-Bone Marrow.

FIG. 11(a-m): ABE7.10 generates the −175 T→C HPFH mutation in SCD-donor derived HSCs. Plerixafor-mobilized CD34⁺ cells an SCD donor were edited with ABEmax7.10 RNP targeting the −175 site, then transplanted into NBSGW mice via tail-vein injection (0.5 million cells/mouse). Data show studies from total 11 mice transplanted with cells from 3 different SCD donors indicated by a different color. Each dot in the graphs represents a separate mouse. FIG. 11a: Base editing frequencies at 72 hours post-electroporation (Input HSPCs) and 16 weeks post-transplantation in human cells within whole bone marrow (BM) and bone marrow-derived CD34⁺ HSPCs. CD19⁺ B-cells, CD33⁺ myeloid cells and hCD235a⁺ erythroid cells. FIG. 11b: Percentage of F-cells within human CD235a+ cells in recipient bone marrow. FIG. 11c: The % HbF protein in human CD235a+ erythroblasts isolated from recipient bone marrow. FIG. 11d: The % HbF as a function of the base editing percentage from the values plotted in panel a and c. FIG. 11e: Magnetic-activated cell sorting (MACS)-purified human reticulocytes from recipient bone marrow were incubated for 8 hours in 2% oxygen and visualized by phase-contrast microscopy. FIG. 11f: Quantification of sickle cell fraction from data shown in panel e. More than 300 cells from at least 3 different fields were scored for each condition. Bar graphs show mean values±SD. ****P≤0.0001 (unpaired t test). BM-Bone Marrow. FIG. 11g: Indel frequencies in input HSPCs and in the indicated human donor cell populations at 16 weeks post-transplantation. FIG. 11h: Normalized human chimerism in bone marrow. shown as the percentage of hCD45+ cells. FIG. 11i: Reconstitution of human B, myeloid and T cells, shown as percentages of the human CD45⁺ population in bone marrow at 16+ weeks. FIG. 11j: Erythroid chimerism shown as the percentage of human CD235a⁺ cells within the total human CD45⁻ population (mouse and human). FIG. 11k: The % g-globin chain variant analysis by reverse-phase HPLC. FIG. 11l: Erythroid maturation markers CD49a and Band3 on hCD235a+ crythroid cells in recipient bone marrow. FIG. 11m: Enucleated (Hoechst⁻) human CD235a+ erythroblasts in recipient bone marrow.

FIG. 12(a-f). ABE8e mediated base editing of HBG1/2 promoter improved the editing efficiency and HbF induction in their erythroid progeny generated in vitro. G-CSF-mobilized CD34+ cells were edited by electroporation with ribonucleoprotein (RNP) consisting of ABE8e (100 pmol)+sgRNA (300 pmol) using the Lonza 4D Transfection System,, then cultured for 21 days in erythroid differentiation medium. Data show studies from 6 biological replicates. Cells were collected at 0, 6, 12, 24 and 48 hours post electroporation and measured for CDKN1 expression. Cells were collected on day 3 and 6 post electroporation for measuring editing frequencies and indels respectively. FIG. 12a: Base editing frequencies at the indicated adenosines (see FIG. 11a) measured by targeted deep sequencing 72 hours post-electroporation. FIG. 12b: Frequency of indels measured by targeted deep sequencing of PCR products generated around the indicated sites at 72 hours post-electroporation. FIG. 12c: The % HbF protein in day 21 erythroid cell lysates determined by ion-exchange HPLC. Bar graphs show mean values±SD. ****P≤0.0001 and ***P≤0.001 and **P≤0.01 (unpaired t test). FIG. 12d: editing frequency of indicated target site or locus on day 3 post electroporation. FIG. 12e: Digital droplet PCR (ddPCR) quantification of CDKN1 (p21) mRNA expression at different time points after base editing, normalized to RPP30 mRNA. FIG. 12f: 4.9 kb deletions generated by simultaneous double-stranded DNA breaks in the duplicated tandem HBG1 and HBG2 genes. Graphs show mean±SD from 3 biological replicate experiments. **** P≤0.0001 (unpaired t test). ns-not significant.

FIG. 13(a-b): Off-target base editing associated with ABE7.10 CD34+ hematopoietic stem and progenitor cells. CD34+ HSPCs treated with ABE7.10 protein (−175), or untreated controls (n=3). Note that the mutation frequency shown is summed across all reads with one or more A⋅T-to-G⋅C mutations the editing window. Using the CasOFFinder tool, 104 potential genomic off-target sites with 3 or fewer mismatches to the on-target HBG1/2 promoter sequence were identified and performed rhamp-seq to analyze the potential A→G conversion within 4-10 nucleotides of editing window opposite to PAM sequence. FIG. 13a: Forty-four off-targets with sensitivity of 0.2-0.9% reads with A→G conversion in edited compared to ctrl were shown. FIG. 13b: rhamp sequencing of 96 CIRCLE-seq predicted off-targets. Fifty-one off-targets with sensitivity of 0.2-0.9% reads with A→G conversion in edited compared to ctrl were shown. Bar graphs show the percentage of sequencing reads containing A⋅T-to-G⋅C mutations within protospacer positions 4-10 at on- and off-target sites in genomic DNA samples from healthy donor. Data shown as mean±SD.

FIG. 14(a-c): ABE8 PAM less SpCas9 (ABE8 SpRY) mediated base editing of HBG1/2 promoter in CD34+ cells and HbF induction in erythroid progeny as well as base editing using SpRY mRNA with different guides targeting for −175 site. G-CSF-mobilized CD34+ cells were edited by electroporation with ABE8 SpRY mRNA (2 μg)+sgRNA (2 μg) using the Lonza 4D Transfection System, then cultured for 21 days in erythroid differentiation medium. Data show studies from 2 biological replicates. FIG. 14a: Base editing frequencies at the indicated adenosines (see FIG. 6a) measured by targeted deep sequencing on day 6 post-electroporation. FIG. 14b: Frequency of indels measured by targeted deep sequencing of PCR products generated around the indicated sites on day 6 post-electroporation. FIG. 14c: The % HbF protein in day 21 erythroid cell lysates determined by ion-exchange HPLC. Bar graphs show mean values±SD. ****P≤0.0001 (unpaired t test). FIG. 14(d-e): 2 μg of SpRY mRNA+2 μg of gRNA used/0.5 million cells for each condition. FIG. 14d: Editing percentage of guides listed in Table 6. FIG. 14e: HbF percentage of guides listed in Table 6.

### DETAILED DESCRIPTION OF THE DISCLOSURE

### Definitions

Unless otherwise specified, each of the following terms has the meaning set forth in this section.

The indefinite articles “a” and “an” denote at least one of the associated noun, and are used interchangeably with the terms “at least one” and “one or more.” For example, the phrase “a module” means at least one module, or one or more modules.

The conjunctions “or” and “and/or” are used interchangeably.

A “base editor” is a system having nucleobase-modifying activity, however it may be delivered or implemented. Base editors are typically, but not necessarily, defined in terms of a mutation caused directly or indirectly by the activity of the system, e.g., a “T to C base editor” or an adenosine deaminase base editor. Base editors based on CRISPR systems have been described in the literature, including in PCT publication no. WO2020102659A1 to Liu, et al. at paragraphs [0004] through [0019], and US Patent Application Publication No. 20180073012A1 to Liu and Gaudelli, paragraphs [0002] through [0010], both of which are incorporated by reference herein for all purposes. Base editors used in the present disclosure are be delivered or implemented in any suitable manner; for example, when CRISPR-derived base editors are used they may be delivered as ribonucleoprotein complexes, as one or more modified or unmodified nucleic acids (e.g., mRNA), or as a nucleic-acid containing vector such as a viral vector (e.g., adenoviral, lentiviral, etc. vectors), a non-viral vector (e.g., a plasmid DNA), and the like. Additional delivery or implementation modalities are described in the literature and will be familiar to those of skill in the art.

“Domain” is used to describe a segment of a protein or nucleic acid. Unless otherwise indicated, a domain is not required to have any specific functional property.

An “indel” is an insertion and/or deletion in a nucleic acid sequence. An indel may be the product of the repair of a DNA double strand break, such as a double strand break formed by a genome editing system of the present disclosure. An indel is most commonly formed when a break is repaired by an “error prone” repair pathway such as the NHEJ pathway described in the literature. Indels are typically assessed by sequencing (most commonly by “next-gen” or “sequencing-by-synthesis” methods, though Sanger sequencing may still be used) and are quantified by the relative frequency of numerical changes (e.g., ±1, ±2 or more bases) at a site of interest among all sequencing reads. DNA samples for sequencing can be prepared by a variety of methods known in the art, and may involve the amplification of sites of interest by polymerase chain reaction (PCR) or the capture of DNA ends generated by double strand breaks, as in the GUIDEseq process described in Tsai 2016 (incorporated by reference herein). Other sample preparation methods are known in the art. Indels may also be assessed by other methods. including in situ hybridization methods such as the FiberComb™ system commercialized by Genomic Vision (Bagneux, France), and other methods known in the art.

“Replacement” or “replaced” as used herein with reference to a modification of a molecule does not require a process limitation but merely indicates that the replacement entity is present.

“Subject” means a human, mouse, or non-human primate. A human subject can be any age (e.g., an infant, child, young adult, or adult), and may suffer from a disease, or may be in need of alteration of a gene.

“Treat, ” “treating, ” and “treatment” as used herein mean the treatment of a disease in a subject (e.g., a human subject), including one or more of inhibiting the disease. i.e., arresting or preventing its development or progression; relieving the disease, i.e., causing regression of the disease state; relieving one or more symptoms of the disease; and curing the disease.

“Prevent, ” “preventing, ” and “prevention” as used herein means the prevention of a disease in a subject, e.g., in a human, including (a) avoiding or precluding the disease; (b) affecting the predisposition toward the disease; (c) preventing or delaying the onset of at least one symptom of the disease.

The terms “polynucleotide”, “nucleotide sequence”, “nucleic acid”, “nucleic acid molecule”, “nucleic acid sequence”, and “oligonucleotide” refer to a series of nucleotide bases (also called “nucleotides”) in DNA and RNA, and mean any chain of two or more nucleotides. The polynucleotides can be chimeric mixtures or derivatives or modified versions thereof, single-stranded or double-stranded. The oligonucleotide can be modified at the base moiety, sugar moiety, or phosphate backbone, for example, to improve stability of the molecule, its hybridization parameters, etc. A nucleotide sequence typically carries genetic information, including the information used by cellular machinery to make proteins and enzymes. These terms include double- or single-stranded genomic DNA, RNA, any synthetic and genetically manipulated polynucleotide, and both sense and antisense polynucleotides. This also includes nucleic acids containing modified bases.

Conventional IUPAC notation is used in nucleotide sequences presented herein (see also Cornish-Bowden 1985, incorporated by reference herein). It should be noted, however, that “T” denotes “Thymine or Uracil” insofar as a given sequence (such as a gRNA sequence) may be encoded by either DNA or RNA.

The terms “protein, ” “peptide” and “polypeptide” are used interchangeably to refer to a sequential chain of amino acids linked together via peptide bonds. The terms include individual proteins, groups or complexes of proteins that associate together, as well as fragments, variants, derivatives and analogs of such proteins. Peptide sequences are presented using conventional notation, beginning with the amino or N-terminus on the left, and proceeding to the carboxyl or C-terminus on the right. Standard one-letter or three-letter abbreviations may be used.

The term “synthetic allele” refers to an allele of a gene in the genetic material of a cell that is modified relative to one or both alleles of the same gene in the genetic material of a reference cell from the same subject. In one non-limiting example, the reference cell is a diploid germ-line cell from the same subject; in another non-limiting example. the reference cell is a cell taken from the subject prior to a therapeutic intervention according to this disclosure. Synthetic alleles may be created through gene editing techniques that are known in the art. and may differ genetically (e.g., in their nucleic acid sequence) and/or epigenetically (e.g., in their DNA methylation status, histone acetylation status, chromatin structure, or in other aspects that do not materially alter the coding sequence) from non-synthetic alleles of the same gene in the reference cell from the subject. Synthetic alleles may be modified in their coding and/or non-coding sequence(s). In various aspects of this disclosure, synthetic alleles may include genetically modified alleles of a human HBG1 and/or HBG2 gene.

### Overview

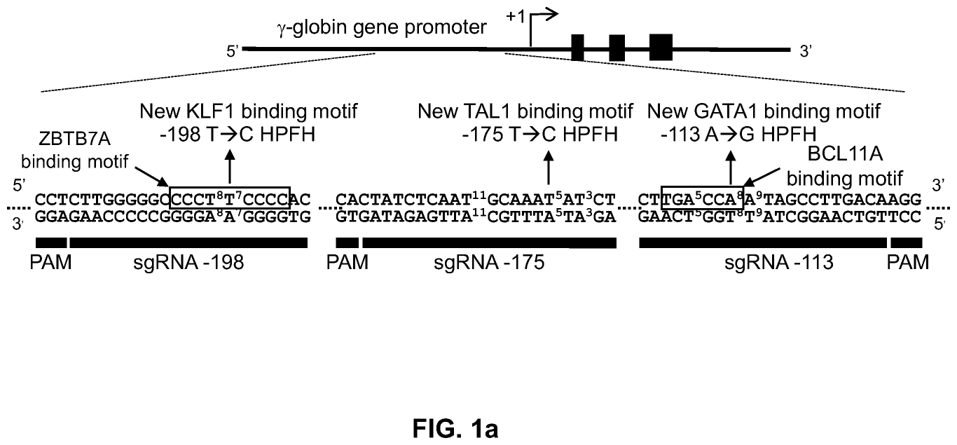

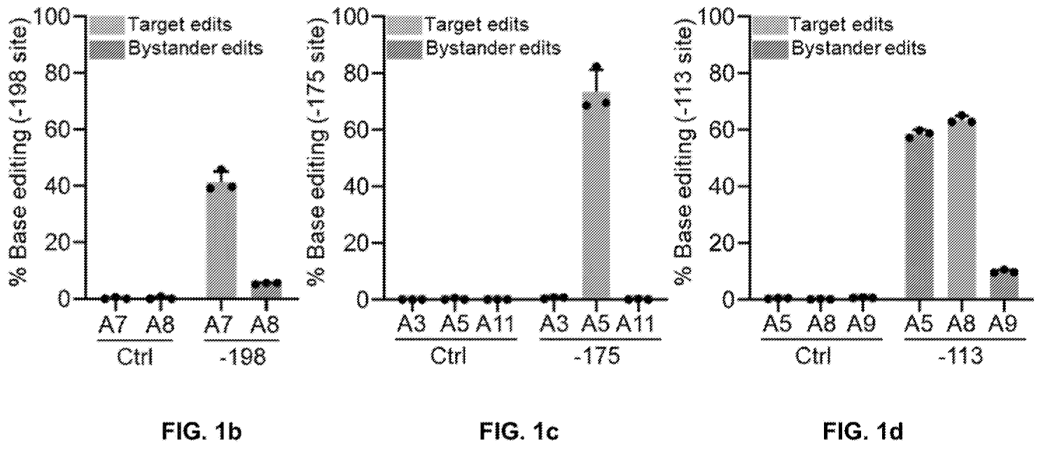

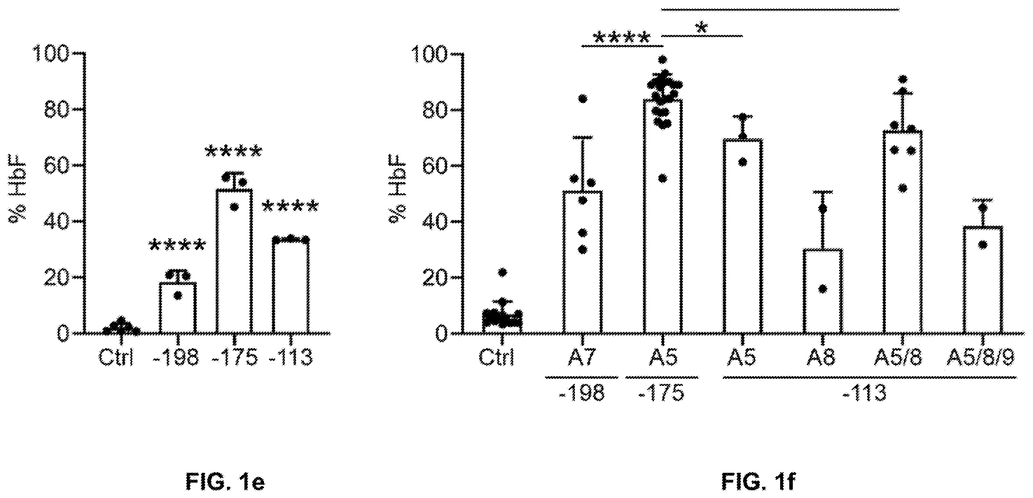

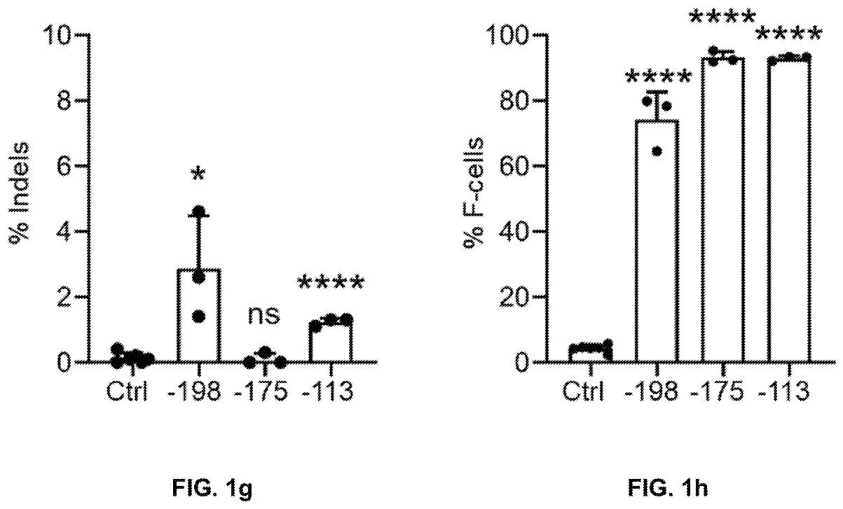

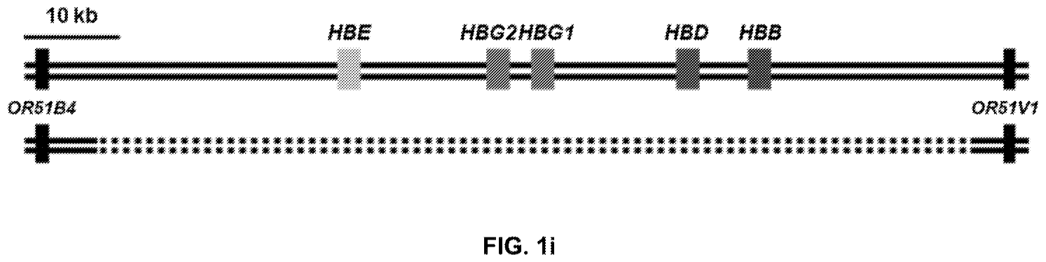

The present disclosure generally relates to the inventors' discovery that a synthetic T→C mutation in the −175 proximal promoter region of the HBG1 and HBG2 genes potentiates gamma-globin expression in HUDEP-2 cells and patient hematopoietic stem and progenitor cells (HSPCs) both in vitro and in vivo following transplantation of edited cells. FIG. 1a depicts the −214 to −108 proximal promoter region of HBG1 and HBG2, and illustrates two T→C mutations, at −198 and −175 and one A→G mutation, at −113, that are within the scope of the present disclosure. Each mutation is illustrated with reference to CRISPR-Cas9 base editor single-guide RNA targeting (sgRNA) and PAM sequences, but those of skill in the art will recognize that other suitable sgRNA sequences and PAM sequences within or proximate to these sites may be suitable for targeting of base editors or other genome editing systems in accordance with the embodiments of this disclosure.

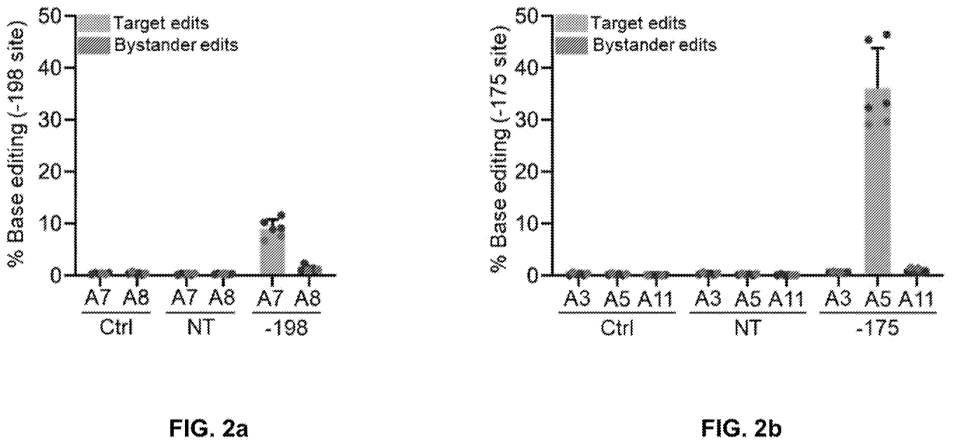

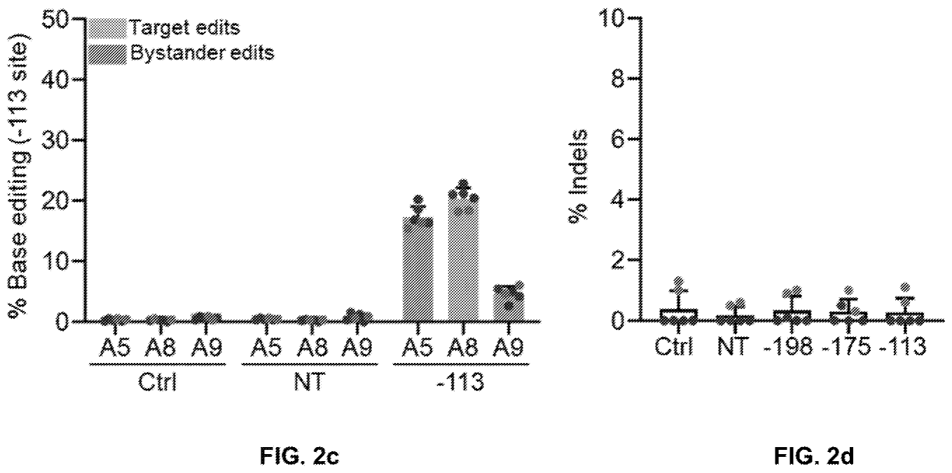

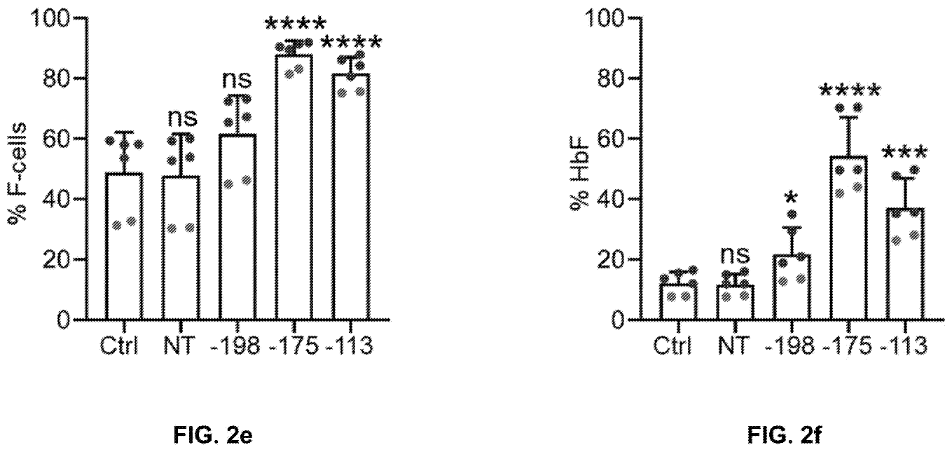

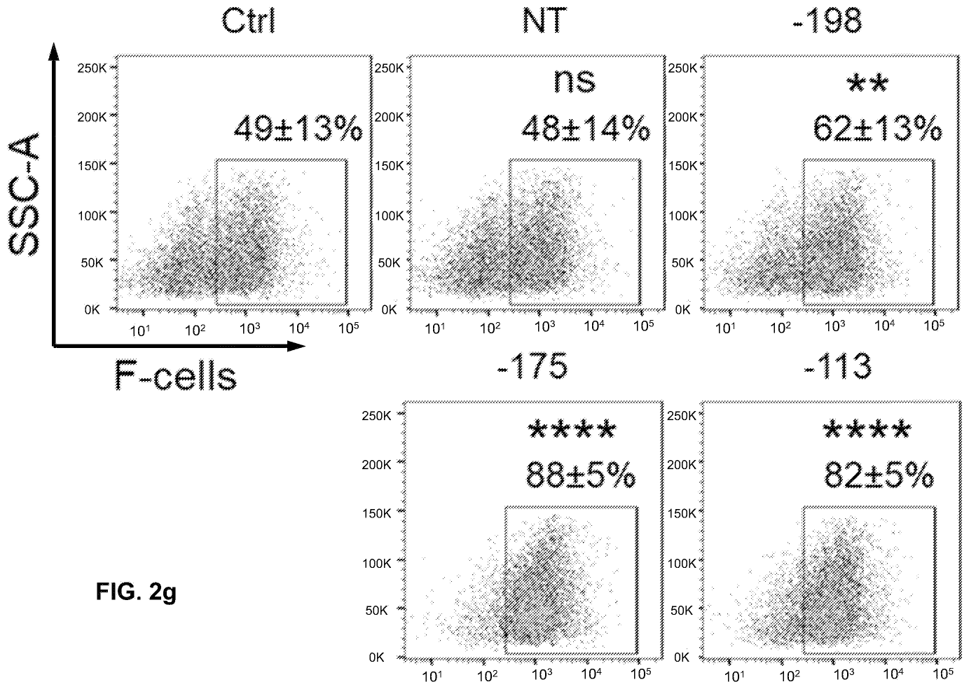

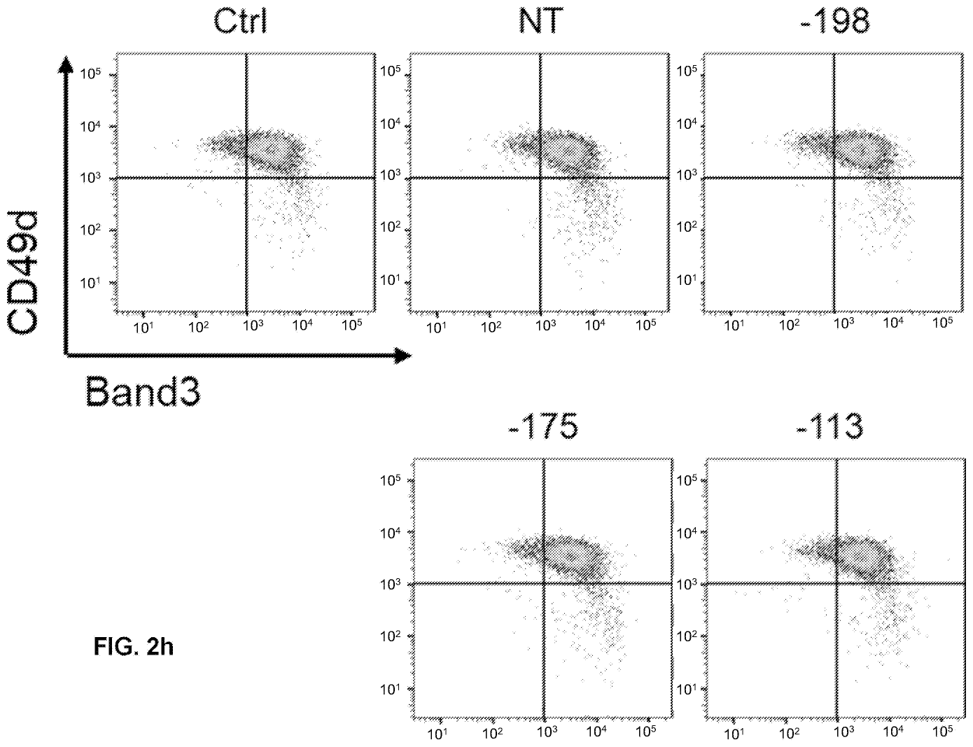

The inventors have found several advantages of the synthetic T→C mutation in the −175 proximal promoter region, which are illustrated in the appended figures. FIG. 1b-d compares the rates of base editing observed in the HUDEP-2 immortalized human erythroid progenitor cell line at each of these sites using an ABE7.10.0 adenosine base editor, while FIG. 2(a-c) compares rates of base editing at the same sites in healthy donor HSPCs. In certain embodiments, as illustrated in FIG. 1c and FIG. 2b, edits at the −175 site are characterized by a comparatively low rate of bystander editing (defined as edits to nearby non-targeted A:T base pairs), e.g., a rate of less than 20%, 15%, 10%, 5%, 4%, 3%, 2% or 1% of total edits. By contrast, rates of editing at the −198 site tended to be lower, and edits at both −198 and −113 sites are characterized by a greater frequency of bystander edits. Edits at the −175 site can also induce expression of fetal hemoglobin with comparatively greater efficiency than edits at either the −198 and −113 sites, as illustrated in FIG. 1(e-f) and 2(e-f). This efficiency is manifest as one or both of (a) an increase in the percentage of HSPC-derived cells expressing gamma globin and (b) by an increase in the fraction of fetal hemoglobin relative to total hemoglobin measured in cellular lysates. Additionally, bystander edits at the −175 site do not appear to negatively affect fetal hemoglobin expression in HUDEP-2 cells, while at least one bystander edit at the −113 site (referred to as G9 in FIG. 1a) may reduce the expression of fetal hemoglobin in cell lysates, as shown in FIG. 1f.

Various embodiments of this disclosure cause. induce or otherwise relate to synthetic mutations including those at the −175 site described above. Synthetic mutations of this disclosure include (but are not necessarily limited to) changes to genomic DNA sequences caused by the action of any suitable exogenous gene editing system, for instance a site-specific or programmed (e.g., an RNA-guided) endonuclease such as Cas9 or Cas12a, an engineered zinc-finger nuclease (ZFN) or transcription-activator-like effector nuclease (TALEN), as well as by base editors based on such endonucleases. Synthetic mutations are generated, in various embodiments, ex vivo e.g., by editing of HSPCs outside of the body, in which case the edited HSPCs, or cells derived therefrom, may be returned to the body of a subject after generation of the synthetic mutation. Alternatively. or additionally, synthetic mutations according to this disclosure may be caused by gene editing or base editing systems that are delivered to subjects in vivo, for instance as viral vectors (e.g., lentiviral or adeno-associated virus vectors) encoding one or more components of a base editing system of the present, lipid nanoparticles encapsulating DNA or RNA encoding one or more components of a base-editing system, or other suitable in vivo delivery methods.

Synthetic mutations according to this disclosure may be generated using adenosine deaminase base editors, such as those described above. Alternatively. or additionally. synthetic mutations may be introduced into cells by integration of a transgene into the native gene locus or into an alternate site such as a safe-harbor site. Without wishing to be bound by any theory. certain synthetic mutations according to this disclosure involve the creation of a new binding site for a transcriptional activator or other transcription factor more generally. For instance, the −175 T→C mutation disclosed herein can create a new consensus binding site for TAL1, which in turn may drive increased transcription of the HBG1 and HBG2 genes.

A base editor may form one. two or more synthetic mutations in the HBG1 and HBG2 upstream promoter regions (e.g., at the −175 position thereof). Accordingly, certain embodiments of this disclosure relate to a cell comprising at least one synthetic T to C mutation at a −175 proximal promoter site of HBG1 or HBG2. A cell of this disclosure may comprise a single allele (i.e., it may be heterozygous) of HBG1 or HGB2 bearing the synthetic mutation, it may be heterozygous for the synthetic allele at both HBG1 and HGB2, or it may be homozygous for the synthetic allele at one or both of HBG1 and HGB2. This disclosure also encompasses groups. populations or pluralities of cells, in which some (e.g., at least one, a plurality, a majority) bear one or more synthetic T→C mutations at a −175 proximal promoter site of HBG1 or HBG2 which are, optionally, made by one of the methods of this disclosure. The population of cells, in some cases, comprises primary cells taken from the body of a subject and edited ex vivo to introduce synthetic mutations. and/or cells expanded from and/or derived from such primary cells (i.e., ex vivo edited autologous cells). However, this disclosure also encompasses cell lines bearing one or more synthetic alleles, which are maintained in vitro and administered to subject who are different than the subject(s) from whom the cells or their progenitors were initially taken (i.e., allogeneic cells).

The populations of cells provided by embodiments of this disclosure may be substantially homogeneous—i.e., substantially all the same—or they may be heterogeneous—e.g., comprising first and second cells that differ from one another in genotype or phenotype. As non-limiting examples, populations of cells disclosed herein may be characterized by 50%, 60%, 70%, 80%, 90%, 95%, 99% or more of the cells comprising the same genotype or phenotype. For example, in some embodiments a population of cells comprises 50%, 60%, 70%, 80%, 90%, 95%, 99% or more cells (i) having at least one synthetic T→C mutation at a −175 proximal promoter site of an HBG1 or HBG2 gene; (ii) expressing fetal hemoglobin mRNA or protein; (iii) that are CD34+; (iv) having fewer than 1, 2, 3 or any other suitable threshold value of off-target edits; and/or (v) that stably engraft in a subject for 1, 2, 3, 4, 6, 8, 12, 18 or more months. Other art-known genotypic and phenotypic characteristics useful in defining cells and populations thereof may include viability, potency or differentiation potential, and the like.

Cells and populations of cells according to this disclosure may be characterized using methods established in the art, such as measurement of protein or mRNA expression by, e.g., flow cytometry, in situ hybridization, immunochemical methods such as ELISA, Sanger sequencing, next-generation sequencing (NGL). Additional suitable methods for characterization of cells and populations of cells will be evident to those of ordinary skill in the art.

This disclosure also encompasses methods of treating a subject, for instance to induce expression of fetal hemoglobin or to treat, prevent, palliate, ameliorate or otherwise affect a hemoglobinopathy such as sickle-cell disease. Methods of this disclosure can include contacting a subject with a cell or population of cells as described above. The contacting may be done by administering (e.g., by infusion, injection, transplantation, etc.) the cell or population of cells to the subject, or by contacting the subject with a base editing system or genome editing system that introduces a synthetic mutation into a cell or population of cells within the body of the subject. Where a cell or population of cells of this disclosure is administered to a subject, that cell or population can be, in various embodiments, autologous or allogeneic.

The exemplary embodiments described herein have, in some cases, utilized an ABE7.10 base editor to create the synthetic alleles of this disclosure. However, those of skill in the art will recognize that any suitable base editor can be used to introduce the synthetic alleles disclosed herein, including without limitation an ABE 8.0 base editor, which performs substantially similarly and the use of which is within the scope of this disclosure. More generally, the embodiments presented in this disclosure are intended to be illustrative rather than limiting, and those of skill in the art may readily identify modifications to the embodiments described herein that are known or evident to those of ordinary skill in the art; these modifications, too, are within the scope of the present disclosure.

### Methods

Human subjects research. Plerixafor-mobilized CD34+ cells from patients with SCD were collected according to the protocol Peripheral Blood Stem Cell Collection for Sickle Cell Disease Patients (www.clinicaltrials.gov identifier #NCT03226691), which was approved by the human subject research institutional review boards at the National Institutes of Health and St. Jude Children's Research Hospital. All patients provided informed consent.

Animal care. Mice were housed and handled in strict accordance with the recommendations in the Guide for the Care and Use of Laboratory Animals of the National Institutes of Health. Animal experiments were carried out in accordance with a protocol (Genetic Tools for the Study of Hematopoiesis) approved by the institutional animal care and use committee of the St. Jude Children's Research Hospital.

Isolation and maintenance of CD34+ hematopoietic stem and progenitor cells (HSPCs). Circulating G-CSF-mobilized human mononuclear cells were obtained from deidentified healthy adult donors (Key Biologics, Lifeblood). Plerixafor-mobilized mononuclear cells were isolated from SCD patients. Enrichment of CD34+ cells was performed by immunomagnetic bead selection using an AutoMACS instrument (Miltenyi Biotec, Gladbach, Germany). CD34+ cells were maintained in X-VIVO-10 (Lonza, Basel, Switzerland) media supplemented with SCF (stem cell factor, 100 ng/μL), TPO (thrombopoietin, 100 ng/μL) and Flt-3 ligand (100 ng/μL) with seeding density of 1×10⁶ cells/mL (see Table 1, below).

TABLE 1
cell culture, erythroid differentiation and reagents used

| Medium | Component | Manufacturer | Catalog # | Final Concentration | |
|---|---|---|---|---|---|
| CD34⁺ | X-vivo 10 (base) | Lonza | BP02-055Q | | |
| maintenance | Human Stem Cell | R&D systems | rhSCF, 255- | 100 | ng/mL |
| | Factor (hSCF) | | SC/CF | | |
| | Thrombopoietin | R&D systems | rhTPO # 288- | 100 | ng/mL |
| | (TPO) | | TP/CF | | |
| | FLT3-Ligand (FLT3- | R&D systems | rhFlt-3 # 3088- | 100 | ng/mL |
| | L) | | FK/CF) | | |
| | Penicillin- | Thermo Fisher | 15070063 | Penicillin: 50 U/mL | |
| | Streptomycin | Scientific | | Streptomycin: 50 | |
| | | | | μg/mL | |
| Erythroid | Common to all | | | | |
| differentiation | phases: | | | | |
| | IMDM (base) | Thermo Fisher | 12440061 | | |
| | | Scientific | | | |
| | Human Male AB | SeraCare | 1810-0001 | 2% | |
| | Plasma | | | | |
| | Human AB Serum | Atlanta Biologicals | S40110 | 3% | |
| | Heparin | Sagent | NDC # 25021- | 3 | UI/mL |
| | | Pharmaceuticals | 401-02 | | |
| | Insulin | Lilly | Humulin ® R U- | 10 | μg/mL |
| | | | 100, NDC # 0002- | | |
| | | | 8215-17 | | |
| | EPO | Amgen | EPOGEN ®, NDC | 3 | UI/mL |
| | | | # 55513-144-01 | | |
| | Penicillin- | Thermo Fisher | 15070063 | Penicillin 50 U/mL | |
| | Streptomycin | Scientific | | Streptomycin 50 | |
| | | | | μg/mL | |
| Erythroid | Add the following to | | | | |
| differentiation | common components: | | | | |
| (phase I) | | | | | |
| | Human Holo- | Millipore Sigma | T0665 | 200 | μg/mL |
| | Transferrin | | | | |
| | Human Stem Cell | R&D systems | rhSCF, 255- | 10 | ng/mL |
| | Factor (hSCF) | | SC/CF | | |
| | Human IL-3 | R&D systems | rhIL-3, 203-IL/CF | 1 | ng/mL |
| Erythroid | Add the following to | | | | |
| Differentiation | common components: | | | | |
| (phase II) | | | | | |
| | Human Holo- | Millipore Sigma | T0665 | 200 | μg/mL |
| | Transferrin | | | | |
| | Human Stem Cell | R&D systems | rhSCF, 255- | 10 | ng/mL |
| | Factor (hSCF) | | SC/CF | | |
| Erythroid | Add the following to | | | | |
| differentiation | common components: | | | | |
| (phase III) | | | | | |
| | Human Holo- | Millipore Sigma | T0665 | 1 | mg/mL |
| | Transferrin | | | | |

Electroporation of ABE7.10 mRNA in HUDEP-2 cells. HUDEP-2 cells were electroporated with 2 μg of ABEmax mRNA (TriLink) and 2 μg of sgRNA (Sythego Corporation). AMAXA Cell Line Nucleofector Kit (Lonza, Basel, Switzerland, catalog #V4SP-3096) was used with DS-150 program (Nucleofector II).

RNP electroporation in HSPCs. Electroporation was performed using Lonza 4D Nucleofector (V4SP-3096 for 20-μl Nucleocuvette Strips or V4SP-3096 for 20-μl Nucleocuvette Strips) according to the manufacturer's instructions. The modified synthetic sgRNA (2′-O-methyl 3′ phosphorothioate modifications in the first and last three nucleotides) was from Synthego (Redwood City, CA) (see Table 2, below). CD34+ HSPCs were thawed 24 hours before electroporation. ABE7.10 and ABE7.10max-SpCas9-NG protein was ordered as a custom product from Aldevron and delivered at a stock concentration of 94 μM and 45.9 μM respectively. For 20-μl Nucleocuvette Strips, the RNP complex was prepared by mixing ABE7.10 protein (100 pmol) (Aldevron) and sgRNA (300 pmol) and incubating for 15 min (or 20 min) at room temperature immediately before electroporation. Two hundred thousand to 2 million HSPCs resuspended in 20 μl of P3 solution were mixed with RNP and transferred to a cuvette for electroporation with program DS-130. Cas9 nuclease (3xNLS) and sgRNA targeting HBG1/2 promoter were electroporated as described previously. For 100-μl cuvette electroporation, the RNP complex was made by mixing 500 pmol ABE7.10 or ABE7.10max-SpCas9 protein and 1500 pmol sgRNA. Next, 2.5-5 million HSPCs were resuspended in 100 μl of P3 solution for RNP electroporation as described above. Electroporated cells were recovered in X-VIVO 10 media including cytokines. Genomic DNA was extracted on culture day 3 (or on culture days 3, 6, and 9) by using Qiagen buffer then analyzed by NGS for editing efficiency.

TABLE 2
sequences used

| Assay | Name | Sequence (5′ to 3′) |
|---|---|---|
| Single guide | | Underlined sequence corresponds to target |
| RNA | sgRNA-198 | G*G*U*GGGGAAGGGGCCCCCAAGGUUUUAGAGCUAGAAAUAG |
| | (SEQ ID | CAAGUUAAAAUAAGGCUAGUCCGUUAUCAACUUGAAAAAGUG |
| | NO: 1) | GCACCGAGUCGGUGCU*U*U*U |
| | sgRNA-175 | G*A*G*AUAUUUGCAUUGAGAUAGGUUUUAGAGCUAGAAAUAG |
| | (SEQ ID | CAAGUUAAAAUAAGGCUAGUCCGUUAUCAACUUGAAAAAGUG |
| | NO: 2) | GCACCGAGUCGGUGCU*U*U*U |
| | sgRNA-113 | G*C*U*UGACCAAUAGCCUUGACAGUUUUAGAGCUAGAAAUAG |
| | (SEQ ID | CAAGUUAAAAUAAGGCUAGUCCGUUAUCAACUUGAAAAAGUG |
| | NO: 3) | GCACCGAGUCGGUGCU*U*U*U |
| | | *2′-O-methyl modification and phosphorothioate |
| | | inter-nucleotide linkage. Chemically modified |
| | | versions of sgRNAS contained 2′-O-methyl |
| | | modifications and phosphorothioate linkages |
| | | in the first and last 3 nucleotides. gRNAs |
| | | synthesized by Synthego contain an additional |
| | | 5′ G. |
| Base Editor | ABE-max | ATGAAACGGACAGCCGACGGAAGCGAGTTCGAGTCACCAAAGA |
| Expression | NG SpCas9 | AGAAGCGGAAAGTCTCTGAAGTCGAGTTTAGCCACGAGTATTGG |
| Plasmid | plasmid ORF | ATGAGGCACGCACTGACCCTGGCAAAGCGAGCATGGGATGAAA |
| | (SEQ ID | GAGAAGTCCCCGTGGGCGCCGTGCTGGTGCACAACAATAGAGTG |
| | NO: 4) | ATCGGAGAGGGATGGAACAGGCCAATCGGCCGCCACGACCCTA |
| | | CCGCACACGCAGAGATCATGGCACTGAGGCAGGGAGGCCTGGT |
| | | CATGCAGAATTACCGCCTGATCGATGCCACCCTGTATGTGACAC |
| | | TGGAGCCATGCGTGATGTGCGCAGGAGCAATGATCCACAGCAG |
| | | GATCGGAAGAGTGGTGTTCGGAGCACGGGACGCCAAGACCGGC |
| | | GCAGCAGGCTCCCTGATGGATGTGCTGCACCACCCCGGCATGAA |
| | | CCACCGGGTGGAGATCACAGAGGGAATCCTGGCAGACGAGTGC |
| | | GCCGCCCTGCTGAGCGATTTCTTTAGAATGCGGAGACAGGAGAT |
| | | CAAGGCCCAGAAGAAGGCACAGAGCTCCACCGACTCTGGAGGA |
| | | TCTAGCGGAGGATCCTCTGGAAGCGAGACACCAGGCACAAGCG |
| | | AGTCCGCCACACCAGAGAGCTCCGGCGGCTCCTCCGGAGGATCC |
| | | TCTGAGGTGGAGTTTTCCCACGAGTACTGGATGAGACATGCCCT |
| | | GACCCTGGCCAAGAGGGCACGCGATGAGAGGGAGGTGCCTGTG |
| | | GGAGCCGTGCTGGTGCTGAACAATAGAGTGATCGGCGAGGGCT |
| | | GGAACAGAGCCATCGGCCTGCACGACCCAACAGCCCATGCCGA |
| | | AATTATGGCCCTGAGACAGGGCGGCCTGGTCATGCAGAACTACA |
| | | GACTGATTGACGCCACCCTGTACGTGACATTCGAGCCTTGCGTG |
| | | ATGTGCGCCGGCGCCATGATCCACTCTAGGATCGGCCGCGTGGT |
| | | GTTTGGCGTGAGGAACGCAAAAACCGGCGCCGCAGGCTCCCTGA |
| | | TGGACGTGCTGCACTACCCCGGCATGAATCACCGCGTCGAAATT |
| | | ACCGAGGGAATCCTGGCAGATGAATGTGCCGCCCTGCTGTGCTA |
| | | TTTCTTTCGGATGCCTAGACAGGTGTTCAATGCTCAGAAGAAGG |
| | | CCCAGAGCTCCACCGACTCCGGAGGATCTAGCGGAGGCTCCTCT |
| | | GGCTCTGAGACACCTGGCACAAGCGAGAGCGCAACACCTGAAA |
| | | GCAGCGGGGGCAGCAGCGGGGGGTCAGACAAGAAGTACAGCAT |
| | | CGGCCTGGCCATCGGCACCAACTCTGTGGGCTGGGCCGTGATCA |
| | | CCGACGAGTACAAGGTGCCCAGCAAGAAATTCAAGGTGCTGGG |
| | | CAACACCGACCGGCACAGCATCAAGAAGAACCTGATCGGAGCC |
| | | CTGCTGTTCGACAGCGGCGAAACAGCCGAGGCCACCCGGCTGAA |
| | | GAGAACCGCCAGAAGAAGATACACCAGACGGAAGAACCGGATC |
| | | TGCTATCTGCAAGAGATCTTCAGCAACGAGATGGCCAAGGTGGA |
| | | CGACAGCTTCTTCCACAGACTGGAAGAGTCCTTCCTGGTGGAAG |
| | | AGGATAAGAAGCACGAGCGGCACCCCATCTTCGGCAACATCGTG |
| | | GACGAGGTGGCCTACCACGAGAAGTACCCCACCATCTACCACCT |
| | | GAGAAAGAAACTGGTGGACAGCACCGACAAGGCCGACCTGCGG |
| | | CTGATCTATCTGGCCCTGGCCCACATGATCAAGTTCCGGGGCCA |
| | | CTTCCTGATCGAGGGCGACCTGAACCCCGACAACAGCGACGTGG |
| | | ACAAGCTGTTCATCCAGCTGGTGCAGACCTACAACCAGCTGTTC |
| | | GAGGAAAACCCCATCAACGCCAGCGGCGTGGACGCCAAGGCCA |
| | | TCCTGTCTGCCAGACTGAGCAAGAGCAGACGGCTGGAAAATCTG |
| | | ATCGCCCAGCTGCCCGGCGAGAAGAAGAATGGCCTGTTCGGAA |
| | | ACCTGATTGCCCTGAGCCTGGGCCTGACCCCCAACTTCAAGAGC |
| | | AACTTCGACCTGGCCGAGGATGCCAAACTGCAGCTGAGCAAGG |
| | | ACACCTACGACGACGACCTGGACAACCTGCTGGCCCAGATCGGC |
| | | GACCAGTACGCCGACCTGTTTCTGGCCGCCAAGAACCTGTCCGA |
| | | CGCCATCCTGCTGAGCGACATCCTGAGAGTGAACACCGAGATCA |
| | | CCAAGGCCCCCCTGAGCGCCTCTATGATCAAGAGATACGACGAG |
| | | CACCACCAGGACCTGACCCTGCTGAAAGCTCTCGTGCGGCAGCA |
| | | GCTGCCTGAGAAGTACAAAGAGATTTTCTTCGACCAGAGCAAGA |
| | | ACGGCTACGCCGGCTACATTGACGGCGGAGCCAGCCAGGAAGA |
| | | GTTCTACAAGTTCATCAAGCCCATCCTGGAAAAGATGGACGGCA |
| | | CCGAGGAACTGCTCGTGAAGCTGAACAGAGAGGACCTGCTGCG |
| | | GAAGCAGCGGACCTTCGACAACGGCAGCATCCCCCACCAGATCC |
| | | ACCTGGGAGAGCTGCACGCCATTCTGCGGCGGCAGGAAGATTTT |
| | | TACCCATTCCTGAAGGACAACCGGGAAAAGATCGAGAAGATCCT |
| | | GACCTTCCGCATCCCCTACTACGTGGGCCCTCTGGCCAGGGGAA |
| | | ACAGCAGATTCGCCTGGATGACCAGAAAGAGCGAGGAAACCAT |
| | | CACCCCCTGGAACTTCGAGGAAGTGGTGGACAAGGGCGCTTCCG |
| | | CCCAGAGCTTCATCGAGCGGATGACCAACTTCGATAAGAACCTG |
| | | CCCAACGAGAAGGTGCTGCCCAAGCACAGCCTGCTGTACGAGTA |
| | | CTTCACCGTGTATAACGAGCTGACCAAAGTGAAATACGTGACCG |
| | | AGGGAATGAGAAAGCCCGCCTTCCTGAGCGGCGAGCAGAAAAA |
| | | GGCCATCGTGGACCTGCTGTTCAAGACCAACCGGAAAGTGACCG |
| | | TGAAGCAGCTGAAAGAGGACTACTTCAAGAAAATCGAGTGCTTC |
| | | GACTCCGTGGAAATCTCCGGCGTGGAAGATCGGTTCAACGCCTC |
| | | CCTGGGCACATACCACGATCTGCTGAAAATTATCAAGGACAAGG |
| | | ACTTCCTGGACAATGAGGAAAACGAGGACATTCTGGAAGATATC |
| | | GTGCTGACCCTGACACTGTTTGAGGACAGAGAGATGATCGAGGA |
| | | ACGGCTGAAAACCTATGCCCACCTGTTCGACGACAAAGTGATGA |
| | | AGCAGCTGAAGCGGCGGAGATACACCGGCTGGGGCAGGCTGAG |
| | | CCGGAAGCTGATCAACGGCATCCGGGACAAGCAGTCCGGCAAG |
| | | ACAATCCTGGATTTCCTGAAGTCCGACGGCTTCGCCAACAGAAA |
| | | CTTCATGCAGCTGATCCACGACGACAGCCTGACCTTTAAAGAGG |
| | | ACATCCAGAAAGCCCAGGTGTCCGGCCAGGGCGATAGCCTGCAC |
| | | GAGCACATTGCCAATCTGGCCGGCAGCCCCGCCATTAAGAAGGG |
| | | CATCCTGCAGACAGTGAAGGTGGTGGACGAGCTCGTGAAAGTG |
| | | ATGGGCCGGCACAAGCCCGAGAACATCGTGATCGAAATGGCCA |
| | | GAGAGAACCAGACCACCCAGAAGGGACAGAAGAACAGCCGCGA |
| | | GAGAATGAAGCGGATCGAAGAGGGCATCAAAGAGCTGGGCAGC |
| | | CAGATCCTGAAAGAACACCCCGTGGAAAACACCCAGCTGCAGA |
| | | ACGAGAAGCTGTACCTGTACTACCTGCAGAATGGGCGGGATATG |
| | | TACGTGGACCAGGAACTGGACATCAACCGGCTGTCCGACTACGA |
| | | TGTGGACCATATCGTGCCTCAGAGCTTTCTGAAGGACGACTCCA |
| | | TCGACAACAAGGTGCTGACCAGAAGCGACAAGAACCGGGGCAA |
| | | GAGCGACAACGTGCCCTCCGAAGAGGTCGTGAAGAAGATGAAG |
| | | AACTACTGGCGGCAGCTGCTGAACGCCAAGCTGATTACCCAGAG |
| | | AAAGTTCGACAATCTGACCAAGGCCGAGAGAGGCGGCCTGAGC |
| | | GAACTGGATAAGGCCGGCTTCATCAAGAGACAGCTGGTGGAAA |
| | | CCCGGCAGATCACAAAGCACGTGGCACAGATCCTGGACTCCCGG |
| | | ATGAACACTAAGTACGACGAGAATGACAAGCTGATCCGGGAAG |
| | | TGAAAGTGATCACCCTGAAGTCCAAGCTGGTGTCCGATTTCCGG |
| | | AAGGATTTCCAGTTTTACAAAGTGCGCGAGATCAACAACTACCA |
| | | CCACGCCCACGACGCCTACCTGAACGCCGTCGTGGGAACCGCCC |
| | | TGATCAAAAAGTACCCTAAGCTGGAAAGCGAGTTCGTGTACGGC |
| | | GACTACAAGGTGTACGACGTGCGGAAGATGATCGCCAAGAGCG |
| | | AGCAGGAAATCGGCAAGGCTACCGCCAAGTACTTCTTCTACAGC |
| | | AACATCATGAACTTTTTCAAGACCGAGATTACCCTGGCCAACGG |
| | | CGAGATCCGGAAGCGGCCTCTGATCGAGACAAACGGCGAAACC |
| | | GGGGAGATCGTGTGGGATAAGGGCCGGGATTTTGCCACCGTGCG |
| | | GAAAGTGCTGAGCATGCCCCAAGTGAATATCGTGAAAAAGACC |
| | | GAGGTGCAGACAGGCGGCTTCAGCAAAGAGTCTATCCGGCCCA |
| | | AGAGGAACAGCGATAAGCTGATCGCCAGAAAGAAGGACTGGGA |
| | | CCCTAAGAAGTACGGCGGCTTCGTGAGCCCCACCGTGGCCTATT |
| | | CTGTGCTGGTGGTGGCCAAAGTGGAAAAGGGCAAGTCCAAGAA |
| | | ACTGAAGAGTGTGAAAGAGCTGCTGGGGATCACCATCATGGAA |
| | | AGAAGCAGCTTCGAGAAGAATCCCATCGACTTTCTGGAAGCCAA |
| | | GGGCTACAAAGAAGTGAAAAAGGACCTGATCATCAAGCTGCCT |
| | | AAGTACTCCCTGTTCGAGCTGGAAAACGGCCGGAAGAGAATGCT |
| | | GGCCTCTGCCCGGTTCCTGCAGAAGGGAAACGAACTGGCCCTGC |
| | | CCTCCAAATATGTGAACTTCCTGTACCTGGCCAGCCACTATGAG |
| | | AAGCTGAAGGGCTCCCCCGAGGATAATGAGCAGAAACAGCTGT |
| | | TTGTGGAACAGCACAAGCACTACCTGGACGAGATCATCGAGCAG |
| | | ATCAGCGAGTTCTCCAAGAGAGTGATCCTGGCCGACGCTAATCT |
| | | GGACAAAGTGCTGTCCGCCTACAACAAGCACCGGGATAAGCCC |
| | | ATCAGAGAGCAGGCCGAGAATATCATCCACCTGTTTACCCTGAC |
| | | CAATCTGGGAGCCCCTCGGGCCTTCAAGTACTTTGACACCACCA |
| | | TCGACCGGAAGGTGTACCGGAGCACCAAAGAGGTGCTGGACGC |
| | | CACCCTGATCCACCAGAGCATCACCGGCCTGTACGAGACACGGA |
| | | TCGACCTGTCTCAGCTGGGAGGTGACTCTGGCGGCTCAAAAAGA |
| | | ACCGCCGACGGCAGCGAATTCGAGCCCAAGAAGAAGAGGAAAG |
| | | TCTAA |

Measurements of on-target base editing and indel frequencies, NGS library preparation and analysis. Targeted amplicons were generated using gene specific primers with partial Illumina adapter overhangs (hHBG.F-5′-TGACTGAATCGGAACAAGGCAAAGG-3′ (SEQ ID NO: 5) and hHBG.R-5′-ATTCTTCATCCCTAGCCAGCCGC-3′ (SEQ ID NO: 6), overhangs not shown) and sequenced as previously described. Briefly, cell pellets of approximately 0.1 million cells were lysed and used to generate gene specific amplicons with partial Illumina adapters in PCR #1. Amplicons were indexed in PCR #2 and pooled with other targeted amplicons for other loci to create sequence diversity. Additionally, 10% PhiX Sequencing Control V3 (Illumina) was added to the pooled amplicon library prior to running the sample on an Miseq Sequencer System (Illumina) to generate paired 2× 250 bp reads. Samples were demultiplexed using the index sequences, fastq files were generated, and NGS analysis was performed using CRIS.py.

Erythroid cell culture. Erythroid differentiation of CD34+ cells was performed using a 3-phase protocol as described previously (see Table 1). Phase 1 (days 1-7): IMDM with 2% human blood type AB plasma, 3% human AB serum, 1% penicillin/streptomycin, 3 units/mL heparin, 3 units/mL EPO, 200 μg/mL holo-transferrin, 10 ng/mL SCF, and 1 ng/mL interleukin IL-3. Phase 2 (days 8-14): Phase 1 medium without IL-3. Phase 3 (days 15-21): The holo-transferrin concentration was increased to 1 mg/mL, and SCF was withdrawn.

Erythroblast maturation was monitored by immuno-flow cytometry for the cell surface markers CD235a, CD49d, and Band3 (see Table 3). To quantify erythroblast enucleation and f-cell analysis, 1.5-5×10⁶ CD34+-derived erythroid cells were incubated with Hoechst 33342 for 20 min at 37° C., fixed with 0.05% glutaraldehyde (Millipore Sigma, G5882), and permeabilized with 0.1% Triton X-100 (Millipore Sigma, 93443). Cells were stained with FITC mouse anti-human CD235a and anti-human HbF-APC then analyzed by flow cytometry.

Clonal culture of CD34+ HSPCs. Base edited (sgRNA −175) CD34+ HSPCs were sorted into 100 μl phase-1 erythroid differentiation media in 96-well round-bottom plates (Corning) at one cell per well using FACSAria III. The cells were changed into phase-2 erythroid differentiation media 7 days later in 96-well flat-bottom plates (Corning). After 14 days cells in each well were collected for genotyping analysis and hemoglobin HPLC measurement per colony.

HUDEP Cell culture. HUDEP clone 2 (HUDEP-2) cells were cultured as previously described elsewhere. Cells were expanded in StemSpan SFEM (Stem Cell Technologies) supplemented with 1 μM dexamethasone, 1 μg/mL doxycycline. 50 ng/ml human SCF, 3 units/mL EPO, and 1% penicillin/streptomycin. HUDEP-2 cells were differentiated in the Phase III medium used for CD34+ erythroid cultures using a 2-phase protocol. Phase 1 (days 1-7): IMDM with 2% fetal bovine serum, 2% human blood type AB plasma, 1% penicillin/streptomycin, 3 units/mL heparin, 10 μg/mL insulin, 3 units/mL EPO, 1 mg/mL holo-transferrin, 50 ng/mL SCF and 1 μg/mL doxycycline. Phase 2 (days 7-10) phase 1 media without SCF.

Clonal culture of HUDEP-2 cells. HUDEP-2 cells were electroporated with RNP complex comprising of Cas9 nuclease (2xNLS) and sgRNAs paired (sgRNA-1: ACCCGGAACTCCCTCAAGCA (SEQ ID NO: 7); sgRNA-2: ATTTCCATTAGATTCATTAG (SEQ ID NO: 8)) located upstream of HBE and downstream of HBB genes. Edited HUDEP-2 cells were sorted into 100-μl expansion media in 96-well round-bottom plates (Corning) at one cell per well using SH800 sorter (Sony Biotechnologies). HUDEP-2 clones carrying one copy of beta-like globin genes (hemizygous) were picked for further study (FIG. 1i). The hemizygous HUDEP-2 cells were electroporated with ABE7.10 or ABE8e mRNA with sgRNAs targeting HPFH mutations and GATA1 motif at HBG1/2 gene promoter (see Table 2). Edited hemizygous HUDEP-2 cells were sorted as mentioned above. The cells were changed to 24-well plates (Corning) 14 days later. After an additional 4 days of culture, the cells were changed into 3 ml Phase III media for differentiation. After an additional 7 days of culture, half of the cells were harvested for genotyping analysis and half for a single hemoglobin HPLC measurement per colony.

HbF quantification. Analytical high performance liquid chromatography (HPLC) quantification of hemoglobin tetramers and individual globin chains was performed using ion-exchange and reverse-phase columns on a Prominence HPLC System (Shimadzu Corporation). Proteins eluted from the column were identified at 220 and 415 nm with a diode array detector. The relative amounts of hemoglobins or individual globin chains were calculated from the area under the 415-nm peak and normalized based on the dimethyl sulfoxide (DMSO) control. The percentage HbF was calculated as follows, % HbF=[HbF/(HbA+HbF)]×100; % gamma-globin=[(G-gamma-chain+A-gamma-chain)/beta-like chains (beta+G-gamma+A-gamma)]×100.

In vitro sickling assay. CD34+ HSPCs derived erythroid cells on day 21 or MACS purified CD235a+ cells from bone marrow were incubated with Hoechst 33342 for 20 min at 37° C., Hoechst negative population were sorted using a SH1800 sorter (Sony Biotechnologies). Sorted cells (0.5-1.0×10⁵ cells) were recovered for 24h or 3-4 days in phase 3 ED medium and seeded in 12 well or Poly-L-lysine treated 96 well plate with 0.1 mL or 1 mL of phase 3 media ED medium under hypoxic conditions (2% O2) for 24 h. The IncuCyte® S3 Live-Cell Analysis System (Sartorius) with a 20× objective was used to monitor cell sickling, with images being captured every 20-30 minutes. The percentage of sickling was measured at 8 h in hypoxia by manual counting of sickled cells versus normal cells based on morphology.

Xenotransplantation of gene-edited CD34+ HSPCs into NOD.Cg-KitW-41J Tyr+ Prkdescid Il2rgtm1Wjl/ThomJ (NBSGW) mice. NBSGW mice were purchased from The Jackson Laboratory (stock no. 026622). Base edited or control CD34+ cells were administered at a dose of 0.2-0.5×10⁶ per mouse by tail-vein injection in female mice aged 8-12 weeks. Chimerism post-transplantation was evaluated at 16-18 weeks in the bone marrow at the time of euthanasia (see Table 3). Cell lineage composition was determined in the bone marrow by using human-specific antibodies (see Table 3), and lineages were sorted using a FACSAria III cell sorter (BD Biosciences). CD34+ HSPCs or CD235a+ erythroblasts were isolated with magnetic beads, using the human-specific CD34 MicroBead Kit UltraPure, human, (Miltenyi Biotec Inc., catalog #130-100-453) and CD235a (glycophorin A) MicroBeads, human, (Miltenyi Biotec Inc., catalog #130-050-501)].

TABLE 3
Antibodies used in flow cytometry panels

| Panel | Antibody | Clone | Manufacturer | Catalog # |
|---|---|---|---|---|
| Erythropoiesis and | FITC Mouse Anti-Human | GA-R2 (HIR2) | BD | 559943 |
| fetal hemoglobin | CD235a | (RUO) | Pharmingen ™ | |
| expression | Hoechst 33342 | Stock 10 mM = | Millipore Sigma | B2261 |
| | | 2000× | | |
| | PE Anti-Human CD49d | 9F10 | BioLegend | 304304 |
| | APC Anti-Human Band3 | | New York Blood | Gift from X. |
| | | | Center | An |
| | Anti-Fetal Hemoglobin-APC, | REA533 | Miltenyi Biotec | 130-108-243 |
| | human | | | |
| Chimerism after | FITC Rat Anti-Mouse CD45 | 30-F11 (RUO) | BD Horizon ™ | 561088 |
| xenotransplantation | PerCP-Cy ™5.5 Rat Anti- | TER-119 (RUO) | BD | 560512 |
| | Mouse TER-119/Erythroid | | Pharmingen ™ | |
| | Cells | | | |
| (Bone marrow) | BV605 Mouse Anti-Human | HI30 (RUO) | BD Horizon ™ | 564047 |
| | CD45 | | | |
| | PE-Cy ™7 Mouse Anti- | P67.6 (RUO | BD Biosciences | 333946 |
| | Human CD33 | (GMP)) | | |
| | APC-Cy ™7 Mouse Anti- | SK7 (Leu-4) | BD | 557832 |
| | Human CD3 | (RUO) | Pharmingen ™ | |
| | CD19 (Leu ™-12) PE | 4G7 (IVD) | BD Biosciences | 349209 |
| | Alexa, Flour, 700 Mouse | 581 (RUO) | BD Biosciences | 561440 |
| | Anti-Human CD34 | | | |
| | APC Mouse Anti-Human | GA-R2 (HIR2) | BD | 551336 |
| | CD235a | (RUO) | Pharmingen ™ | |
| | DAPI | | | |

Reverse transcription digital droplet PCR. The concentration of the extracted RNA was measured using Nanodrop (Thermo Scientific). One-step reverse transcription digital droplet PCR (RT-ddPCR) was used to determine p21 mRNA expression change. Ribonuclease P/MRP subunit p30 (RPP30) was used as internal control. 3 ng of RNA was mixed with Reverse Transcriptase, 300 mM DTT, and Supermix in One-Step RT-ddPCR advanced kit for Probes (Bio-Rad, Hercules, CA. USA), p21 primers/probe (Bio-Rad, 10031252; Assay ID: dHsaCPE5052298), and RPP30 primers/probe (Bio-Rad, 10031255; Assay ID: dHsaCPE5038241) according to the manufacturer's protocol. After making droplets using Automated Droplet Generator (Bio-Rad), PCR was performed as follows: first step at 50° C. for 60 min, second step at 95° C. for 10 min, third step at 95° C. for 30 sec and then at 55° C. for 1 min (40 cycles), and fourth step at 98° C. for 10 min. Droplets were read using QX200™ (Bio-Rad) and data were analyzed using QuantaSoft (Bio-Rad).

qPCR detection of 4.9-kb deletion alleles. A primer and probe set were designed to detect amplification of a HBG1 promoter-specific sequence. TaqMan qPCR was performed on genomic DNA samples from CD34+ cells using Universal TaqMan Mix (Thermo Fisher Scientific) for quantification of triplicates for each sample. AACt values were calculated based on amplification of RNaseP (Thermo Fisher Scientific) for copy number reference.

4.9 kb Fwd:
(SEQ ID NO: 9)
ACGGATAAGTAGATATTGAGGTAAGC
4.9 kb Rev:
(SEQ ID NO: 10)
GTCTCTTTCAGTTAGCAGTGG
Taqman probe (FAM):
(SEQ ID NO: 11)
ACTGCGCTGAAACTGTGGTCTTTATGA

CUT&RUN. The CUT&RUN assay was performed to identify transcription factor occupancy profiles, as described previously. Approximately 0.5 million cells were analyzed for each reaction. The antibodies used are described in Table 2. DNA libraries were prepared using the NEB Next Ultra II DNA Library Prep Kit from NEB (E7645S). Indexed samples were analyzed by paired end NGS using the Illumina Next-seq 150-cycle kit. Data analysis and footprint processing were performed as described previously.

### EXAMPLES

This written description uses examples to disclose the disclosure, including the best mode, and also to enable any person skilled in the art to practice the disclosure, including making and using any devices or systems and performing any incorporated methods. The patentable scope of the disclosure is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

Any non-limiting examples are provided to further illustrate the present disclosure. It should be appreciated by those of skill in the art that the techniques disclosed in the examples represent approaches the inventors have found function well in the practice of the present disclosure, and thus can be considered to constitute examples of modes for its practice. However, those of skill in the art should, in light of the present disclosure. appreciate that many changes can be made in the specific embodiments that are disclosed and still obtain a like or similar result without departing from the spirit and scope of the present disclosure.

Certain principles of this disclosure are illustrated by the following examples. which are intended to be exemplary rather than limiting in nature.

### Example 1

### A Base Editor (ABE7.10) Recreates HPFH Mutations and Induces HbF in HUDEP-2 Cells.

Single guide RNAs (gRNAs) were designed targeting the HPFH mutations (−198 T→C, −175 T→C and −113 A→G) in gamma-globin gene promoter (FIG. 1a; see Table 2). mRNA delivery of individual sgRNAs and a base editor mRNA (ABEmax) into erythroid progenitor cell line (HUDEP-2) revealed a different on-target and by-stander editing efficiency for the sgRNAs targeting all 3 sites. First, for −198 site the editing frequency of target mutation ( −198 T→C; position C7) was approximately 41% with by-stander mutation ( −198 T→CC; position C8) <5% (FIG. 1. b). Second, for −175 site the editing frequency of target mutation ( −175 T→C; position C5) was approximately 73% with by-stander mutation (−181 T→C; position C11) ≤1% (FIG. 1.c). Third, at the −113 site the editing frequency of the target mutation at position 8 of the protospacer was approximately 59% with bystander mutations at positions A5 and A9 occurring with 64% and 10% efficiency, respectively (FIG. 1d). Further, low rates of indels were observed following editing: 3% at the −198 site and ≤1% at both the −175 and −113 sites (FIG. 1.g). The base edited HUDEP-2 cells were differentiated into mature erythroblasts to assess HbF induction following each of the three separate treatments. Flow cytometric analysis of cells edited at −198, −175, and −113 sites revealed a high frequency of HbF-expressing cells (F cells)—74±8%, 93±2%, and 93±1%, respectively. compared to 5+1% in non-edited cells (FIG. 1h). Analytical ion-exchange HPLC (IE-HPLC) was performed on lysates of mature erythrocytes to assess the fraction of HbF protein out of total beta-globin in these cells. In cellular populations edited at the −198, −175, and −113 sites, HbF content was measured at 18±4%, 52±6%, 33±0%, respectively. compared to 2±1% in non-edited cells (FIG. 1.e). The results suggest that −175 T→C site, which creates a binding site for TAL1, is more an even more potent for promising for HbF induction compared to other 2 sites. Next, hemizygous HUDEP-2 cells (harboring only a single copy of the HBG2 and HBG1 gene opposite a deletion of the region on the sister chromosome) (FIG. 1.i) were subjected to single-cell clonal erythroid liquid culture after base editing to assess the HbF induction resulting from editing outcomes. Clones with HPFH mutations at both the gamma-globin genes (HBG2 and HBG1) were picked for HPLC analysis. HbF content expression was increased to 51±19% on account for the −198 T→C edit, 84+9% on account for the −175 T→C edit. 30±20% on account for the −113 edit alone, 70±8% on account for the −116 edit alone, and 73±13% in cells with edits at both the −113 and −116 positions (FIG. 1.f). Clones with all three of the −112, −113, and −116 A→G edits were measured had decreased to have an HbF content expression of 38±9% (FIG. 1.f). Overall, these results that suggest that recreation of HPFH mutations in the gamma-globin proximal promoter induced HbF in erythroid cells. The −175 T→C mutation, which creates a de novo binding site for an erythroid transcription factor TAL1 showed robust induction of HbF compared to other HPFH mutations. Interestingly, the −116 A→G polymorphism that disrupts a BCL11A binding site led to even greater HbF induction relative to −113 A→G that creates a GATA1 binding site, while the combination of both edits yielded a synergistic effect on gamma-globin expression. The addition of a third −112 A→G edit decreased the levels of HbF induction, perhaps because it disrupts due to the disruption of the new GATA1 binding motif created by the −113 edit.

ABE7.10 Recreates HPFH Mutations in Normal Donor CD34+ Cells and Induces HbF in their Erythroid Progeny

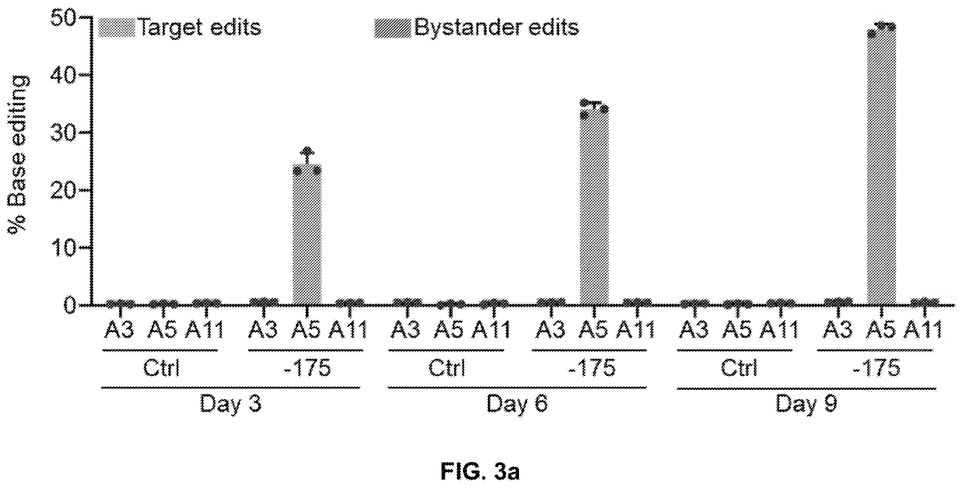

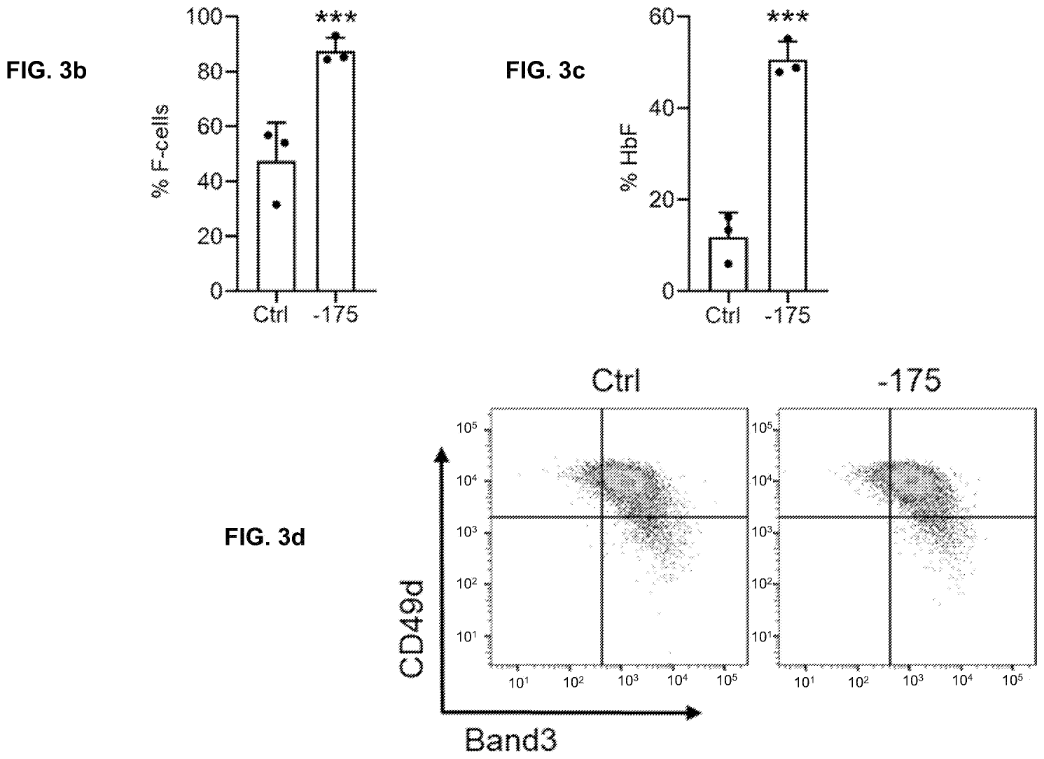

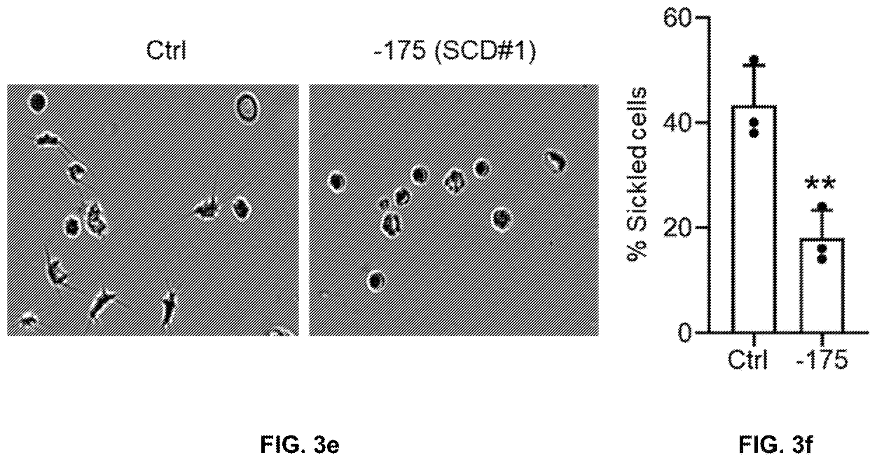

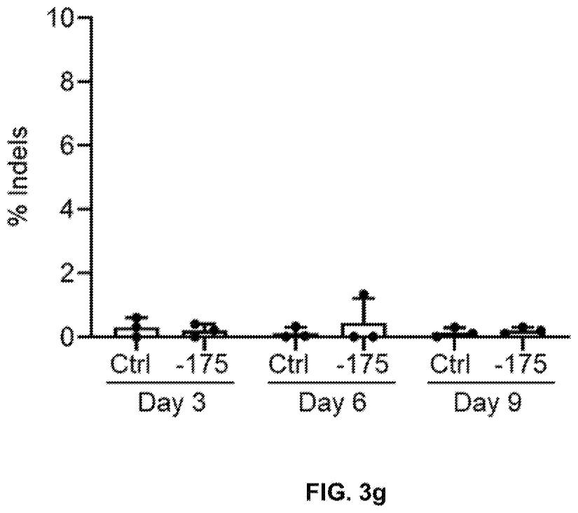

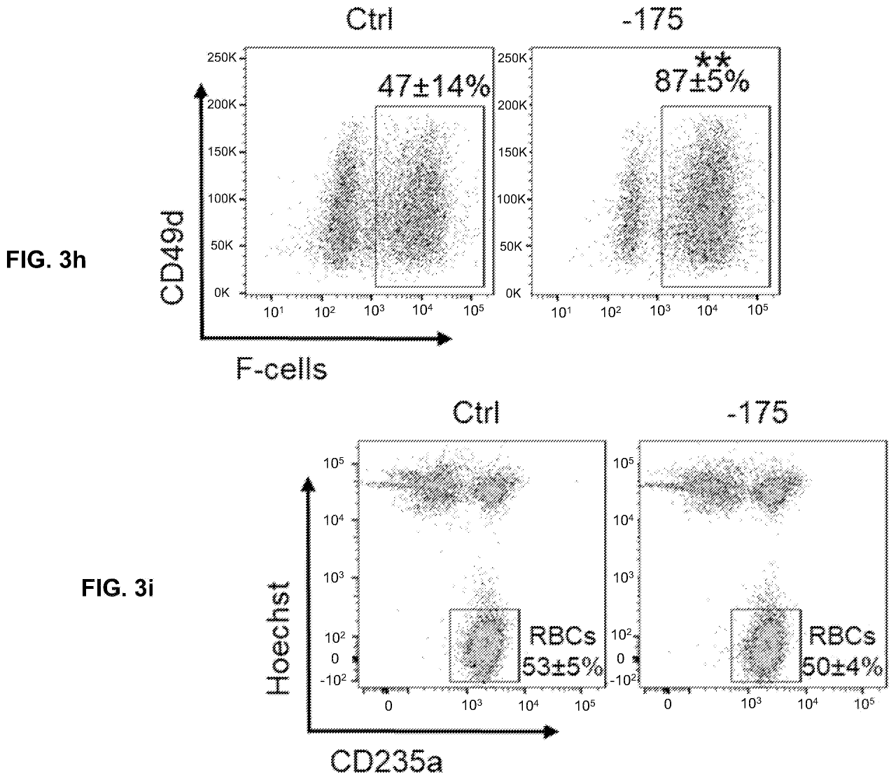

To test the base editing efficiency in CD34+ HSPCs from healthy donors, ribonucleic acid protein (RNP) complexes of base editor and sgRNA were electroporated separately targeting each of these 3 sites in the gamma-globin promoter, using non-targeting sgRNA (NT) as a negative control. An editing efficiency of 9% was observed for the −198 site with ≤1% bystander mutations (FIG. 2. a). For −175 site, the on-target editing was 30% with ≤1% bystander mutations (FIG. 2. b). Using a guide targeting the −113 site, 5% editing was observed at the −112 nucleotide, 20% editing at the −113 nucleotide, and 17% editing at the −116 nucleotide (FIG. 2. c). Further, <1% indels were measured following each of the three editing strategies or when using NT sgRNA (FIG. 2. d). Flow cytometric analysis of in vitro differentiated erythroid cells edited at the −198 site showed an increased population of F-cells from 49±13% in untreated controls to 62±13%. Editing at the −175 and −113 sites yielded increased F-cells, at 88±5% and 82±5%, respectively (FIG. 2. e, g). Each edited population was differentiated into mature erythroid cells over 21 days. Analytical IE-HPLC performed on the lysates of these cells revealed that the treatment targeting the −198 polymorphism raised the HbF expression to 22±0%. the treatment targeting the −175 polymorphism to 54±13%, and the treatment targeting the −113 polymorphism to 37±10% compared to 12±4% in non-edited cells and 12±3% in NT cells (FIG. 2. f). Also, base editing did not alter the expression of erythroid maturation markers Band3 or CD49d (FIG. 2. h). Overall, these results demonstrate that the greatest editing efficiency and HbF induction is obtained by the −175 T→C edit, followed by the −113 and −116 A→G edits, with the −198 T→C edit yield the least efficient editing and HbF induction.ABE7.10 recreates HPFH −175 T→C in SCD donor CD34+ cells and induces HbF in erythroid progeny to inhibit their sickling under hypoxia. To test the anti-sickling properties of induced gamma-globin in a clinically relevant model, the −175 T→C HPFH mutation was edited in the HBG1/2 promoters SCD patient derived CD34+ HSPCs. On-target base editing efficiency achieved values of 25%, 34% and 48% on day 3, 6 and 9, respectively, following electroporation (FIG. 3. a) with low frequency of indels <1% (FIG. 3. a, g). Flow cytometric analysis of edited cells revealed that after editing, the proportion of F-cells increased to 87±14% compared to 47±5% in non-edited cells (FIG. 3. b, h). IE-HPLC revealed that −175 site T→C mutation raised the HbF content to 51±4% compared to 12±5% in non-edited cells (FIG. 3. c). Base editing did not alter the expression of erythroid maturation markers Band3 or CD49d (FIG. 3. d) or alter erythrocyte enucleation (FIG. 3. i). To assess the effect of HbF reactivation on the sickling phenotype, an in vitro deoxygenation assay was performed that induces sickling of erythrocytes under hypoxia. After being subjected to hypoxia (2% O2) for 8 hours, 43±8% of control erythroid cells exhibited sickled morphology as compared with 18±5% of base edited cells (FIG. 3. e, f). These results demonstrate that creation of de novo binding site for TAL1 in the HBG promoters leads to reactivation of HbF sufficient to revert the sickling phenotypes in erythrocytes differentiated from erythroid progenitors derived from SCD patients.

### ABE7.10 Generates HPFH Mutations in Normal Human Hematopoietic Stem Cells and Induces HbF Levels in Erythroid Progeny In Vivo

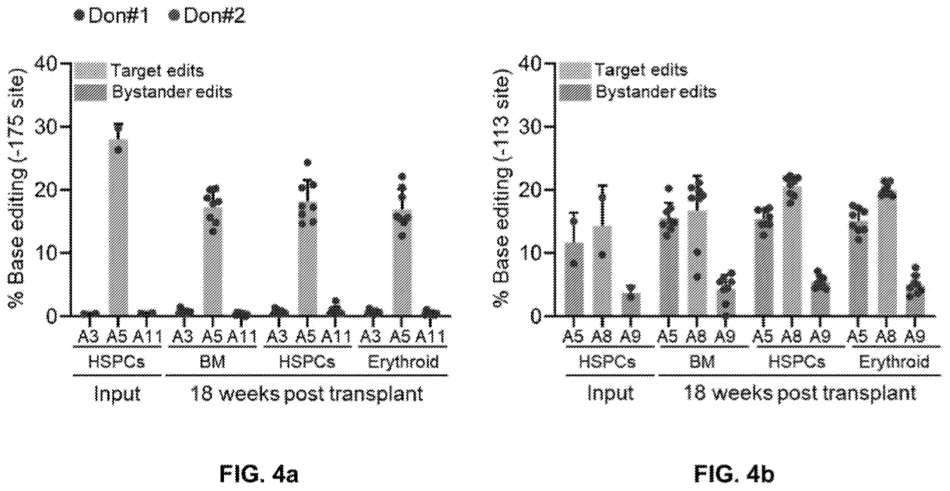

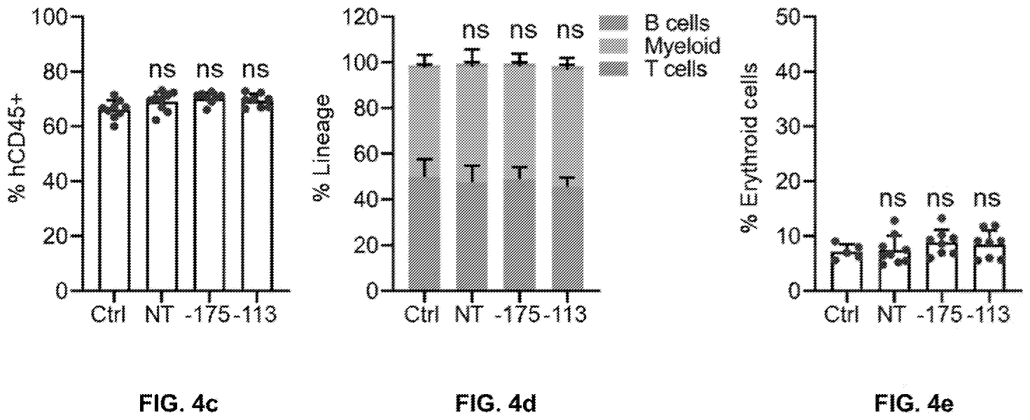

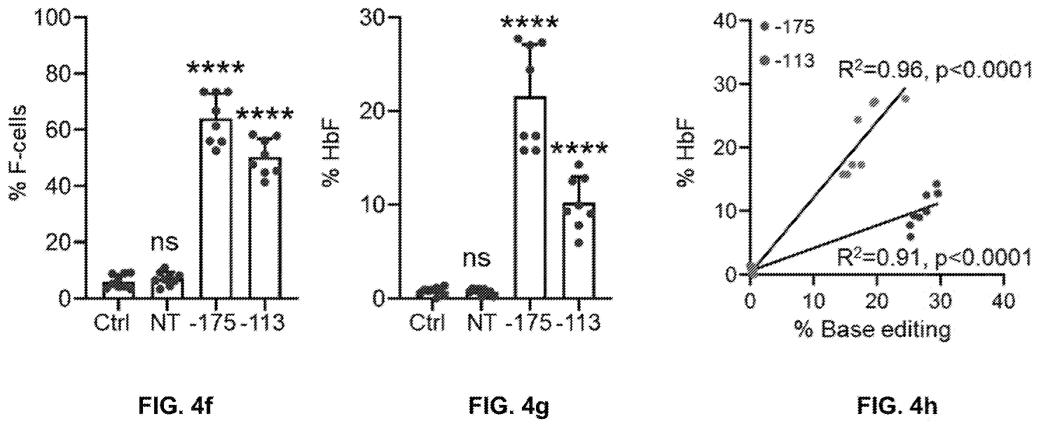

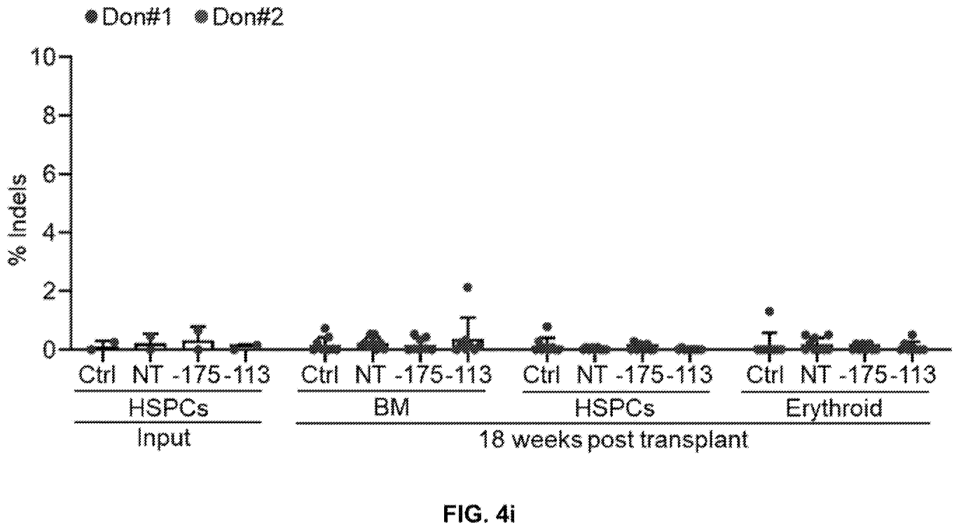

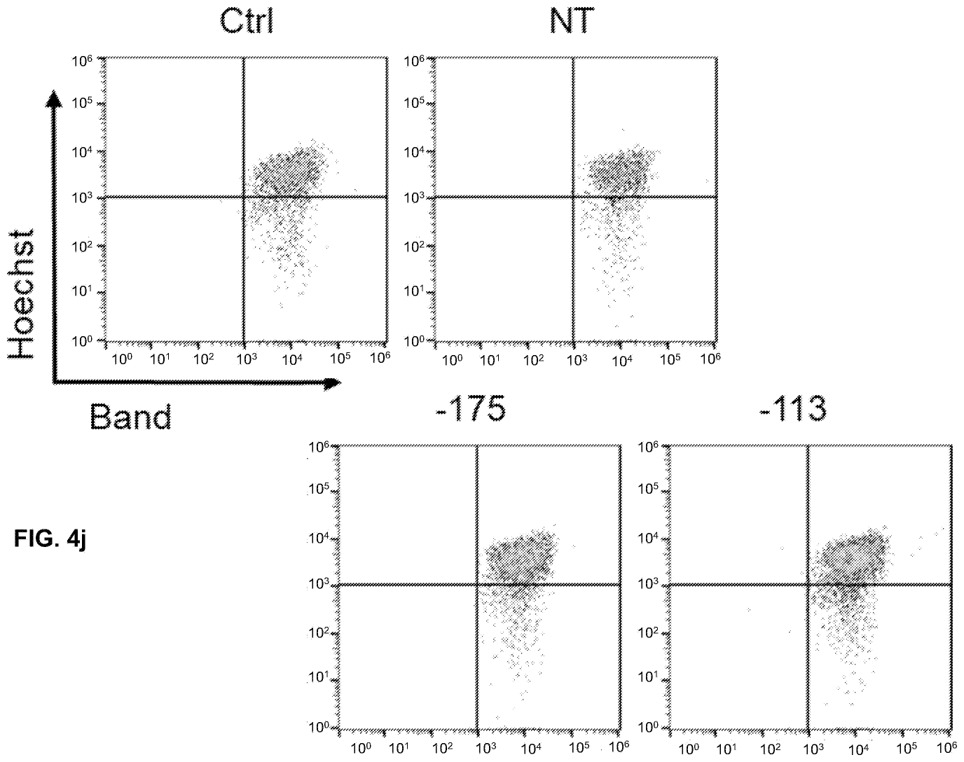

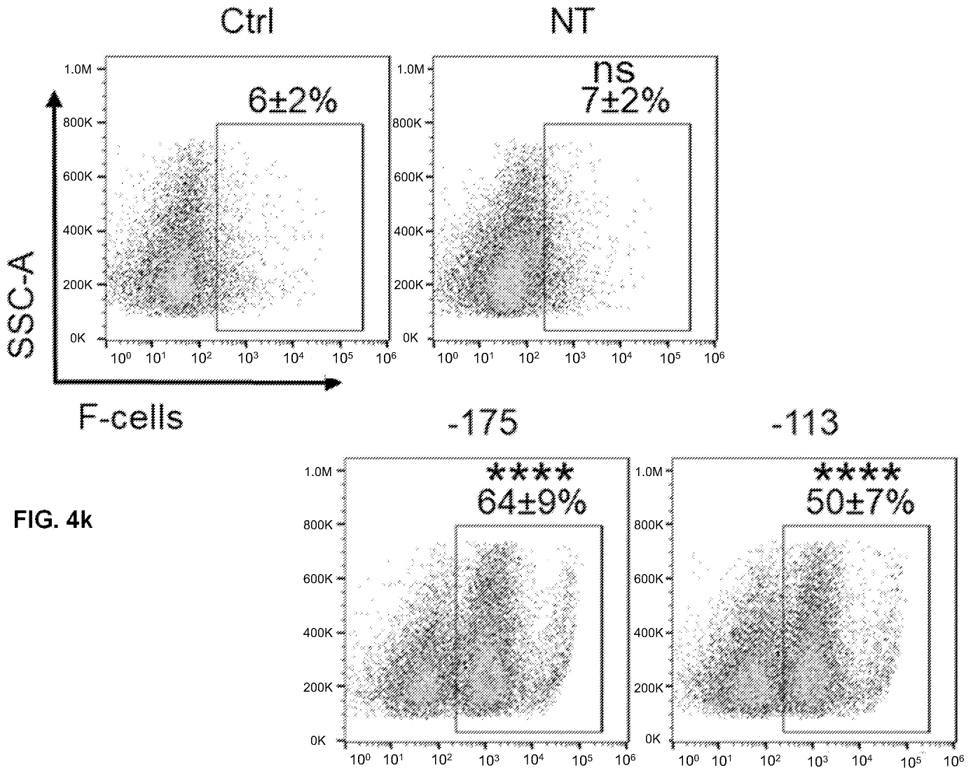

To assess the capacity to edit repopulating HSCs, −175 T→C and −113 A→G edited CD34+ cells were transplanted into nonobese diabetic/severe combined immunodeficiency/I12rgamma−/−/KitW41/W41 (NBSGW) mice. The fraction of engrafted human cells and their editing frequencies were measured periodically in extracted blood, and after the mice were euthanized at 18 weeks, from bone marrow. The base editing frequencies at the −175 site of edited human donor in bone marrow declined from 28% after editing to 18% at 18 weeks after xenotransplantation (FIG. 4. a). Editing frequencies were also similar in HSPCs and erythroid cells derived from them, suggesting that repopulating HSCs were edited at an allelic efficiency of 18% (FIG. 4. a). At the −113 site, the editing frequencies measured after 18 weeks of engraftment was 17% at the −113 nucleotide and 16% at the −116 nucleotide, slightly higher than the editing measured in the population before engraftment, which was 14% and 12%, respectively (FIG. 4. b). Also, the editing frequencies were similar in HSPCs and erythroid cells derived from bone marrow, suggesting that repopulating HSCs were edited at an allelic efficiency of approximately 17% (FIG. 4. b). The base editing spectrum in edited cells was similar before and after transplantation, with no clonal dominance in the latter populations (FIGS. 4. a and 4. b). Indels frequencies for both −175 and −113 sites were <1% and similar to that of cells before engraftment (FIG. 4. i). No significant differences in human/mouse chimerism occurred between edited and nonedited donor derived CD45+ hematopoietic cell populations (FIG. 4. c). Chimerism levels were similar for edited and nonedited donor T cells (hCD45+/CD3+), B cells (hCD45+/CD19+), myeloid cells (hCD45+/CD33+) (FIG. 4. d), and erythroid cells (CD45-/CD235a+) in bone marrow at 18 weeks (FIG. 4. e).

Circulating human RBCs are short lived and difficult to detect in mouse xenotransplants. Therefore, immune-selected CD235a+ donor-derived erythroblasts were studied from the bone marrow of recipient mice at 18 weeks after transplantation. Mainly late stage erythroblasts and reticulocytes were obtained. with no obvious differences in expression of maturation markers between the edited and control and non-target populations (FIG. 4. j). Notably, background % HbF was lower in control (nonedited) erythroblasts generated in vivo compared with in vitro cultures (compare FIG. 2. f to FIG. 4. g). The percentages of both HbF-immunostaining cells (F cells) and HbF protein compared with overall Hb were significantly increased in edited vs control and non-target erythroid cells generated in vivo (FIG. 4. f, g, k). For the −175 site, considering that the HbF content was 22%±5% in erythroblasts with ˜18% base editing (FIG. 4. a, g). it is likely that HbF was induced in all edited cells, thereby favoring therapeutic efficacy. However, for the −113 site, HbF content was 10±3% in erythroblast with ˜20% base editing (FIG. 4. b, g) suggesting that −175 edit more robustly induced HbF compared to −113/ −116 edits. The HbF content in erythroid cell lysates correlated with editing frequency based on measurements from individual mice (FIG. 4. h). These xenotransplantation studies demonstrate that it is feasible to induce erythroid HbF expression by ABE7.10 RNP-mediated recreation of HPFH mutations to recruits TAL1 or GATA1 to the HBG1/HBG2 promoters, with no obvious alterations in hematopoietic development in vivo.

ABE7.10 Generates −175 T→C HPFH Mutation in SCD Donor Derived Human Hematopoietic Stem Cells. And Induces HbF Levels in Erythroid Progeny In Vivo

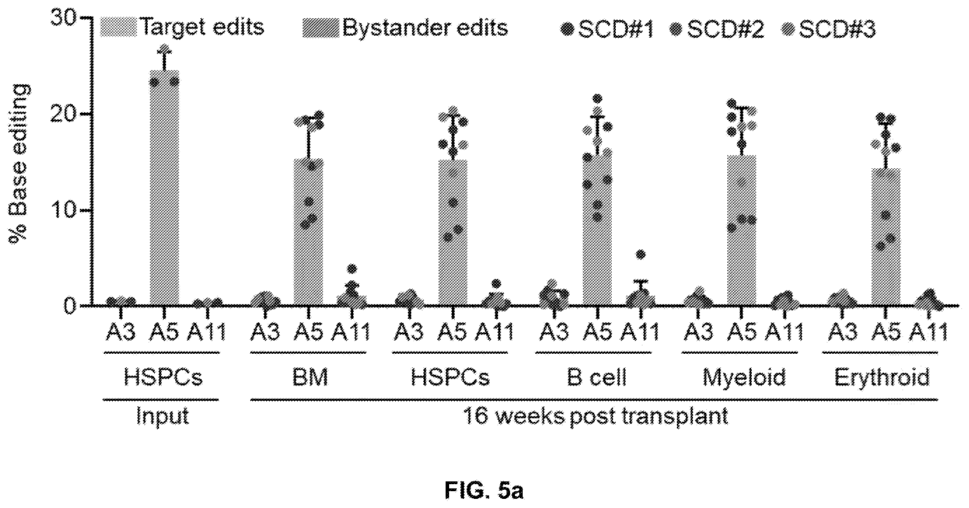

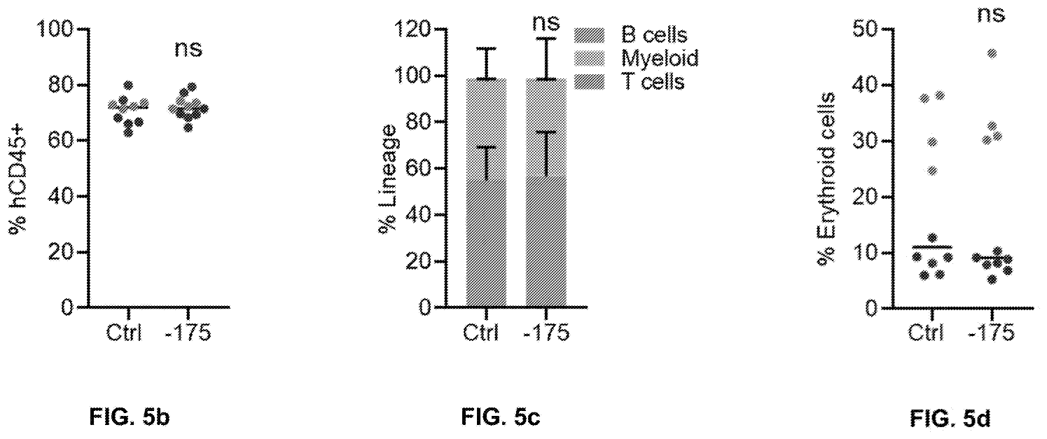

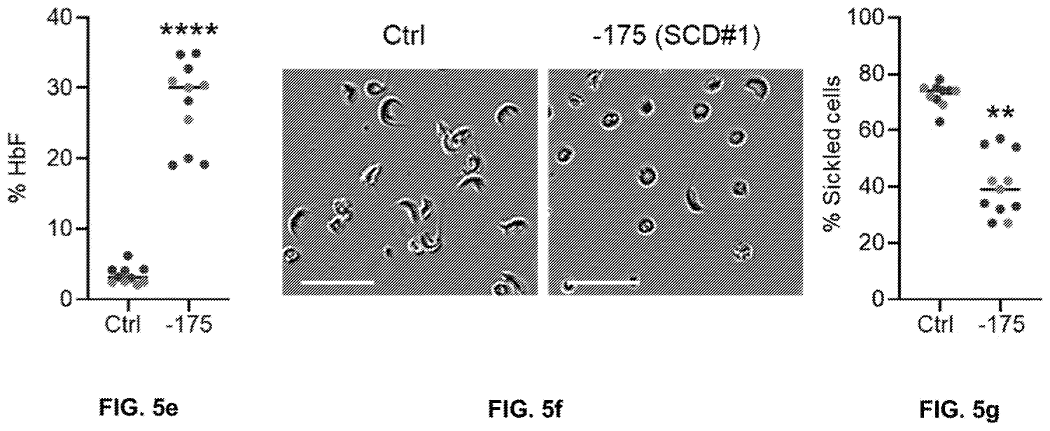

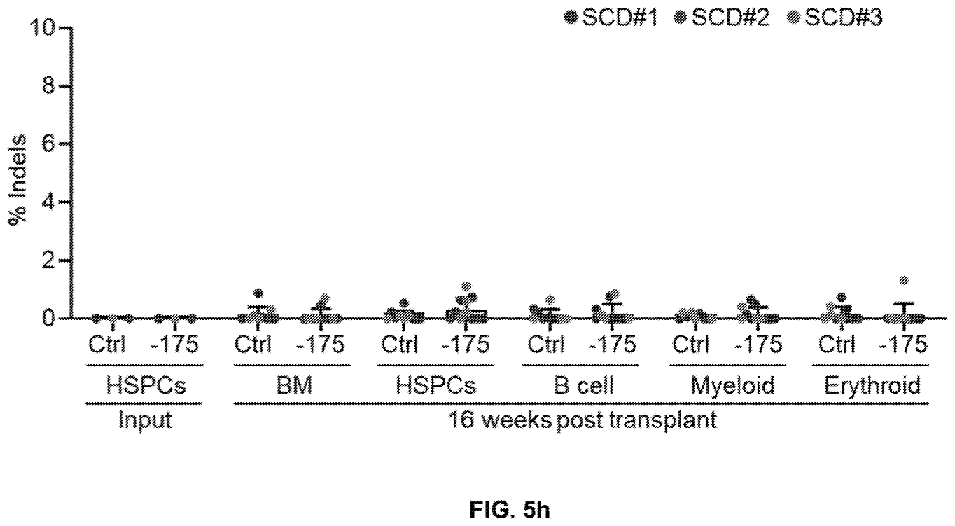

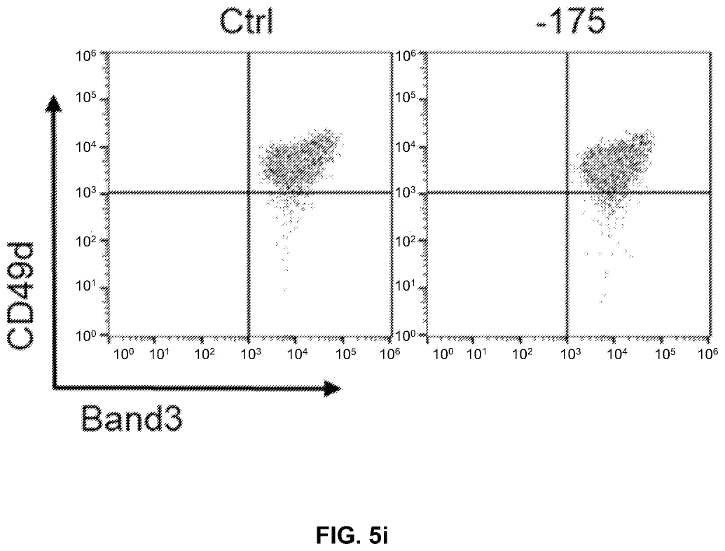

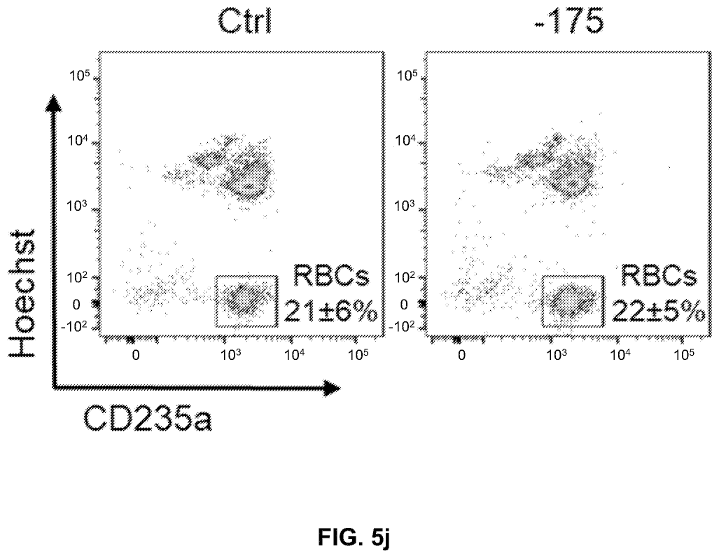

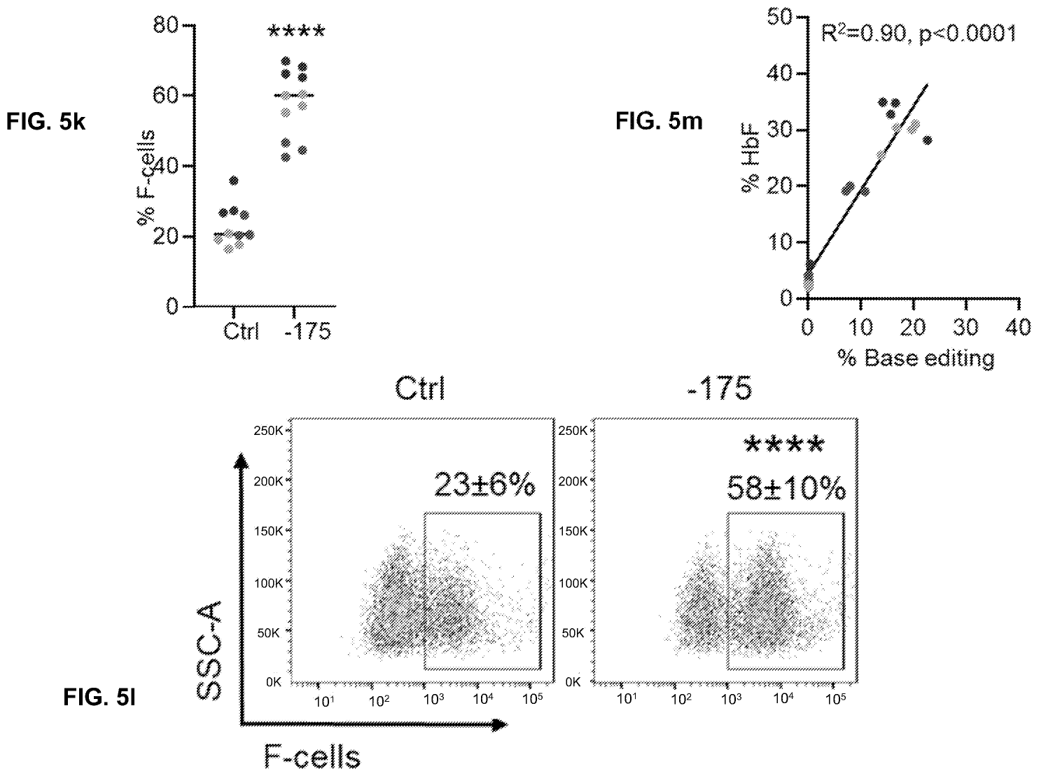

It was next assessed whether SCD patient repopulating HSCs, the target cell population for autologous stem cell therapy, could be base edited to correct long-term phenotypes. Base editor RNPs targeting the −175 variant were electroporated into plerixafor-mobilized peripheral blood CD34+ HSPCs from individuals with SCD. The editing frequency before transplantation was 25% on day 3 post electroporation and 15% (60% of input cells) at 16 weeks after transplantation (FIG. 5. a). Editing frequencies were also similar in CD34+ donor cell-derived myeloid, erythroid, and B cells (FIG. 5. a), indicating that editing did not alter the development of these lineages from HSCs. The spectrum of on-target base edits in SCD CD34+ HSPCs was similar to that observed after editing healthy CD34+ HSPCs and did not change appreciably after xenotransplantation (FIG. 5. a). After xenotransplantation into NBSGW mice, edited and control CD34+ cells populated the bone marrow similarly and gave rise to similar fractions of human T, B, myeloid, and erythroid cells (FIG. 5b-d). Indels frequencies were <1% and was similar to that of input (FIG. 5. h).

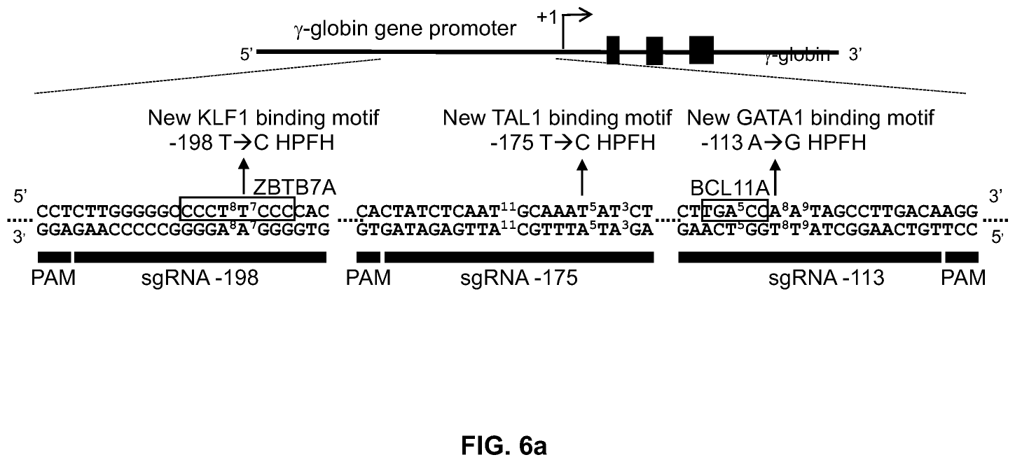

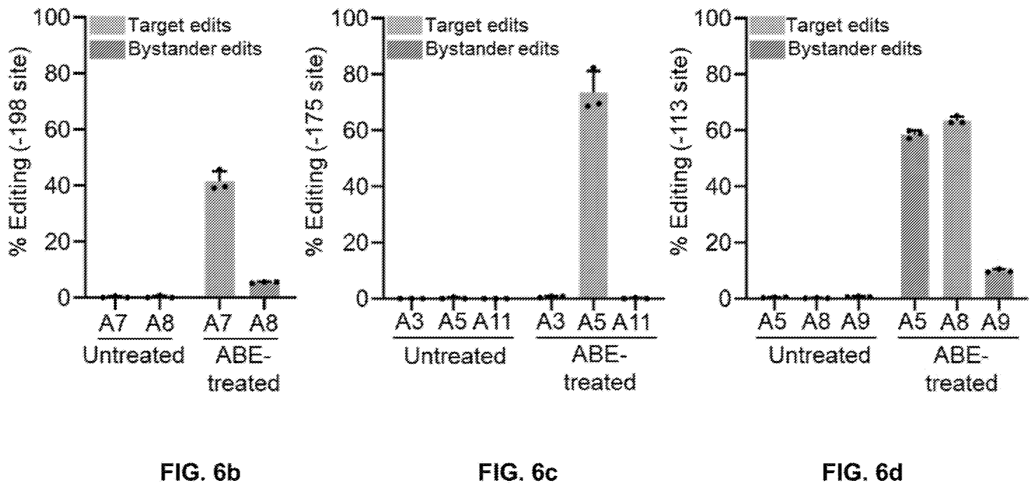

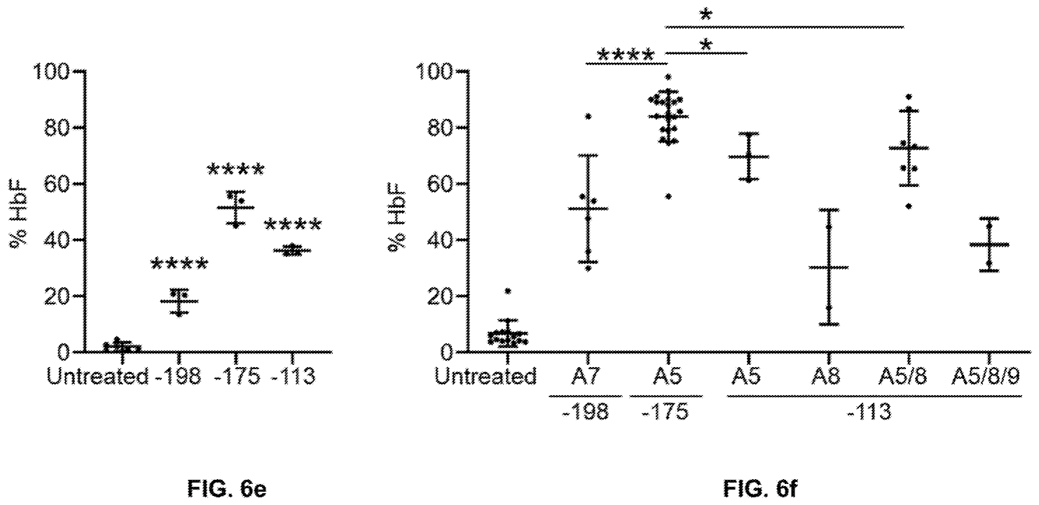

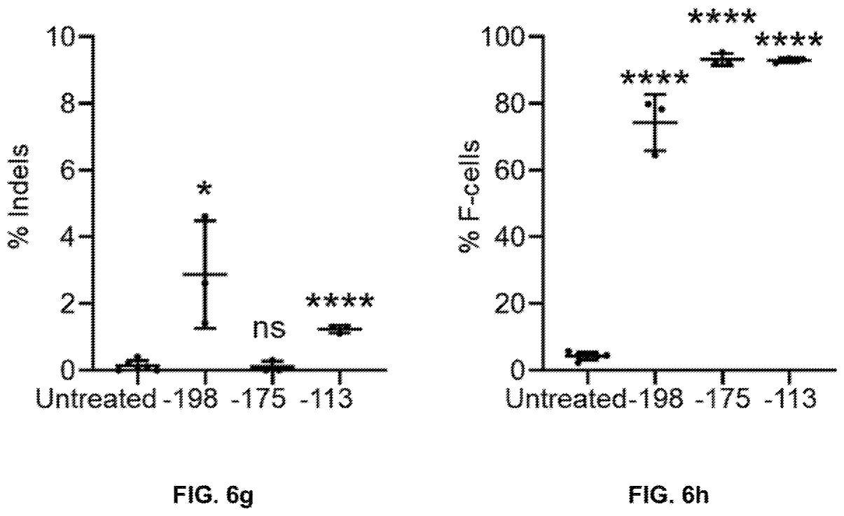

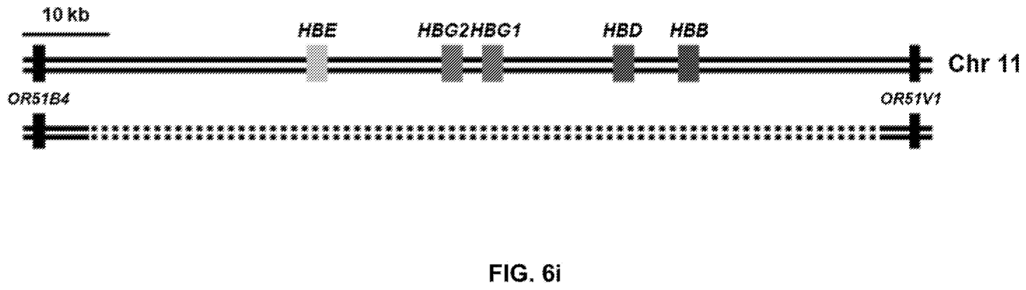

16 weeks after transplantation, bone marrow erythroblasts derived from gene-edited and control CD34+ HSPCs exhibited similar maturation profiles and enucleation as determined by flow cytometry (FIG. 5. i, j). Erythroblasts derived from gene edited HSPCs CD34+ cells contained 58±10% F cells as compared with 23±6% in control nonedited erythroblasts (FIG. 5. K, l). The HbF content was 28±6% after gene editing vs 3±1% in control erythrocytes (FIG. 5. c). HbF induction in SCD erythroid cells correlated with base edit formation (Supp. FIG. 6f). similar to observations in erythroid cells derived from normal donor HSPCs (FIG. 5. m). An in vitro sickling assay on erythroid cells purified from bone marrow indicates robust reduction of sickling in edited cells (40±11%) compared to control cells (73%±4%) (FIG. 5. f, g). Thus, HbF induction resulting from base editor RNP-induced recreation of the naturally occurring −175 mutation in the HBG1 and HBG2 gene promoters inhibits erythroid cell sickling.

### Discussion

Developing effective approaches to raise HbF for b-hemoglobinopathies has been a “holy grail” in the field for more than 50 years. A major breakthrough in the field came from genome-wide association studies (GWAS) showing that variations in the extended beta-like globin gene cluster, BCL11A, and the HBS1L-MYB intergenic region are associated with RBC HbF levels. In embodiments of this disclosure, ABEs are used to generate naturally occurring point mutation in HBG promoter that cause HbF production.

First, the proof of concept was recapitulated to recreate the HPFH mutations in HBG promoter to induce HbF in adult erythroid progenitor cell line. Precise point mutations without DNA double strand break were achieved by base editors with low rate of indels. Although the rate of editing different for each guide all three HPFH mutations induced significant levels of HbF. Very low rate of by-stander mutation was observed within the editing window for sgRNA −198 and −175, but −113 guide exerts significant by-stander mutations with one mutation (position G5) in BCL11A binding motif (TGACC) without impeding the HbF induction. Clonal analysis in hemizygous HUDEP-2 cells displayed significant HbF production for all three HPFH mutations. Interestingly, the by-stander mutation at G9 position in −113 guide along with other mutations comparatively reduced the HbF levels suggest that this mutation affects the binding of GATA1 or BCL11A to its corresponding motifs. Notably, −175 T→C mutation had robust HbF induction compared to other 2 mutations.

Second, a measurable precise HPFH point mutations in human primary HSPCs was created using base editors. Editing rate for −175 guide is higher (40%) compared to other 2 sites. HbF induction is correlated with the editing rates in erythroid progeny for all 3 sites and a robust HbF induction was observed for −175 T→C mutation similar to HUDEP-2 cells. In vivo studies in NBSGW mice model revealed that editing frequency was retained up to 20% in long term repopulating HSCs and induced up to 22% HbF in erythroid progeny. Base editing of HSPCs doesn't skew the erythroid maturation or enucleation of terminal differentiated erythroid cells.

Third, by using gRNAs targeting the −175 site, a robust, pancellular HbF reactivation and a concomitant reduction in b^{S}-globin levels was achieved, recapitulating the phenotype of asymptomatic SCD-HPFH patients. The HbF induction (>60%) observed in the RBCs derived from edited erythroid progenitors is sufficient to ameliorate the SCD phenotype. In vivo experiments demonstrated that editing frequency was maintained up to 20% 16 weeks post transplantation and induced HbF at therapeutic level (˜30%) without affecting the erythroid population. kinetics and enucleation. Therapeutic levels of fetal hemoglobin were observed in SCD erythroid progeny with the −175 T→C edit, which was the most attractive of three sites assessed. An autologous transplantation following this edit in mobilized patient HSPCs serves as an attractive new therapeutic. This strategy may be used to treat both beta-thalassemia in addition to SCD, as elevated HbF induction compensates for the deficient beta-globin chains. In summary, the present disclosure demonstrates the proof of concept using a novel adenosine base editing approach to precisely install a naturally occurring HPFH mutations in CD34+ HSPCs that leads to SCD resistance. Therapeutic levels of fetal hemoglobin were observed in SCD erythroid progeny with the −175 T→C edit, which was the most attractive of three sites assessed. An autologous transplantation following this edit in mobilized patient HSPCs serves as an attractive new therapeutic. This also paves the way to explore the new ABE editing strategies/targets in CD34+ HSPCs to cure SCD, like multiplex editing or novel targets.

### Example 2

### Adenosine Base Editing Potently Induces Fetal Hemoglobin and Inhibits Erythroid Sickling

Beta-hemoglobinopathies (e.g., anemia, beta-thalassemia, and sickle cell disease (SCD)) are the most common human monogenic disorders and affect millions worldwide. Augmentation of fetal hemoglobin (HbF) by manipulating DNA regulatory elements represents a promising approach to treat beta-hemoglobinopathies. As disclosed herein, adenosine base editors (ABEs; see Table 4 and Table 5) were used to compare three naturally occurring HPFH mutations in gamma-globin gene promoters: −113 AàG, −175 TàC, and −198 AàG. Each edit upregulated expression of gamma-globin in an erythroid progenitor cell line. The −175 TàC was identified as a potent HbF inducer, and was pursued in SCD patient derived adult CD34+ HSPCs. Edited cells persisted for over 16 weeks after engraftment into immunocompromised mice and demonstrated an induction of fetal hemoglobin, reduction in sickling propensity, and maintenance of a stem cell state. This method of gamma-globin-proximal base editing is an attractive strategy to generate healthy autologous cellular populations for treatment of sickle cell disease (SCD) and beta-thalassemia.

TABLE 4
ABEs with concentrations used for site editing

| S. | | Type of | Concentration | | |
|---|---|---|---|---|---|
| No | Name | reagent | Used | | PAM |
| 1 | ABE max-SpCas9 (7.10) | mRNA | 2 | ug/uL | NGN |
| 2 | ABEmax-SpCas9 (7.10) | Protein | 5 | uM | NGN |
| 3 | ABE8e-SpCas9 | mRNA | 2 | ug/uL | NGN |
| 4 | ABE8e-SpCas9 | Protein | 5 | uM | NGN |
| 5 | ABE8 SpRY-Sp Cas9 | mRNA | 2 | ug/uL | NRN/NYN |

TABLE 5
ABEs used for site editing

| | | Type of | |
|---|---|---|---|
| S. No | Name | reagent | PAM |
| 1 | ABE max-SpCas9 (7.10) | mRNA | NGN |
| 2 | ABEmax-SpCas9 (7.10) | Protein | NGN |
| 3 | ABE8e-SpCas9 | mRNA | NGN |
| 4 | ABE8e-SpCas9 | Protein | NGN |
| 5 | ABE8 SpRY-SpCas9 | mRNA | NRN/NYN |
| 6 | ABE8 SpRY-SpCas9 | Protein | NRN/NYN |

Naturally occurring rare variants at the gamma-globin gene (HBG2 and HBG1) promoter causes sustained fetal hemoglobin (HbF) production in adults, a condition termed as nondeletion types of hereditary persistence of fetal hemoglobin (ndHPFH). Co-inheritance of ndHPFH in patients with sickle cell disease or beta-thalassemia cause elevated levels of HbF in red blood cells (RBCs) and ameliorate the clinical severity. Multiple ndHPFH mutations have been reported at proximal promoter of G-gamma- and or A-gamma-globin gene and fall into three distinct clusters; −200 HPFH cluster, −175 site and −115 HPFH cluster. HPFH variants at −115 and −200 HPFH clusters disrupt the binding of transcriptional repressors (BCL11A and ZBTB7A) at gamma-globin promoter resulting in activation of HbF. Three HPFH variants that include-198 T→C, −175 T→C and −113 A→G creates the de novo binding site for transcriptional activators KLF1, TAL1 and GATA1 respectively and activate gamma-globin gene.

Genome editing via CRISPR/Cas9 or related nucleases represents a promising approach to cure beta-hemoglobinopathies. Recently, it has been demonstrated that CRIPSR/Cas9 mediated disruption of BCL11A and ZBTB7A binding sites at gamma-globin promoter raised RBC HbF. Additionally, several groups showed that genome editing to disrupt an erythroid-specific BCL11A enhancer in HSPCs induces RBC HbF to therapeutic levels. As HBG1 and HBG2 are nearly identical and arranged in tandem, targeting the BCL11A or ZBTB7A binding site resulted in simultaneous DSBs in both gene promoters, with 9 to 20% of cells exhibiting a deletion of the intervening 4.9 kb region. In addition, simultaneous DSBs can result in loss or inversion of the intervening genetic material and/or chromosomal rearrangements. It has been reported that on-target genome editing via nucleases can also lead to chromothripsis. These products of Cas9 nuclease cleavage can evoke cytotoxic DNA damage responses. Furthermore, due to the variability of possible insertions/deletions (indels) created from DSB mediate non-homology end joining (NHEJ), the productive indels that disrupt the repressor binding can vary. leading to inconsistent levels of HbF per cell. Although the BCL11A-mediated repression of fetal hemoglobin can be disrupted either by removing its binding site in the fetal hemoglobin promoters or by disrupting the BCL11A gene itself, neither method has led to full restored expression of HbF, with even >90% editing efficiency leading to only 30-40% HbF. A more potent editing strategy to switch expression from adult beta-globin to gamma-globin may improve patient outcomes in the long-term.

Base editors are precise CRISPR/Cas9-derived tools that can install desired transition mutations without generating double stranded DNA breaks (DSBs), potentially alleviating safety risks and increases versatility. There are currently two main flavors of base editors consist of a Cas9 nickase fused with either a cytosine (CBE) or adenine (ABE) deaminase, which can in turn convert cytosines to thymines or adenosines to guanines, respectively without creating DSBs. A new base editor was recently reported to effectively convert the sickle cell mutation to a benign non sickling Makassar variant with high efficiency in patient-derived HSCs and in mice. However, while this approach is feasible for the treatment of SCD, it cannot treat beta-thalassemia. Inducing fetal hemoglobin can potentially treat both diseases, so the identification of an editing strategy to potently induce HbF is desirable. Recent studies demonstrated that base editors induces HbF expression by introducing edits in the erythroid-specific BCL11A enhancer or in the gamma-globin promoter. In contrast to disrupting repressor binding sites with nucleases. three naturally occurring HPFH variants were identified, −198 T→C (KLF1 binding site), −175 T→C (TAL1 binding site), and −113 A→G (GATA1 binding site) base pairs upstream of the gamma-globin gene transcription start sites to recreate using ABEs. All these regions represent target sites for induction of HbF by genome editing. The −175 T→C edit more potently induced fetal hemoglobin relative to similar strategies at the −113 A→G and −198 T→C positions of the fetal hemoglobin promoters. The creation of a de novo TAL1 binding site at the −175 site was demonstrated to be more potent than the disruption of the BCL11A binding site in inducing fetal hemoglobin.

### A Base Editor (ABE7.10) Recapitulates HPFH Mutations and Induces HbF in HUDEP-2 Cells.

Synthetic single guide RNAs (sgRNAs) were designed targeting the HPFH mutations at the −198, −175, and −113 positions relative to the transcription start site of the gamma-globin gene promoters (FIG. 6a; Table 3). The −198 and −113 sites can be targeted using ABE7.10 with the canonical NGG PAM and while the −175 site required the use of the new ABE7.10 with the NG targeting PAM. Electroporation of sgRNAs and ABE mRNA into human umbilical cord blood-derived derived erythroid progenitor (HUDEP-2) cells revealed different on-target and bystander editing efficiency for all 3 sites. At the −198 site, the editing frequency of the target variant (A7) of the protospacer was 41% with a bystander mutation (A8) occurring in ≤5% of reads as measured by high-throughput sequencing (HTS; FIG. 6b). At the −175 site the editing frequency of the target variant (A5) of the protospacer was 73% with bystander mutations at positions A3 and All occurring in ≤1% of reads (FIG. 6c). Third, at the −113 site the editing frequency of the target variant at position 8 of the protospacer was approximately 59% with bystander mutations at positions A5 and A9 occurring with 64% and 10% efficiency, respectively (FIG. 6d). Low rates of indels were observed following editing: 3% at the −198 site and ≤1% at both the −175 and −113 sites (FIG. 6g). The base edited HUDEP-2 cells were differentiated into mature erythroblasts to assess HbF induction following each of the three separate treatments. Flow cytometric analysis of cells edited at −198, −175, and −113 sites revealed a high frequency of HbF-expressing cells (F cells) 74±8%, 93±2%, and 93±1%, respectively, compared to 5±1% in non-edited cells (FIG. 6h). Analytical ion-exchange HPLC (IE-HPLC) was performed on lysates of mature erythrocytes to assess the fraction of HbF protein out of total beta-globin in these cells. In cellular populations edited at the −198, −175, and −113 sites, HbF content was measured at 18±4%, 52±6%, 36±1%, respectively, compared to 2±1% in non-edited cells (FIG. 6c). The results suggest that the −175 T→C edit, which creates a binding site for TAL1, is more potent for inducing HbF than the other tested variants.

Next, single-cell clones were isolated from hemizygous HUDEP-2 cells (harboring a single copy of the HBG2 and HBG1 gene opposite a deletion on the sister chromosome) (FIG. 6i) following base editing and cultured them in erythroid liquid culture to assess the HbF induction resulting from editing outcomes. Clones with HPFH mutations at both the gamma-globin genes (HBG2 and HBG1) were picked for HPLC analysis. HbF expression increased to 51±19% of total beta-like globins with the −198 TàC edit, 84±9% for the −175 TàC edit, 30±20% for the −113 edit alone, 70±8% for the −116 edit alone, and 73±13% in cells with edits at both the −113 and −116 positions (FIG. 6f). Clones with all three of the −112, −113 , and −116 AàG edits had decreased HbF expression of 38±9% (FIG. 6f). Overall, these results suggest that recreation of HPFH mutations in the gamma-globin proximal promoter induced HbF in erythroid cells. The −175 TàC polymorphism, which creates a de novo binding site for an erythroid transcription factor TAL1 showed greater induction of HbF than the other HPFH mutations. Interestingly, the −116 AàG polymorphism that disrupts a BCL11A binding site led to even greater HbF induction relative to −113 AàG that creates a GATA1 binding site, while the combination of both edits yielded a synergistic effect on gamma-globin expression. The addition of a third −112 AàG edit decreased the levels of HbF induction, likely due to the disruption of the new GATA1 binding motif created by the −113 edit.

### The 175T→C Mutation Creates a De Novo Binding Site for TAL1 and Requires GATA1 for Active Binding.

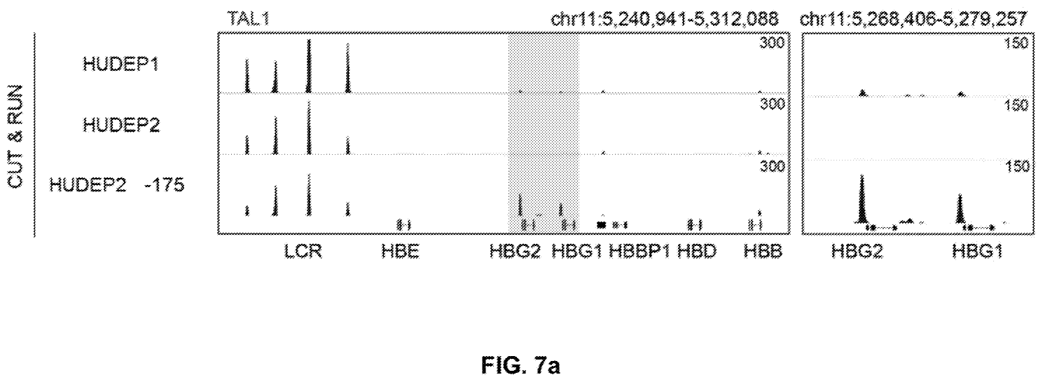

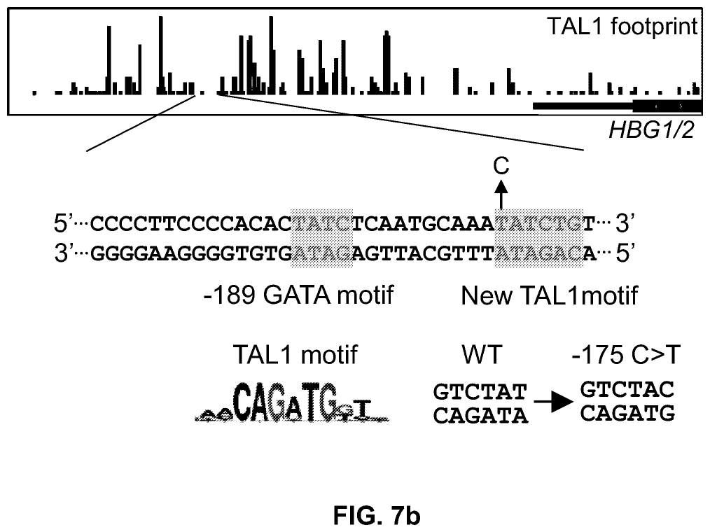

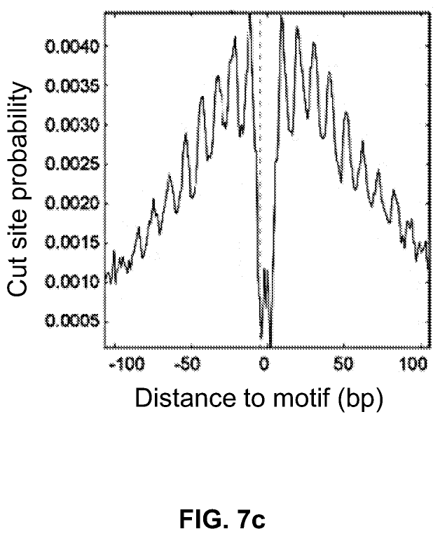

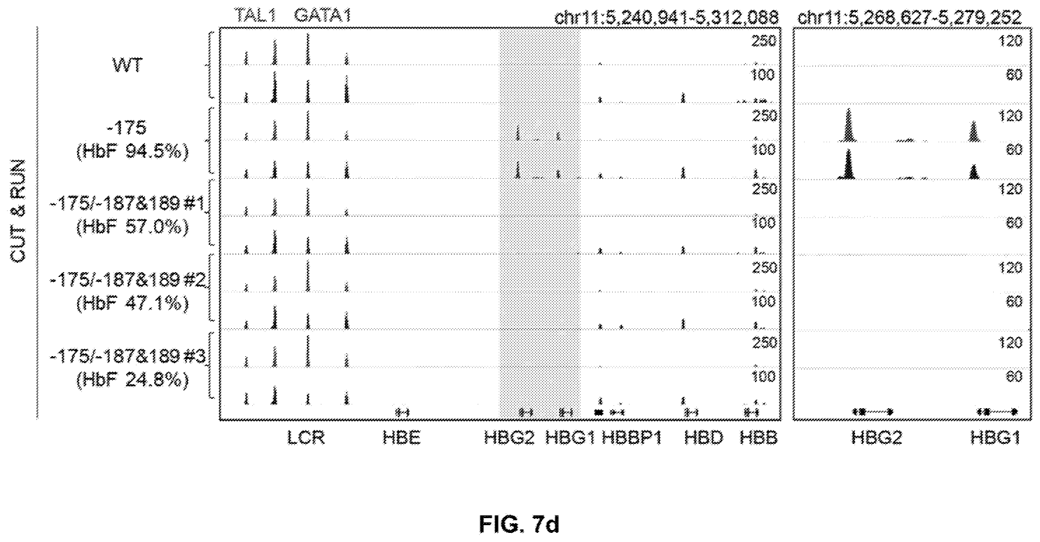

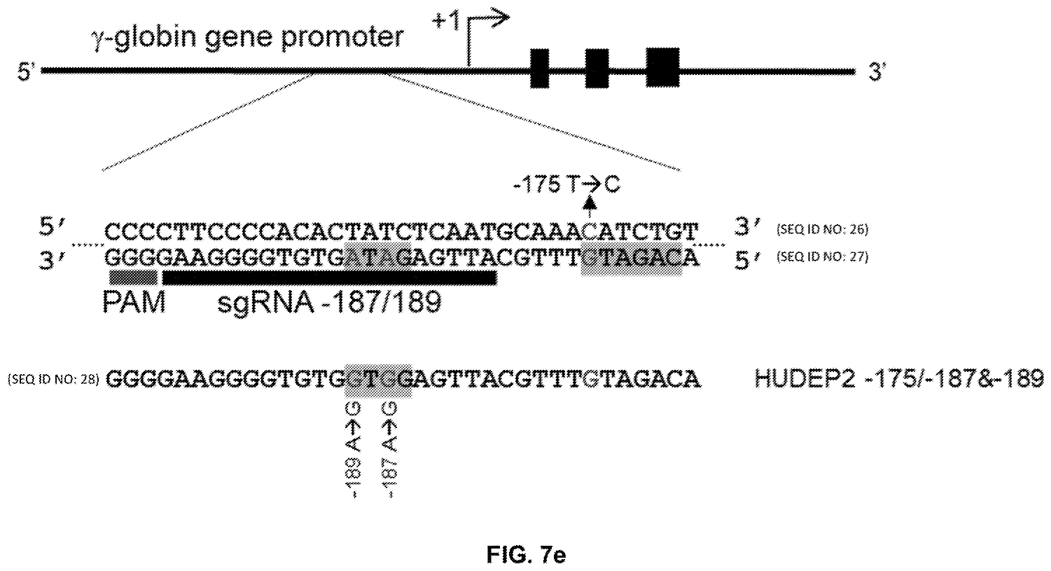

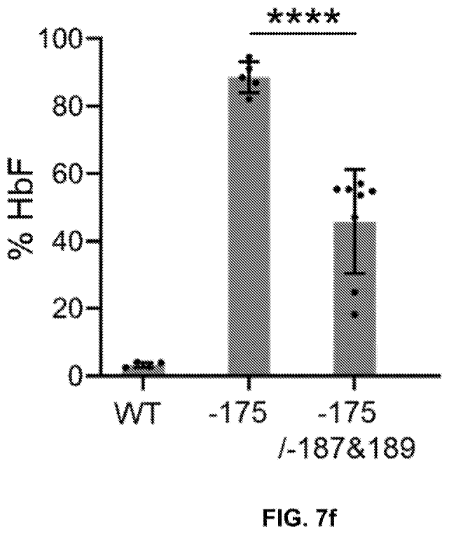

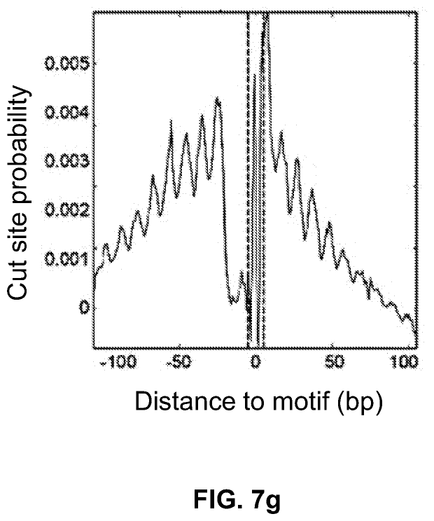

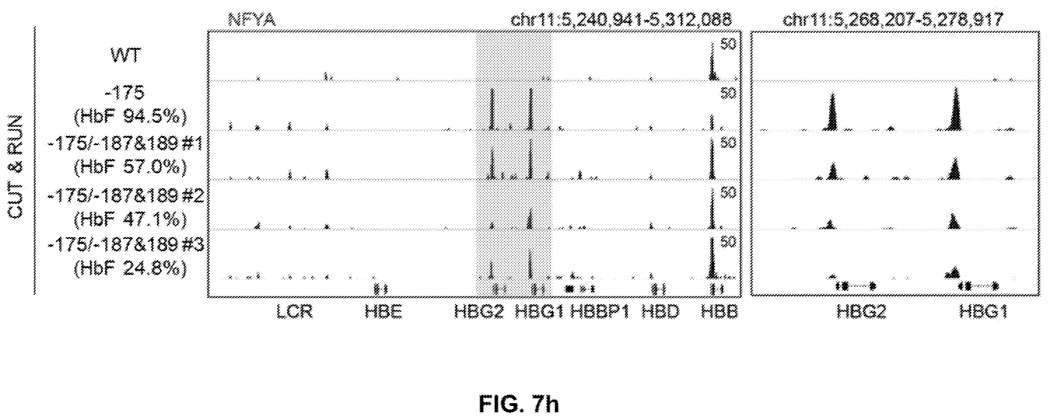

It was further investigated whether HUDEP-2 with −175 TàC variant recruits TAL1 to HBG1/2 promoter by performing CUT&RUN assay, a potential method to detect native DNA-protein interactions. As expected, TAL1 chromatin occupancy at HBG1/2 promoter were relatively high in HUDEP-2 clone with −175 TàC and no binding of TAL1 in wild type HUDEP-2 cells (FIG. 7a). The TAL1 footprint analysis suggests that HBG1/2 promoter TAL1 binding region contains a closely spaced GATA motif within 10 base pair window that was resistant to micrococcal nuclease digestion. consistent with protection via bound TAL1 (FIG. 7b&c). To test GATA motif functionally, disrupting point mutations were created using the ABE7.10 to convert A→G at positions −187 and −189 relative to HBG transcription start site and generated HUDEP-2 clones with −175 TàC with disrupted GATA motif A→G at positions −187 and −189 A→G (here after double mutant clone; FIG. 7g). The % HbF in double mutant clones was significantly reduced (89±5%) compared to −175 T→C clones (50±15%; FIG. 7h). CUT&RUN analysis in double mutant clones showed a loss of GATA1 and TAL1 binding in double mutant clones and not in −175 TàC clone (FIG. 7d). GATA1 footprint analysis clearly suggests that TAL1 binding region were digested by micrococcal nuclease digestion but proximal GATA motif were protected via bound GATA1 (FIG. 7c). However, HbF were observed to still persists in double mutant clones despite GATA and TAL1 loss (FIG. 7d). CUT&RUN analysis revealed that NFYA binding was still binding at HBG1/2 promoter despite loss of GATA1 and TAL1 binding is a potential reason for persistence of HbF in double mutant clones (FIG. 7d). Together, these findings demonstrate that induction of gamma-globin expression in −175 TàC variant by recruitment of TAL1 requires proximal GATA motif. Also, GATA1 and TAL1 complex recruits NFYA for activation of gamma-globin expression and HbF persists after loss of GATA1 and TAL1.

ABE7.10 Recreates HPFH Mutations in Normal Donor CD34' Cells and Induces HbF in their Erythroid Progeny.

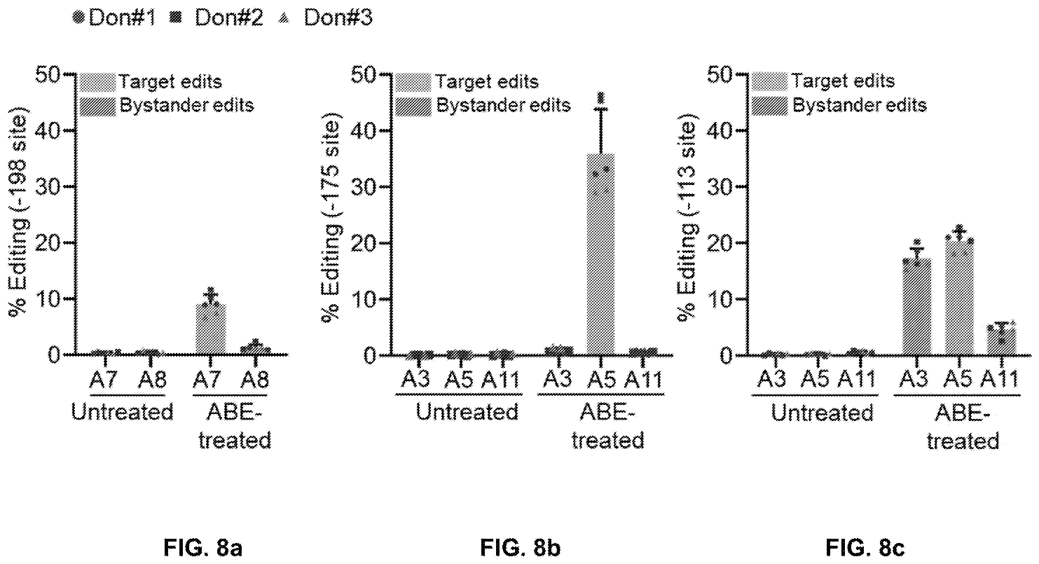

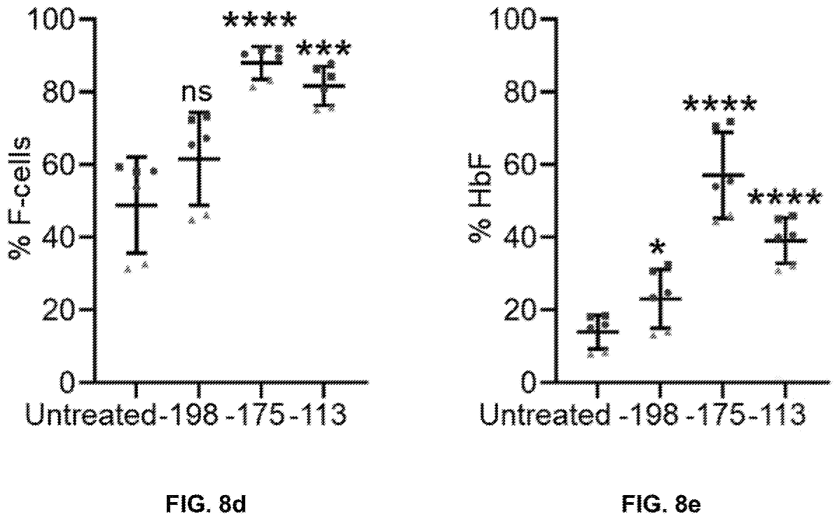

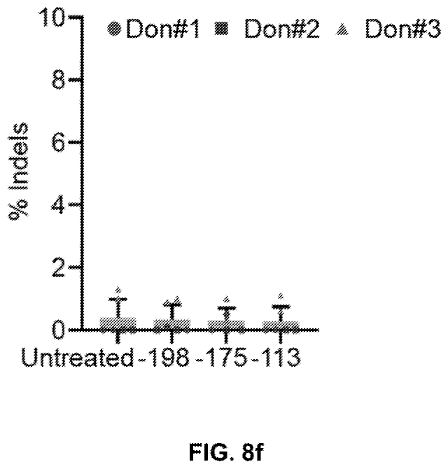

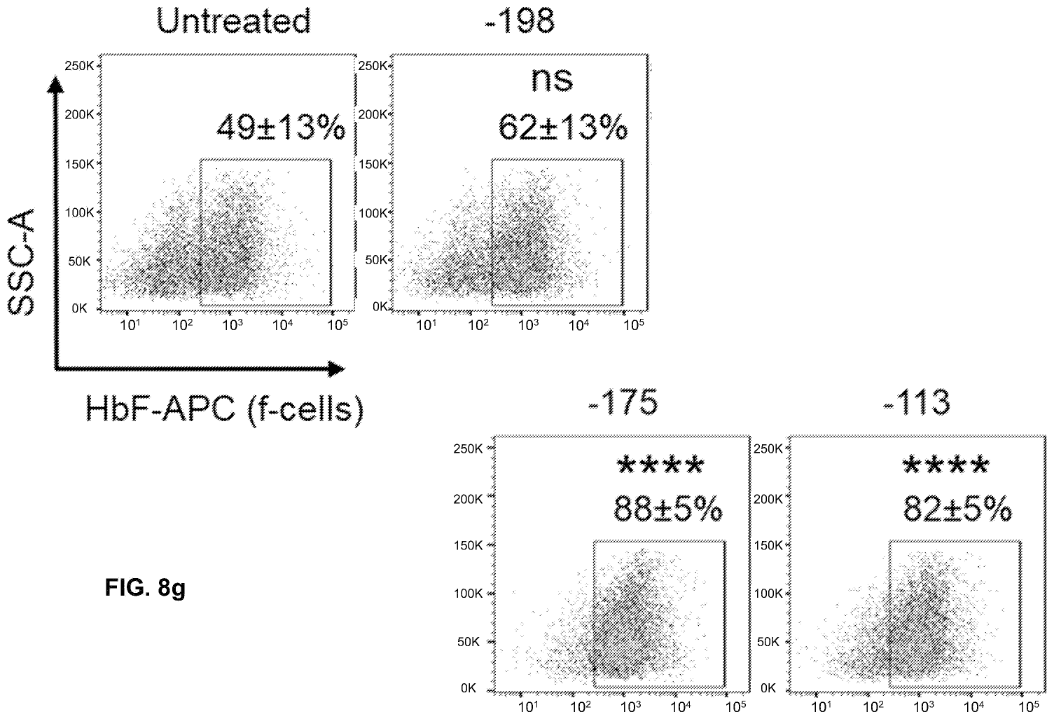

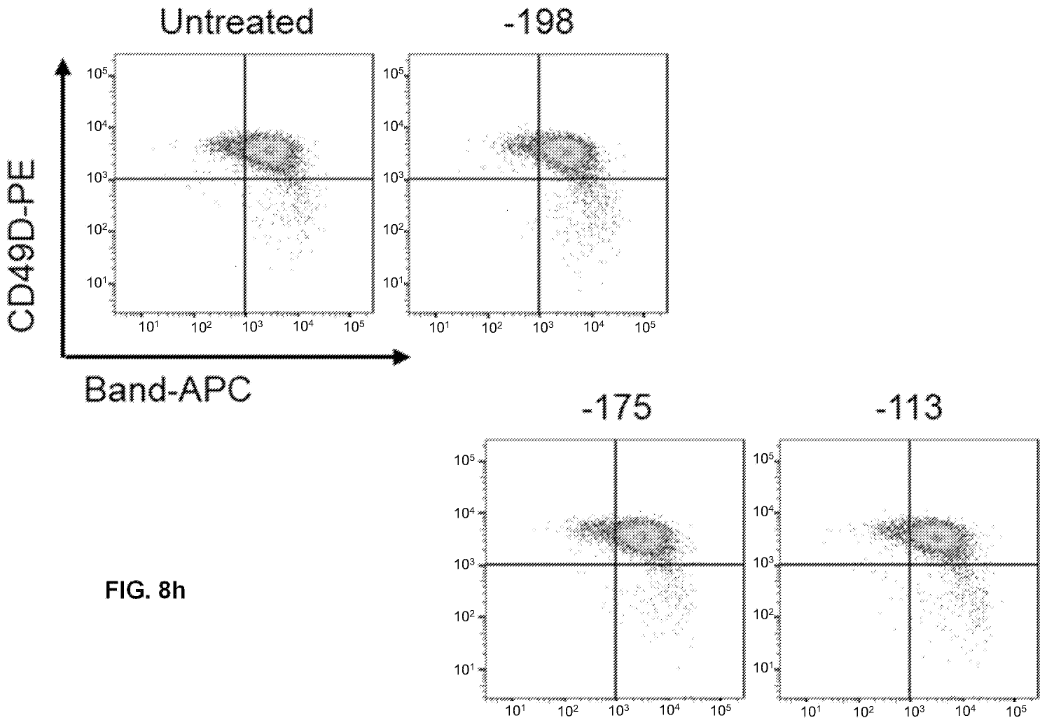

To test the base editing efficiency in CD34+ HSPCs from healthy donors, ribonucleic acid protein (RNP) complexes of base editor and sgRNA were electroporated separately targeting each of these 3 sites in the gamma-globin promoter. An editing efficiency of 9% was observed for the −198 site with ≤1% bystander mutations (FIG. 8a). For −175 site, the on-target editing was 30% with ≤1% bystander mutations (FIG. 8b). Using a guide targeting the −113 site, 5% editing was observed at the −112 nucleotide, 20% editing at the −113 nucleotide, and 17% editing at the −116 nucleotide (FIG. 8c). Further, <1% indels were measured following each of the three editing strategies (FIG. 8g). Each edited population was differentiated into mature erythroid cells over 21 days. Flow cytometric analysis of in vitro differentiated erythroid cells edited at the −198 site showed an increased population of F-cells from 49±13% in untreated controls to 62±13%. Editing at the −175 and −113 sites yielded increased F-cells, at 88±5% and 82±5%, respectively (FIG. 8d and FIG. 8h). Analytical IE-HPLC performed on the lysates of these cells revealed that the treatment targeting the −198 variant raised the HbF expression to 23±8%, the treatment targeting the −175 variant to 57±12%, and the treatment targeting the −113 variant to 39±6% compared to 14±5% in non-edited cells (FIG. 8e). Also, base editing did not alter the expression of erythroid maturation markers Band3 or CD49d (FIG. 8i). Overall, these results demonstrate that the greatest editing efficiency and HbF induction is obtained by the −175 TàC edit, followed by the −113 and −116 AàG edits, with the −198 TàC edit yield the least efficient editing and HbF induction.

Clonal Analysis of HPFH Mutants Erythroid Progeny Edited with ABE7.10.

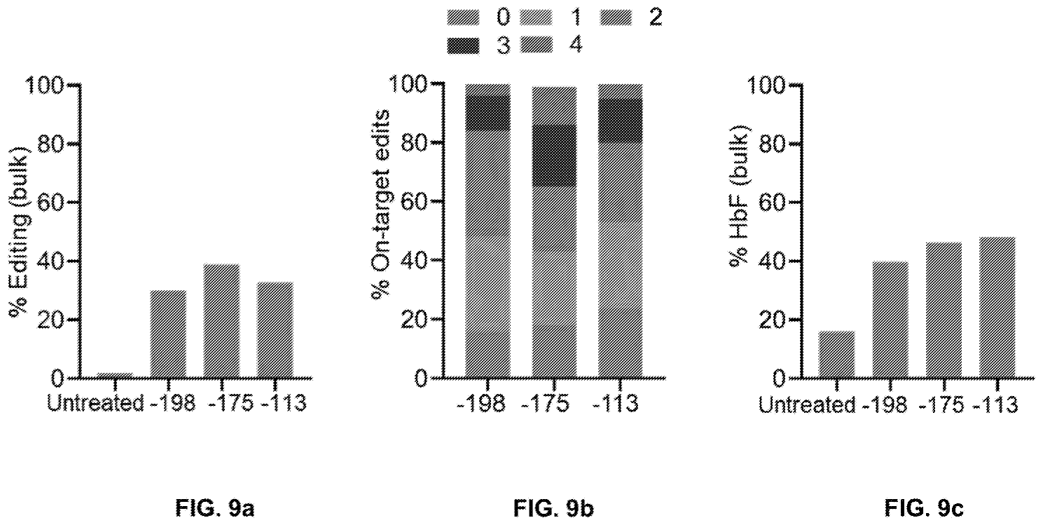

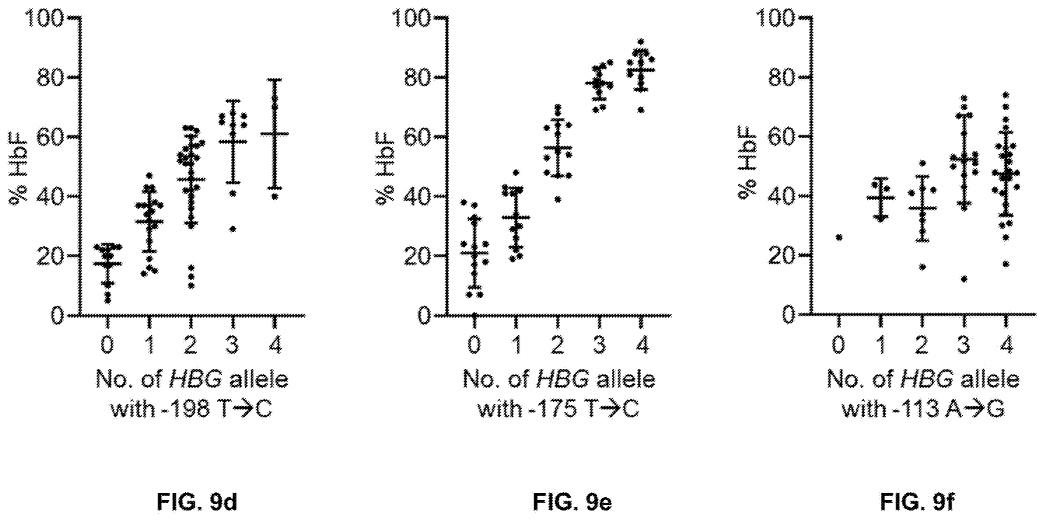

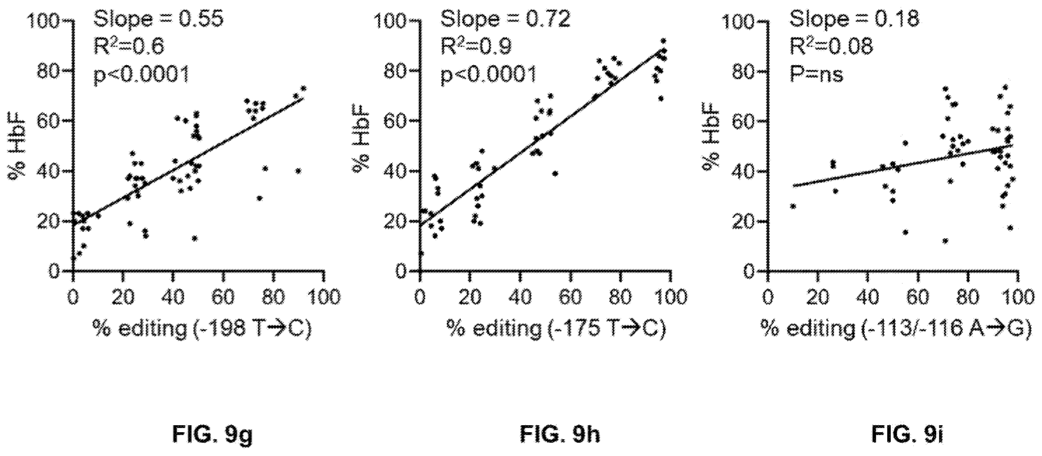

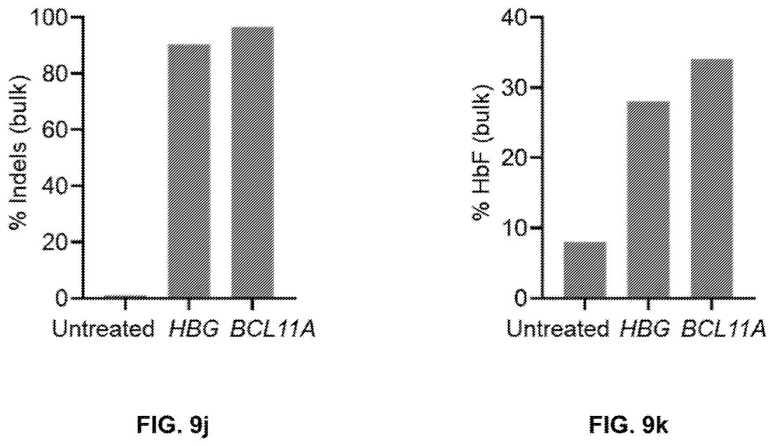

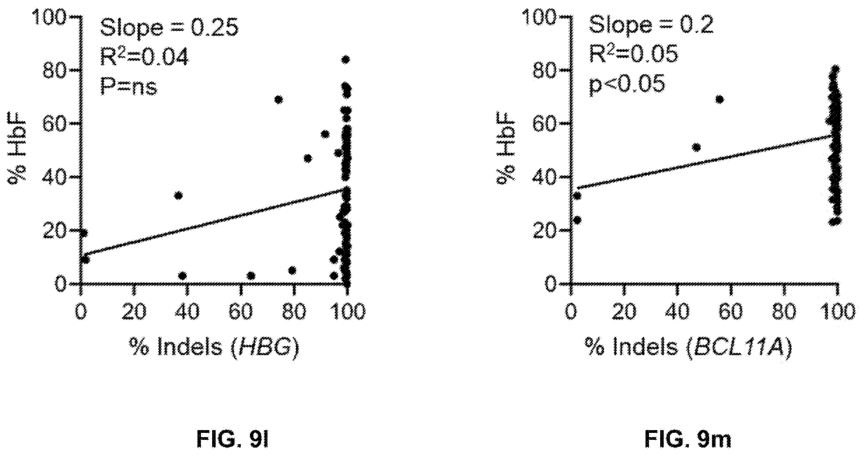

To investigate the editing frequency and HbF induction at clonal level, burst forming unit erythroid (BFUe) colony assay was performed in the CD34+ cells edited for all three HPFH variants. The overall on-target edits in bulk population were 30%, 39% and 33% for −198 T→C, −175 T→C and −113/−116 A→G respectively (FIG. 9a). The on-target editing frequency for all 3 sites at tandem HBG1/2 genes in individual BFUe clones was measured and observed edits in 4 copies of HBG genes resulting in 80% of cells edited with at least one copy of HBG gene (FIG. 9b). The % HbF induction in bulk population were 40% , 46% and 49% for −198 T→C. −175 T→C and −113/116 A→G respectively compared to 16% in untreated cells (FIG. 9c). Next, the % HbF in BFUe clones was measured and observed the dose dependent increase in HbF to the number of HBG gene edited for −198 and −175 variant (FIG. 9d&e). But, for −113/116 variant the HbF were not correlated with HbF induction and number of HBG genes edited indicating that the bystander mutations at position −112 (A9) at −113 site may perturb the −113 and or −116 variant mediated HbF induction (FIG. 9e). The HbF induced by −198 and −175 variant showed strong correlation with % base edits and −113/116 variants showed poor correlation due to mosaicism of on-target and bystander edits (FIG. 9g-i). For comparison, nuclease editing was performed using 3x NLS-SpCas9 nuclease to disrupt BCL11A binding site at HBG1/2 promoter and GAT motif at BCL11A enhancer. The indel frequency is >90% for both the locus (FIG. 9i) and induced HbF to 28% and 32% respectively (FIG. 9k) in bulk populations. Clonal analysis indicates that there is wide spectrum of HbF levels with >90% indels for both the edited sites (FIG. 9c&d) indicating that not all the indels contributes to HbF expression. These results suggest that installing a precise HPFH variant using base editor rather than creating random indels may be ideal for therapy. Although, all three HPFH variants induced HbF, but −175 T→C variant is more promising as the HbF induced >80% HbF with 4 copies of HBG edited compare to −198 T→C and −113/ −116 variants.

### ABE7.10 Generates −175 T→C HPFH Mutations in Normal Human Hematopoietic Stem Cells.

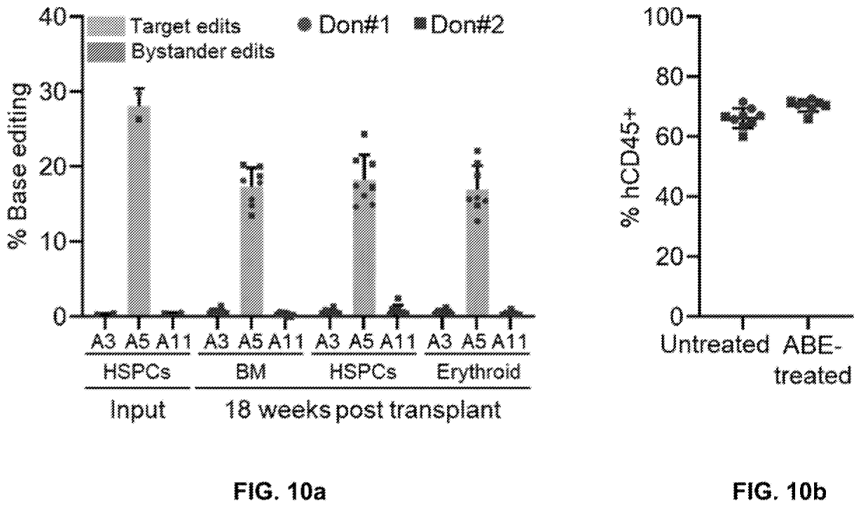

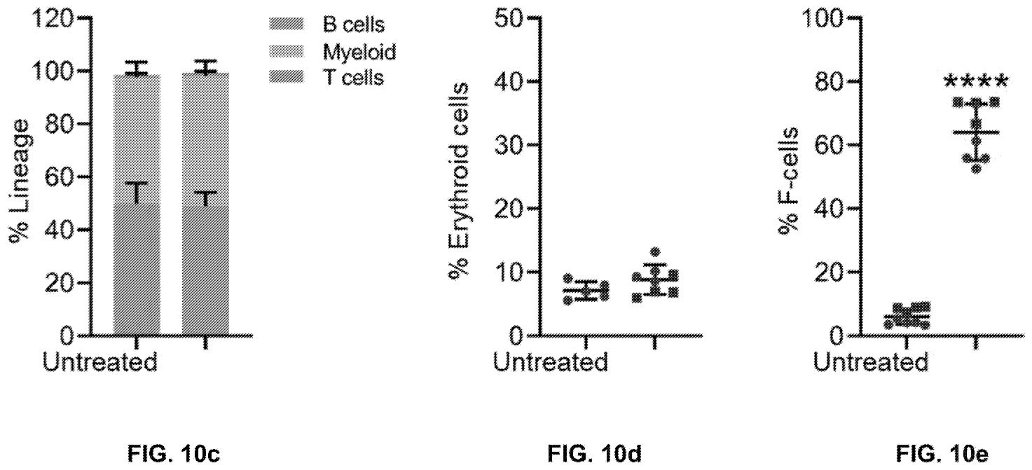

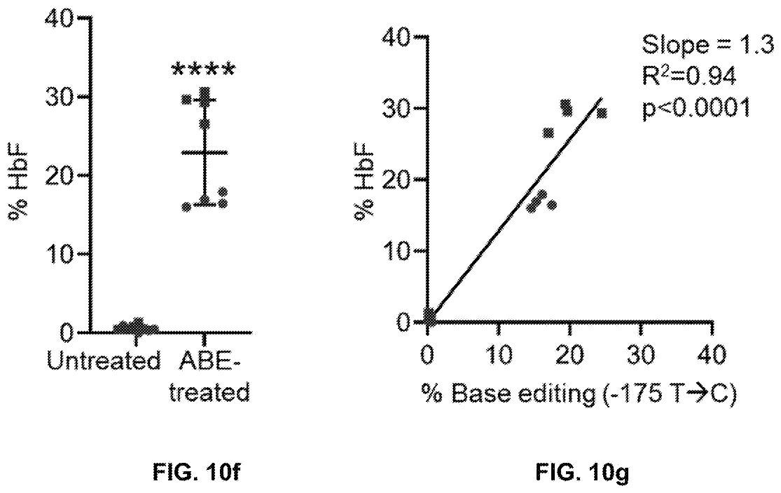

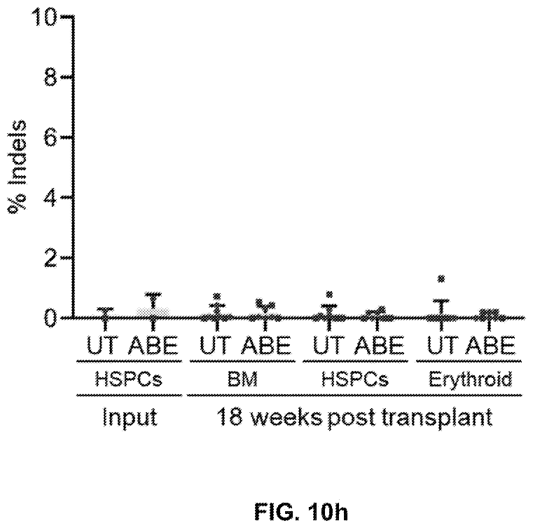

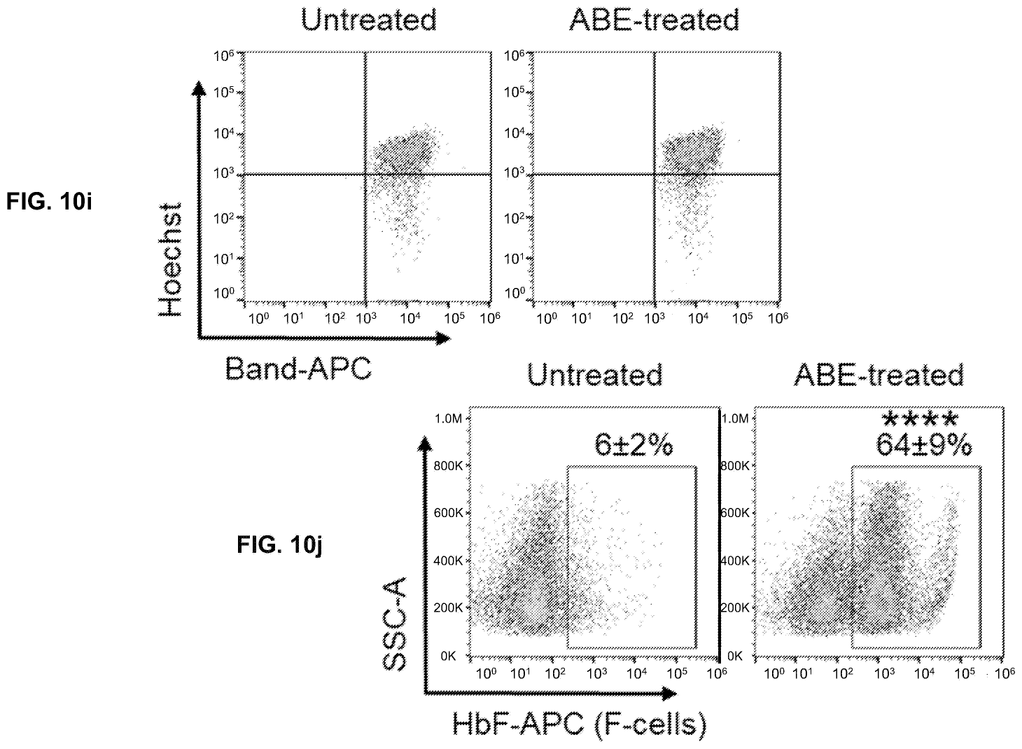

To assess the capacity to edit repopulating HSCs, −175 TàC edited CD34+ cells were transplanted into nonobese diabetic/severe combined immunodeficiency/I12rgamma−/−/KitW41/W41 (NBSGW) mice. The fraction of engrafted human cells and their editing frequencies was measured periodically in extracted blood. and after the mice were euthanized at 18 weeks. from bone marrow. The base editing frequencies at the −175 site of edited human donor in bone marrow declined from 28% after editing to 18% at 18 weeks after xenotransplantation (FIG. 10a). Editing frequencies were also similar in HSPCs and crythroid cells derived from them. suggesting that repopulating HSCs were edited at an allelic efficiency of 18% (FIG. 10a). The base editing spectrum in edited cells was similar before and after transplantation. with no clonal dominance in the latter populations. Indels frequencies were <1% and similar to that of cells before engraftment (FIG. 10h). No significant differences in human/mouse chimerism occurred between edited and nonedited donor derived CD45+ hematopoietic cell populations (FIG. 10b). Chimerism levels were similar for edited and nonedited donor T cells (hCD45+/CD3+), B cells (hCD45+/CD19+), myeloid cells (hCD45+/CD33+) (FIG. 10c), and erythroid cells (CD45−/CD235a+) in bone marrow at 18 weeks (FIG. 10d). Circulating human RBCs are short lived and difficult to detect in mouse xenotransplants. Therefore, immune-selected CD235a+ donor-derived erythroblasts were studied from the bone marrow of recipient mice at 18 weeks after transplantation. Mainly late stage hemoglobinized erythroblasts and reticulocytes were obtained, with no obvious differences in expression of maturation markers between the edited and untreated populations (FIG. 10i). The percentages of both HbF-immunostaining cells (F cells) and HbF protein compared with overall Hb were significantly increased in edited vs control erythroid cells generated in vivo (FIG. 10c&f, FIG. 10j). Considering that the HbF content was 23%±7% in erythroblasts with ˜18% base editing, it is likely that HbF was induced in all edited cells, thereby favoring therapeutic efficacy. The HbF content in erythroid cell lysates correlated with editing frequency based on measurements from individual mice (FIG. 10g). These xenotransplantation studies demonstrate that it is feasible to induce crythroid HbF expression by ABE7.10 RNP-mediated recreation of HPFH mutations to recruits TAL1 or GATA1 to the HBG1/HBG2 promoters, with no obvious alterations in hematopoietic development in vivo.

### ABE7.10 Generates −175 T→C′ HPFH Mutation in SCD Donor Derived Human Hematopoietic Stem Cells.

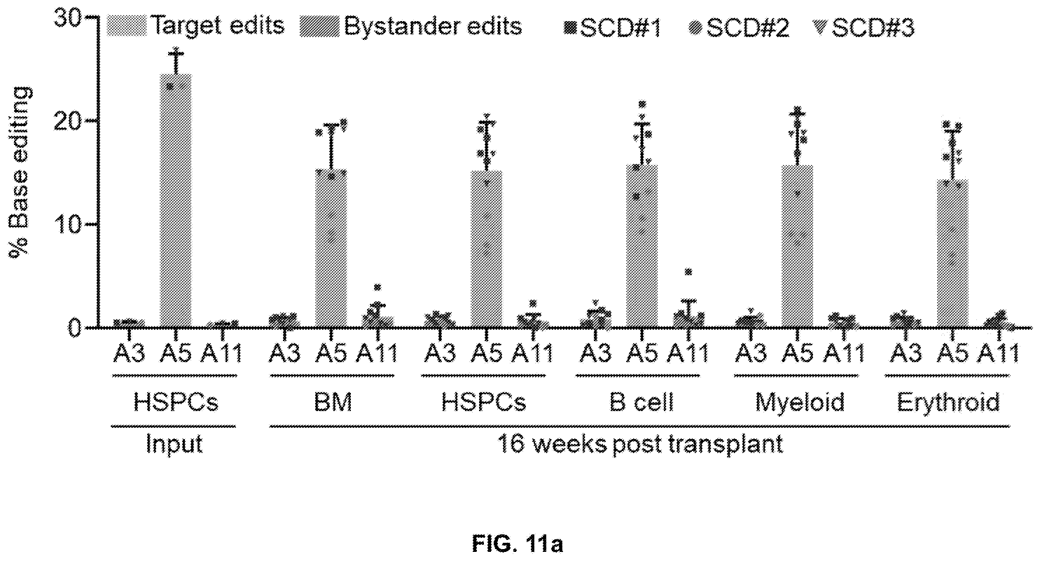

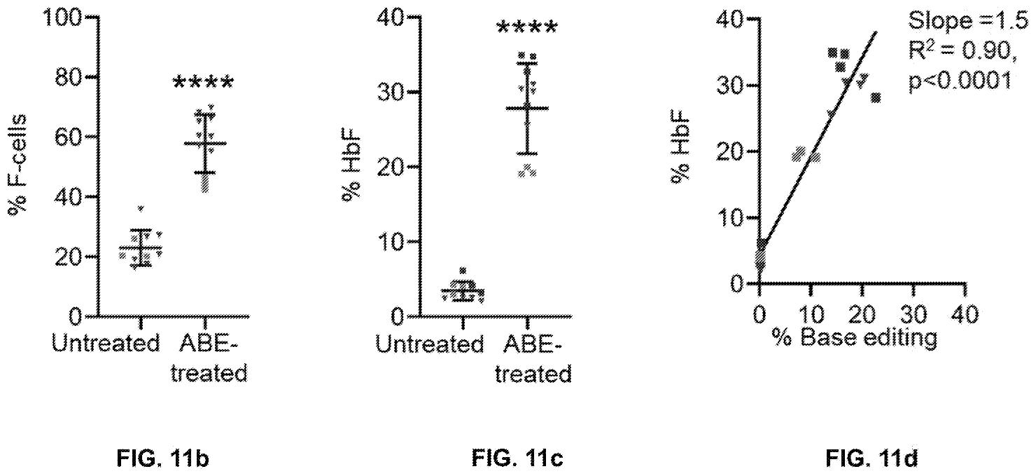

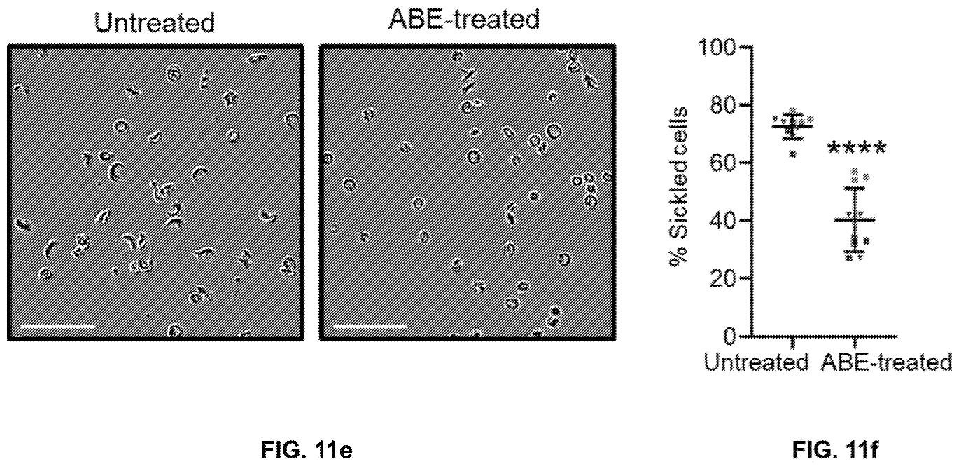

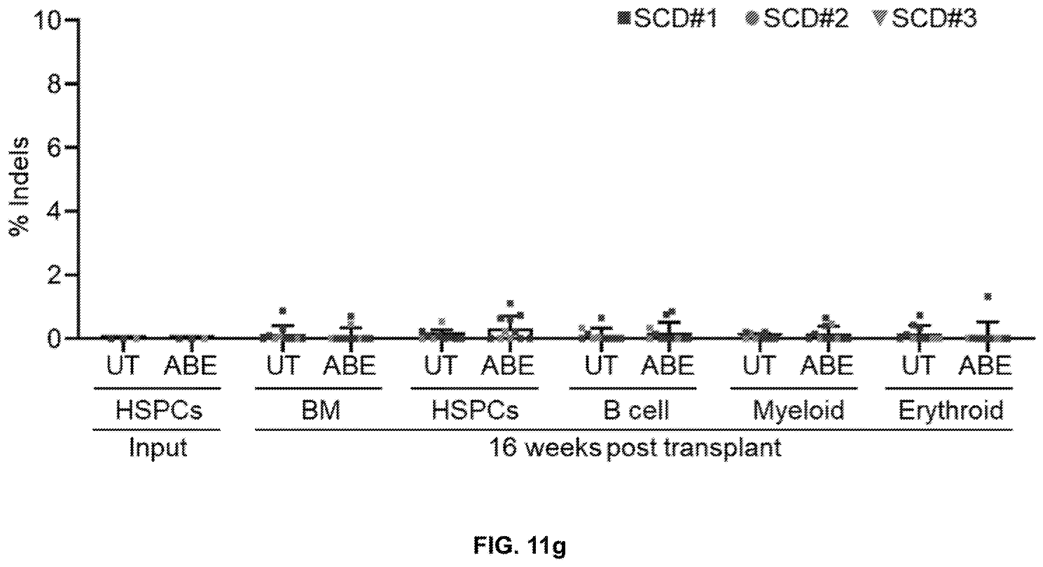

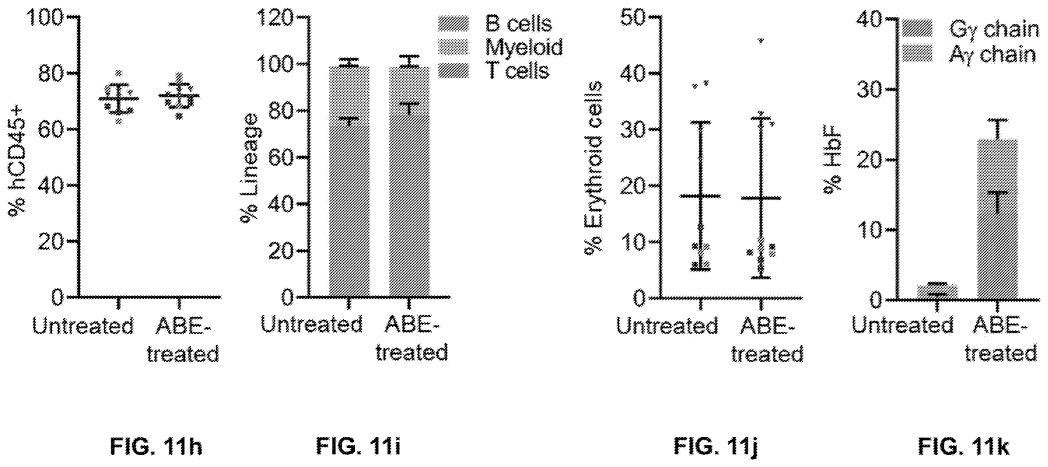

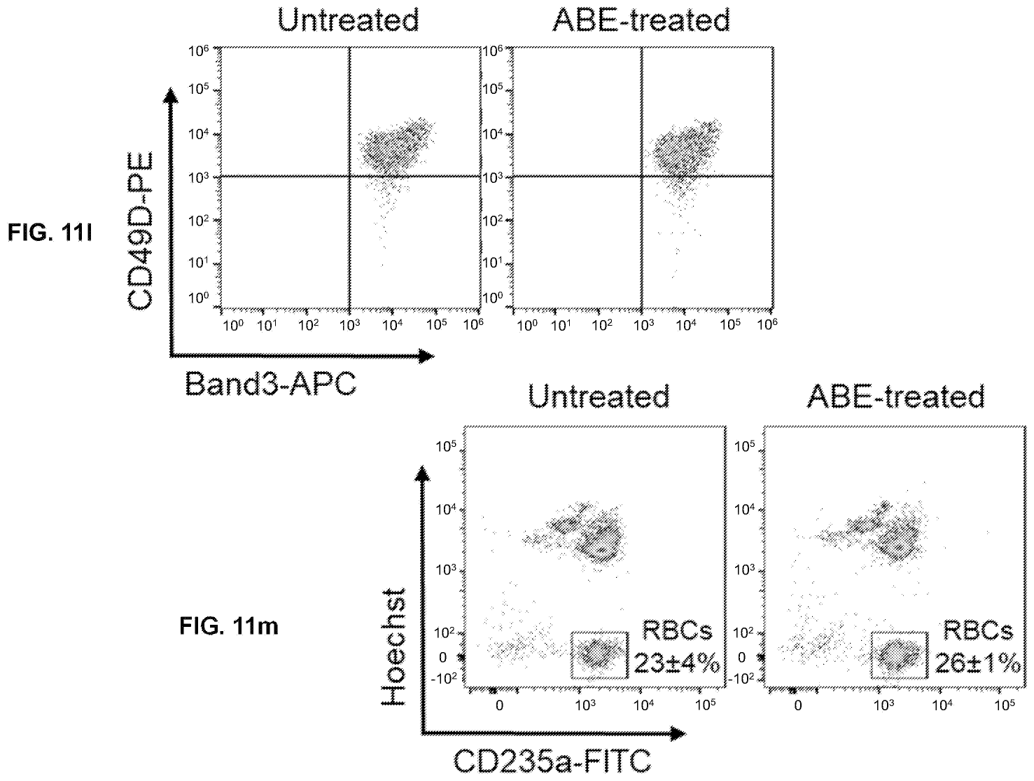

To test the anti-sickling properties of induced gamma-globin in a clinically relevant model. base editor RNPs targeting the −175 variant was electroporated into plerixafor-mobilized peripheral blood CD34+ HSPCs from individuals with SCD. The editing frequency before transplantation was 25% and 15% (60% of input cells) at 16+ weeks after transplantation (FIG. 11a). Editing frequencies were also similar in CD34+ donor cell-derived myeloid, crythroid, and B cells (FIG. 11a), indicating that editing did not alter the development of these lineages from HSCs. The spectrum of on-target base edits in SCD CD34+ HSPCs was similar to that observed after editing healthy CD34+ HSPCs and did not change appreciably after xenotransplantation (FIG. 11a). Indels frequencies were <1% and was similar to that of input (FIG. 11g). After xenotransplantation into NBSGW mice, edited and control CD34+ cells populated the bone marrow similarly and gave rise to similar fractions of human T, B, myeloid, and erythroid cells (FIG. 11h-j). Sixteen weeks after transplantation, bone marrow erythroblasts derived from gene-edited and control CD34+ HSPCs exhibited similar maturation profiles and enucleation as determined by flow cytometry (FIG. 11l&m). Erythroblasts derived from gene edited HSPCs CD34+ cells contained 58%±10% F cells as compared with 23%±6% in control nonedited erythroblasts (FIG. 11b). The HbF content was 28%±6% after gene editing vs 3%±1% in control erythrocytes (FIG. 11c). HbF induction in SCD erythroid cells correlated with base edit formation (FIG. 11d). similar to observations in erythroid cells derived from normal donor HSPCs (FIG. 10g). An in vitro sickling assay on erythroid cells purified from bone marrow indicates robust reduction of sickling in edited cells (40%±2%) compared to control cells (73%±4%) (FIG. 11e, f). Thus, HbF induction resulting from base editor RNP-induced recreation of the naturally occurring −175 variant at the HBG1 and HBG2 gene promoters inhibits erythroid cell sickling.

### Consequences of Single Strand DNA Break Induced by ABE7.10.

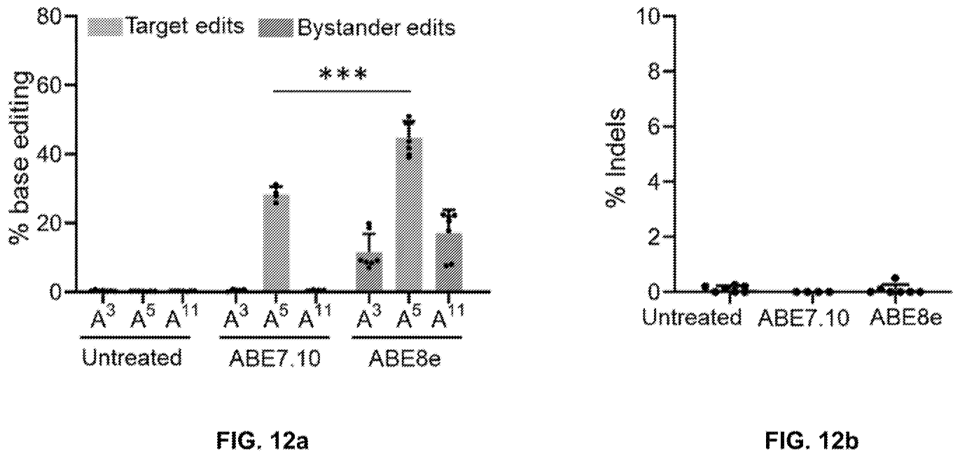

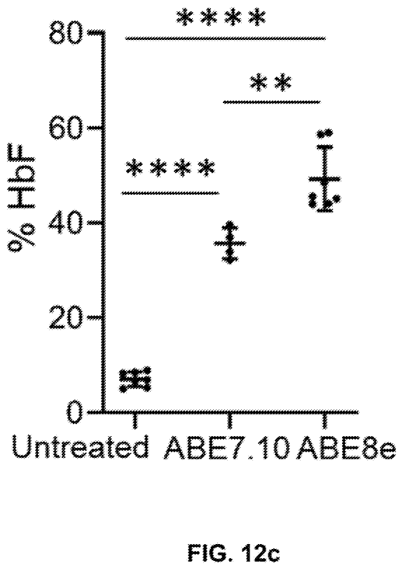

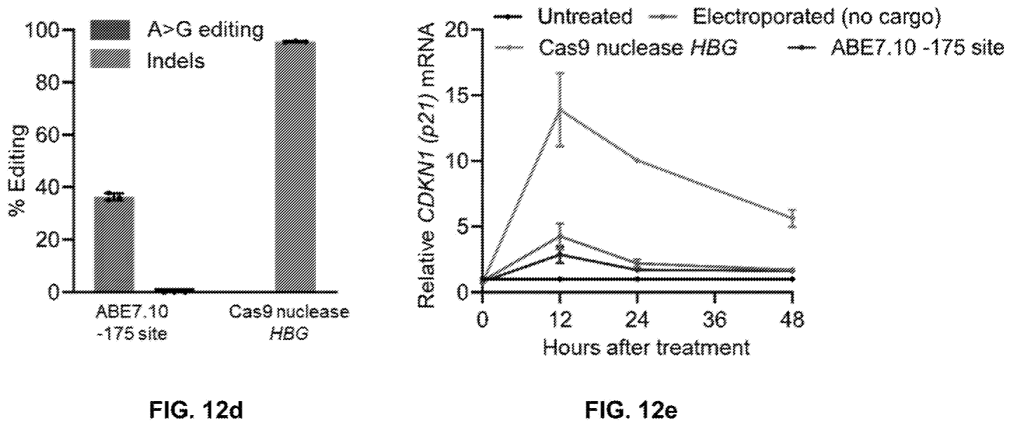

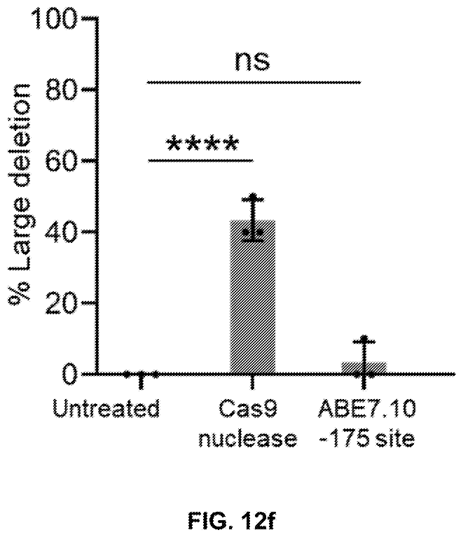

To determine the genotoxicity that may be induced by base editor nicking and/or deaminating the duplicated HBG gene, healthy donor CD34+ cells were electroporated with ABE7.10+sgRNA targeting −175 site and measured p21 mRNA levels using qPCR to determine the DNA damage response over time after base editing. In parallel, 3× NLS Cas9 nuclease+sgRNA targeting −115 site at HBG1/2 promoter were electroporated as a control. The on-target base editing frequency was 38% and indel frequency of Cas9 nuclease edited cells were 95% (FIG. 12d). Consistent with intact DNA damage response, transient induction of p21 transcript following Cas9 nuclease RNP electroporation targeting the HBG promoter in CD34+ HSPCs was observed, with peak levels 12 hours after electroporation (FIG. 12e). In contrast, the p21 induction after base editor RNP treatment did not exceed the level of induction caused by electroporation alone (FIG. 12e). Cas9 nuclease targeted to the tandem HBG1/2 genes and promoters can readily generate a 4.9 kb deletion of the intervening sequence. To determine whether base editor RNPs can also result in this deletion, qPCR was used to quantify the loss of the HBG2-HBG1 intergenic region approximately 366 bp upstream of the protospacers. While Cas9 nuclease targeting the −115 site generated the 4.9 kb deletion in 43%±6% of resulting alleles, ABE7.10 RNP targeted −175 site led to a deletion in 3%±6% of alleles (FIG. 12f). Thus, base editors lead to a much lower, but still measurable, 4.9 kb deletion relative to Cas9 nuclease.

Off-Target Base Editing Associated with ABE7.10 CD34+ Hematopoietic Stem and Progenitor Cells.

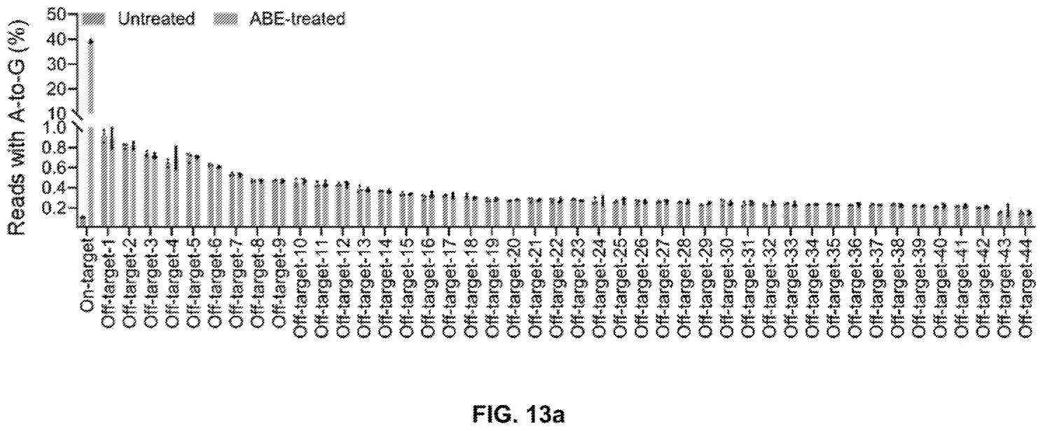

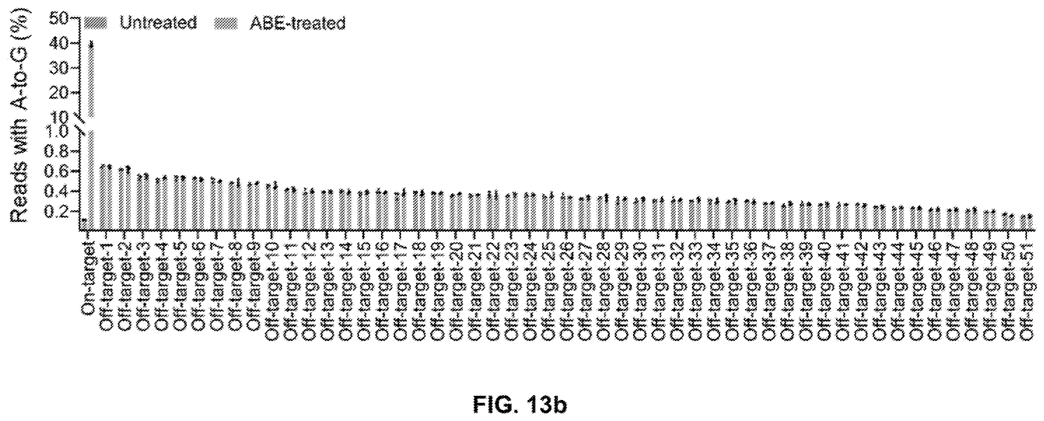

In silico analysis was performed using Cas9 off-finder and CIRCLE-seq to identify potential guide RNA-dependent off-target base editing sites and then directly evaluated each of these sites by targeted amplicon deep sequencing, 104 potential genomic off-target sites were identified with 3 or fewer mismatches relative to the on-target site and 96 off-targets by CIRCLE-seq. Amplicon deep sequencing was performed for each of these sites in genomic DNA samples isolated from cells with on-target editing (40%) and from negative control cells that were not edited. Evidence of off-target base editing in edited cells was not detected compared with negative control cells for all predicted off-targets. Based on sequencing reads sensitivity (0.2 to 0.9%), top 44 off-targets for in silico predicted and top 51 CIRCLE-seq predicted off-targets are shown in FIGS. 13a and 13b respectively.

### ABE8e Mediated Base Editing of HBG1/2 Promoter Improved the Editing Efficiency in CD34+ Cells and HbF Induction in Erythroid Progeny.

To improve the base editing efficiency in CD34+ HSPCs, a recently evolved adenosine base editor (ABE8e) was used, having higher editing kinetics compared to previous versions of ABEs. ABE8e+sgRNA targeting −175 site in CD34+ cells of healthy donor were electroporated. An on-target editing efficiency was observed up to 50% compared to 30% in ABE7.10 edited cells (FIG. 12a). The bystander edits at positions A3 and All occurring in 12% and 17% respectively in ABE8e edited cells compared to <1% in ABE7.10 edited cells (FIG. 12a). Low rates of indels were observed following editing ≤1% at both ABE8e and ABE7.10 edited cells (FIG. 12b). Analytical IE-HPLC performed on the lysates of mature erythroid cells revealed that the treatment targeting with ABE8e raised the HbF expression to 49±7%, the treatment targeting with ABE7.10 induced to 36±3% compared to 7±2% in untreated cells (FIG. 12c). Although, by stander edits were found at higher rate when edited with ABE8e but doesn't compromise with HbF induction. Together, these results demonstrate that the ABE8e improved the on-target editing frequency and HbF induction in erythroid progeny.

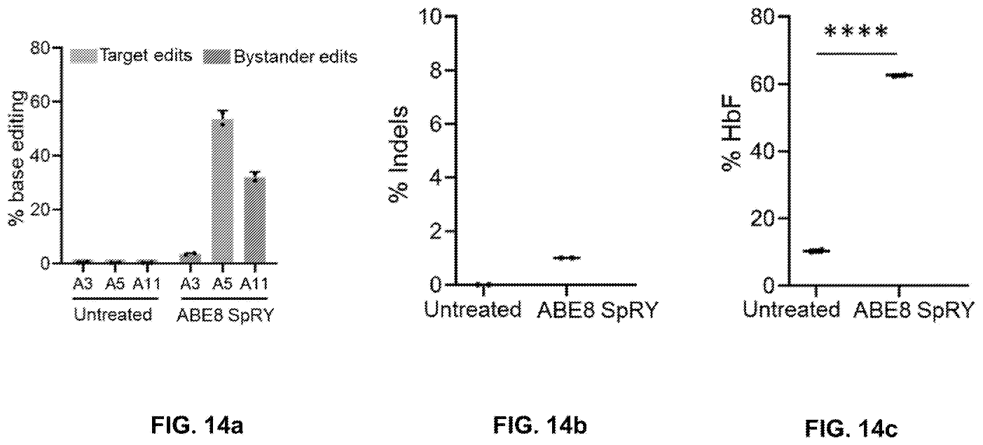

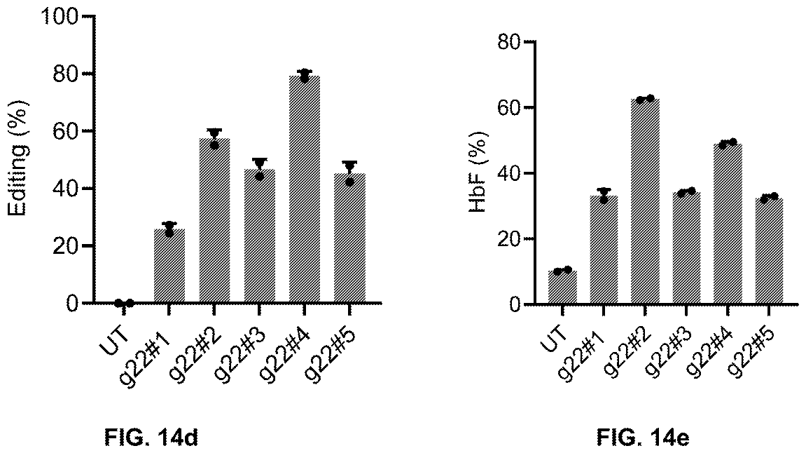

Further, FIG. 14 (a-c) shows ABE8 PAM-less SpCas9 (ABE8 SpRY) mediated based editing of HBG1/2 promoter in CD34+ cells and HbF induction in erythroid progeny. See also Tables 4 and 5. FIG. 14d and FIG. 14e show base editing using SpRY mRNA with different guides targeting for −175 site (see Table 6; base underlined is on target nucleotide ( −175 T−C); bases in bold, larger font are potential by stander nucleotides). Non-patent literature to Walton et al. entitled “Unconstrained genome targeting with near-PAMless engineered CRISPR-Cas9 variants”, Science, 2020, v. 368 (no. 6488), pp. 290-296 is hereby incorporated by reference herein for all purposes.

TABLE 6
Guides used for SpRY mRNA base editing.

| Guide | Sequence | SEQ ID NO: |
|---|---|---|
| g22#1 (A) | AGATATTTGCATTGAGATAG | SEQ ID NO: 13 |
| g22#2 (C) | GATATTTGCATTGAGATAGT | SEQ ID NO: 14 |
| g22#3 (D) | CAGATATTTGCATTGAGATA | SEQ ID NO: 15 |
| g22#4 (E) | ACAGATATTTGCATTGAGAT | SEQ ID NO: 16 |
| G22#5 (F) | GACAGATATTTGCATTGAGA | SEQ ID NO: 17 |

### Discussion

Developing effective approaches to induce HbF to treat beta-hemoglobinopathies has been a “holy grail” in the field for more than 50 years. A major breakthrough in the field came from genome-wide association studies (GWAS) showing that polymorphisms in the extended beta-like globin gene cluster, BCL11A, and the HBS1L-MYB intergenic region are associated with increased erythroid HbF levels. Here, ABEs were used to generate naturally occurring mutations in the HBG promoter leading to HbF production.

First, the proof of concept was recapitulated to recreate the HPFH mutations in HBG promoter to induce HbF in adult erythroid progenitor cell line. Precise point mutations with extremely low frequencies of resulting indels were achieved using base editor electroporation. Although the editing efficiency was different for each guide all three HPFH mutations induced significant levels of HbF. Very low rate of bystander mutation was observed within the editing window for sgRNA −198 and −175, but the −113 guide yielded significant bystander edits with one at position 5 in the protospacer falls in the BCL11A binding motif (TGACC) appearing to increase HbF production even more than the natural variant. Clonal analysis in hemizygous HUDEP-2 cells displayed significant HbF production for all three HPFH mutations. Interestingly, the bystander mutation at position 9 in the −113 protospacer appeared to decrease HbF levels relative to alleles edited only at the positions 5 and 8. This effect is likely due to disruption of the new GATA1 binding motif introduced by the −113 polymorphism, though it is possible that some other transcription factor binding is modulated. Notably, −175 T→C mutation yielded the most robust HbF induction compared to other 2 mutations. Also, the present disclosure gains insight to the regulation of gamma-globin gene by −175 Tà C variant that recruits TAL1. The GATA motif proximal to TAL1 binding site was found to be essential for gamma-globin expression through recruitment of NFYA.

Second, precise HPFH point mutations in human primary HSPCs were created using base editors. Editing frequencies at the −175 site were higher (up to 40%) compared to the other 2 sites. HbF induction is correlated with the editing rates in erythroid progeny for all 3 sites and potent HbF induction was observed with the −175 TàC HPFH mutation in both primary CD34+-derived erythroid cells and HUDEP-2 cells. In vivo studies in NBSGW mice model revealed that editing frequency was retained up to 20% in long term repopulating HSCs and induced up to 22% HbF content in erythroid progeny. Base editing of HSPCs didn't skew the erythroid maturation or enucleation of terminal differentiated erythroid cells.

Third, by using gRNAs targeting the −175 TàC mutation, a robust, pancellular HbF reactivation and a concomitant reduction in beta^{S}-globin levels were achieved, recapitulating the phenotype of asymptomatic SCD patients co-inherited with HPFH mutations. In vivo experiments demonstrated that editing frequency was maintained up to 20% 16 weeks post transplantation and induced HbF at a therapeutic level (>30%) without affecting erythroid maturation. Additionally, the DNA lesions generated by base editors do not appear to evoke a p53 induced DNA damage response as Cas9 does. The 4.9 kb deletion intervening HBG2-HBG1 locus in −175 sgRNA treated cells is much rarer than the deletions generated by Cas9 nuclease. Therapeutic levels of fetal hemoglobin were observed in SCD erythroid progeny with the −175 TàC edit, which was the most attractive of three sites assessed. The recent version of ABE (ABE8e) significantly improved the editing rate with concomitant induction of HbF compared to previous version of ABEs. An autologous transplantation following this edit in mobilized patient HSPCs serves as an attractive new therapeutic. This strategy may be used to treat both beta-thalassemia in addition to SCD, as elevated HbF induction compensates for the deficient beta-globin chains.

### Conclusion

Overall, the present disclosure demonstrates the potent expression of HbF achieved based on the −175 TàC HPFH mutation and identify the underlying mechanism based on altered transcription factor binding. In exemplary embodiments, this edit is the basis for an autologous stem cell therapeutic for beta-hemoglobinopathies.

All publications, patents, patent applications, and other references cited in this application are incorporated herein by reference in their entirety for all purposes to the same extent as if each individual publication, patent, patent application or other reference was specifically and individually indicated to be incorporated by reference in its entirety for all purposes. Citation of a reference herein shall not be construed as an admission that such is prior art to the present disclosure.

It should be appreciated that all examples in the present disclosure are provided as non-limiting examples. Those skilled in the art will recognize or be able to ascertain using no more than routine experimentation many equivalents to the specific embodiments of the invention described herein. Such equivalents are intended to be encompassed by the following claims.

1. A cell derived from a subject having a hemoglobinopathy, the cell comprising at least one synthetic allele of an HBG1 or HBG2 gene, said synthetic allele comprising a T→C mutation at a position selected from:
−175 base pairs of the promoter region of the HBG1 or HBG2 gene;
HBG2: GRCh/hg19; chr11:5,276, 186; and
HBG1: GRCh/hg19; chr11:5,271,262.

2. The cell of claim 1, wherein the T→C mutation is at position 175 bp (−175) upstream from 5′ UTR region or 228 bp (−228) upstream from transcription start site of HBG1/2 gene relative to the proximal promoter sequence
(SEQ ID NO: 12)
5′-CTATCTCAATGCAAATATCT-3′.

3. The cell of claim 1, wherein the synthetic allele further comprises a T→C mutation at position 12 of the proximal promoter sequence.

4. The cell of claim 1, wherein the cell is selected from the group consisting of a CD34+ hematopoietic stem and progenitor cell (CD34+ HSPC) and a cell in the erythroid lineage.

5. The cell of claim 1, wherein the cell is derived from the group consisting of bone marrow, peripheral blood, mobilized peripheral blood, cord blood, and induced pluripotent stem cells (iPSCs).

6. The cell of claim 1, characterized in that a differentiated cell in the erythrocyte lineage derived therefrom expresses a quantity of gamma-globin protein greater than the quantity of gamma-globin protein expressed by a cell in the erythrocyte lineage derived from the same subject which lacks the at least one synthetic allele.

7. A composition of cells, wherein at least 20%, 25%, 30%, 35%, 40%, 45%, 50%, 55%, 60%, 65%, 70%, 75%, 80%, 85%, 90%, 95%, or more alleles of HBG1 and HBG2 genes within said composition of cells comprise a T→C mutation at −175 base pairs of the promoter region of the HBG1 or HBG2 gene.

8. The composition of cells of claim 7, wherein the cells are derived from a subject that does not carry said T→C mutation.

9. The composition of cells of claim 7, wherein each cell within said composition of cells has at least one modified allele.

10. The composition of cells of claim 7, having an average of 1.0 to 2.0 modified alleles per cell.

11. The composition of cells of claim 7, wherein the cells are selected from the group consisting of a CD34+ hematopoietic stem and progenitor cells (CD34+ HSPCs) and cells in the erythroid lineage.

12. The composition of cells of claim 7, wherein the cells are derived from the group consisting of bone marrow, peripheral blood, mobilized peripheral blood, cord blood, and induced pluripotent stem cells (iPSCs).

13. The composition of cells of claim 7, wherein at least 20%, 25%, 30%, 35%, 40%, 45%, 50%, 55%, 60%, 65%, 70%, 75%, 80%, 85%, 90%, or 95%, of cells in the composition express a gamma-globin protein when differentiated into an erythrocyte lineage.

14. The composition of claim 7, wherein at least 20%, 25%, 30%, 35%, 40%, 45%, 50%, 55%, 60%, 65%, 70%, 75%, 80%, 85%, 90%, or 95% of hemoglobin produced by the cells comprises a gamma-globin protein.

15. A composition, comprising: a population of cells characterized by a T→C mutation at −175 base pairs of the promoter region of the HBG1 or HBG2 gene, said T→C mutation occurring at a rate of at least 40%, 50%, or 60% in the bulk population of cells.

16. The composition according to claim 15, wherein the cells are derived from a subject that does not carry said T→C mutation.

17. The composition according to claim 15, wherein the population of cells consists essentially of CD34+ cells.

18. The composition of claim 15, further comprising a pharmaceutically acceptable carrier.

19. The composition according to claim 15, wherein the rate is measured by next generation sequencing of bulk genomic DNA from a sample of the composition.

20-60. (canceled)
